(19) United States
(12) Patent Application Publication
(10) Pub. No.: US 2023/0109152 A1
(43) Pub. Date: Apr. 6, 2023

(54) ULTRASONIC GENETICALLY ENCODED CALCIUM INDICATORS

(71) Applicant: California Institute of Technology, Pasadena, CA (US)

(72) Inventors: Zhiyang Jin, Pasadena, CA (US); Mikhail G. Shapiro, Pasadena, CA (US); Anupama Lakshmanan, Seattle, WA (US); Teresa Tran, Los Angeles, CA (US)

(21) Appl. No.: 17/937,975

(22) Filed: Oct. 4, 2022

Related U.S. Application Data
(60) Provisional application No. 63/252,499, filed on Oct. 5, 2021.

(51) Int. Cl. C12Q 1/6897; C12N 5/071; C12N 15/62; C12N 15/85; A61B 8/08; C07K 14/005; C07K 14/215; C07K 14/195

(52) U.S. Cl. CPC C12Q 1/6897; C12N 5/0686; C12N 15/625; C12N 15/85; A61B 8/08; C07K 14/005; C07K 14/215; C07K 14/195; C12N 2800/107; C12N 2503/00; C07K 2319/00

(57) ABSTRACT

Disclosed herein include methods, compositions, and kits suitable for use in calcium imaging. There are provided, in some embodiments, Ca²⁺-sensing GvpC proteins. Disclosed herein include Ca²⁺-sensing gas vesicles (GVs) comprising Ca²⁺-sensing GvpC proteins. In some embodiments, the Ca²⁺-sensing GvpC protein is capable of undergoing a first allosteric conformational change upon the Ca²⁺-binding domain binding Ca²⁺ that causes the Ca²⁺-sensing GV to change from a GV stiff state to a GV soft state. One or more of the mechanical, acoustic, surface, and magnetic properties of a Ca²⁺-sensing GV can differ between the GV soft state and the GV stiff state.

### RELATED APPLICATIONS

This application claims the benefit under 35 U. S. C. § 119(e) of U.S. Provisional Patent Application Ser. No. 63/252,499, filed Oct. 5, 2021, the content of this related application is incorporated herein by reference in its entirety for all purposes.

### STATEMENT REGARDING FEDERALLY SPONSORED R&D

This invention was made with government support under Grant No(s). EB018975 & NS120828 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-302457-US, created Oct. 4, 2022, which is 84.0 kilobytes in size. The information in the electronic format of the Sequence Listing is incorporated herein by reference in its entirety.

### BACKGROUND

### Field

The present disclosure relates generally to the field of calcium imaging.

### Description of the Related Art

Calcium ions (Ca²⁺) play an important role in almost every aspect of cellular signaling and the dynamics of calcium can be used as a functional readout for many biological processes. In non-excitable cells, cytosol calcium concentration is widely connected with cell functions through numerous signaling pathways, such as T-cell activation and insulin secretion from beta cells. In excitable cells, calcium does not only regulate vital functions like synaptic plasticity, but also is tightly coupled with electrical signals that maps out the neural activity across the brain. Imaging technologies enabling whole-organism observation of calcium dynamics in specific cells represents a “holy grail” of biotechnology development. Currently, fluorescent genetically encoded calcium indicators (GECIs) play a dominant role in functional imaging by connecting optical signals to the biological processes inside specific, genetically defined cells. When combined with the latest optical techniques, GECIs enable single-cell Ca²⁺ imaging typically in volumes smaller than 1 mm³, at depths shallower than 1 mm. Alternatively, GECIs combined with implanted fiber photometry report the aggregate activity of genetically defined cellular populations in deep body regions with dimensions on the order of 200 μm. While both of these approaches have enabled major biological discoveries, they fall short of providing simultaneous access to large volume in deep tissue due to the physical limitations of light scattering. There is a need for compositions, methods, and systems for calcium imaging in deeper and/or larger regions (e.g., across the entire brain).

In addition to fluorescence imaging with GECIs, there are ongoing research on calcium imaging with other imaging modalities including magnetic resonance imaging (MRI) and photoacoustic tomography (PAT). MM provides fully non-invasive, whole-organism access, but at this stage the calcium indicators for MRI require fairly high concentrations, are difficult to encode genetically and involve acute intracranial injections. The sensitivity of MRI to motion also means that most scans are performed on anesthetized animals, which might affect the results in some cases, such as in the brain when imaging calcium-coupled neural activities. PAT combines optical excitation with acoustic detection to enable the imaging of optical chromophores in tissues with the deep-tissue resolution of ultrasound and a potential advantage of PAT is its ability to image existing GECI-like proteins. However, this requires overcoming the strong background of hemoglobin by greatly shifting the GECI wavelengths, which is in progress. Both of those imaging modalities require sophisticated equipment and many other equipment used in the experiments have to be specially designed for compatibility, which makes it more challenging to apply those techniques in a broader range of applications. There is a need for compositions, methods, and systems for calcium imaging which do not require sophisticated equipment, do not require high concentrations of calcium indicators to be administered, do not require subjects to be anesthetized, are not difficult to encode genetically, do not involve acute intracranial injections, and do not suffer from the strong background of hemoglobin-based signals.

Type 1 diabetes (T1D) is an autoimmune disease that affects one in 6000 people worldwide, and beta cell replacement strategies have been a major direction for the development of T1D therapeutics. While islet transplantation is an effective approach for beta cell replacement, the long-term therapeutic benefit remains challenging and the door shortage imposes a bottleneck for widespread adoption. Thus, transplants based on encapsulated engineered cells, such as stem cell (SC)-derived beta cells and beta-mimetic designer cells, have been intensively investigated as an alternative for beta cell replacement. However, those cells are usually dispersed throughout the peritoneum upon transplantation, making noninvasive and long-term graft monitoring extremely challenging. There is a need for compositions, methods, and systems for noninvasive monitoring of cell therapies.

### SUMMARY

There are provided, in some embodiments, nucleic acid compositions. The nucleic acid composition can comprise: one or more promoters operably connected to one or more gas vesicle (GV) polynucleotides comprising: a Ca²⁺-sensing GvpC gene encoding a Ca²⁺-sensing GvpC protein. In some embodiments, the Ca²⁺-sensing GvpC protein is capable of associating with a gas vesicle structural (GVS) protein to form a Ca²⁺-sensing gas vesicle (GV).

There are provided, in some embodiments, nucleic acid compositions. The nucleic acid composition can comprise: one or more promoters operably connected to one or more gas vesicle (GV) polynucleotides comprising: one or more gas vesicle assembly (GVA) gene(s) encoding one or more GVA protein(s), one or more gas vesicle structural (GVS) gene(s) encoding one or more GVS protein(s) selected from GvpA and GvpB, and/or a Ca²⁺-sensing GvpC gene encoding a Ca²⁺-sensing GvpC protein. In some embodiments, the one or more GVA protein(s), the one or more GVS protein(s), and the Ca²⁺-sensing GvpC protein are capable of forming Ca²⁺-sensing gas vesicles (GVs) upon expression in a cell or a cell-like environment.

Disclosed herein include compositions. In some embodiments, the composition comprises: a Ca²⁺-sensing GvpC protein, wherein the Ca²⁺-sensing GvpC protein is capable of associating with a gas vesicle structural (GVS) protein to form a Ca²⁺-sensing gas vesicle (GV).

Disclosed herein include compositions. In some embodiments, the composition comprises: a Ca²⁺-sensing gas vesicle (GV) comprising a Ca²⁺-sensing GvpC protein and one or more GVS protein(s) selected from GvpA and GvpB.

In some embodiments, the Ca²⁺-sensing GV comprises a gas enclosed by a protein shell comprising the Ca²⁺-sensing GvpC protein and a GVS protein selected from GvpA and GvpB. In some embodiments, the Ca²⁺-sensing GvpC protein comprises a Ca²⁺-binding domain. In some embodiments, the Ca²⁺-binding domain is capable of binding 1, 2, 3, 4, 5, or 6 Ca²⁺ ions. In some embodiments, the Ca²⁺-sensing GvpC protein is capable of undergoing a first allosteric conformational change upon the Ca²⁺-binding domain binding Ca²⁺ that causes the Ca²⁺-sensing GV to change from a GV stiff state to a GV soft state. In some embodiments, the Ca²⁺-binding domain binding Ca²⁺ comprises the Ca²⁺-binding domain binding 1, 2, 3, 4, 5, or 6 Ca²⁺ ions. In some embodiments, the Ca²⁺-sensing GvpC protein is capable of undergoing a second allosteric conformational change upon the Ca²⁺-binding domain releasing bound Ca²⁺ that causes the Ca²⁺-sensing GV to change from a GV soft state to a GV stiff state. In some embodiments, the Ca²⁺-binding domain releasing bound Ca²⁺ comprises the Ca²⁺-binding domain releasing 1, 2, 3, 4, 5, or 6 bound Ca²⁺ ions. In some embodiments, one or more of the mechanical, acoustic, surface, and magnetic properties of the Ca²⁺-sensing GV differ between the GV soft state to the GV stiff state. In some embodiments, the acoustic contrast of the Ca²⁺-sensing GV is capable of reversibly changing in response to local Ca²⁺ concentrations. In some embodiments, a Ca²⁺-sensing GV is capable of: (i) binding Ca²⁺, optionally 1, 2, 3, 4, 5, or 6 Ca²⁺ ions; and (ii) releasing bound Ca²⁺, optionally 1, 2, 3, 4, 5, or 6 bound Ca²⁺ ions, recurrently in response to changes in Ca²⁺ dynamics.

In some embodiments, the Ca²⁺-sensing GvpC protein comprises an interaction domain configured to: bind the Ca²⁺-binding domain upon the Ca²⁺-binding domain binding Ca²⁺, optionally upon the Ca²⁺-binding domain binding 1, 2, 3, 4, 5, or 6 Ca²⁺ ions, optionally the first allosteric conformational change comprises the interaction domain binding the Ca²⁺-binding domain; and detach from the Ca²⁺-binding domain upon the Ca²⁺-binding domain releasing bound Ca²⁺, optionally upon the Ca²⁺-binding domain releasing 1, 2, 3, 4, 5, or 6 bound Ca²⁺ ions, optionally the second allosteric conformational change comprises the interaction domain detaching from the Ca²⁺-binding domain.

In some embodiments, the Ca²⁺-sensing GvpC protein comprises an interaction domain configured to: detach from the Ca²⁺-binding domain upon the Ca²⁺-binding domain binding Ca²⁺, optionally upon the Ca²⁺-binding domain binding 1, 2, 3, 4, 5, or 6 Ca²⁺ ions, optionally the second allosteric conformational change comprises the interaction domain detaching from the Ca²⁺-binding domain; and bind the Ca²⁺-binding domain upon the Ca²⁺-binding domain releasing bound Ca²⁺, optionally upon the Ca²⁺-binding domain releasing 1, 2, 3, 4, 5, or 6 bound Ca²⁺ ions, optionally the first allosteric conformational change comprises the interaction domain binding the Ca²⁺-binding domain.

In some embodiments, the Ca²⁺-sensing GvpC protein comprises: an N-terminal domain; a C-terminal domain; and a central domain situated between N-terminal domain and the C-terminal domain, wherein the central domain comprises two or more tandem repeat regions. In some embodiments, each repeat region is an alpha-helical polypeptide. In some embodiments, the two or more tandem repeat regions: are 2, 3, 4, 5, or 6, tandem repeat regions; and/or share an amino acid sequence identity of at least about 50%. In some embodiments, the Ca²⁺-binding domain is N-terminal of the N-terminal domain. In some embodiments, the Ca²⁺-binding domain is C-terminal of the C-terminal domain. In some embodiments, the Ca²⁺-binding domain is connected to the N-terminal domain or the C-terminal domain via a first linker. In some embodiments, the interaction domain is situated within the central domain. In some embodiments, the interaction domain is situated within one of the two or more repeat regions. In some embodiments, the interaction domain is flanked by a second linker and a third linker, wherein the second linker is N-terminal of the interaction domain and wherein the third linker is C-terminal of the interaction domain. In some embodiments, the first linker, second linker, and/or third linker: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; comprises 3 repeating amino acid subunits or more; and/or comprises an amino acid sequence selected from the group comprising GSGSGSG (SEQ ID NO: 1), GGGGS (SEQ ID NO: 2), GSGSG (SEQ ID NO: 3), GGGG (SEQ ID NO: 4), GGG (SEQ ID NO: 5), GG (SEQ ID NO 6), GS (SEQ ID NO: 7), GSGS (SEQ ID NO: 8), GGGS (SEQ ID NO: 9), GGS (SEQ ID NO: 10), GTS (SEQ ID NO: 11), GGSGGS (SEQ ID NO: 12), GGG (SEQ ID NO: 13), GGGGGG (SEQ ID NO: 14), GGGGGGGGG (SEQ ID NO: 15), GGGGGGGGGGGG (SEQ ID NO: 16), GGGGGGGGGGGGGGG (SEQ ID NO: 17), GGS (SEQ ID NO: 18), GGSGGS (SEQ ID NO: 19), GGSGGSGGS (SEQ ID NO: 20), GGSGGSGGSGGS (SEQ ID NO: 21), GGSGGSGGSGGSGGS (SEQ ID NO: 22), GSG (SEQ ID NO: 23), GSGGSG (SEQ ID NO: 24), GSGGSGGSG (SEQ ID NO: 25), GSGGSGGSGGSG (SEQ ID NO: 26), GSGGSGGSGGSGGSG (SEQ ID NO: 27), (GGGGS), (SEQ ID NO: 28), GGGGSGGGGS (SEQ ID NO: 29), GGGGSGGGGSGGGGS (SEQ ID NO: 30), GGGGSGGGGSGGGGSGGGGS (SEQ ID NO: 31), and GGGGSGGGGSGGGGSGGGGSGGGGS (SEQ ID NO: 32).

In some embodiments, the Ca²⁺-sensing GvpC protein is capable of sensing Ca²⁺ dynamics at sub-micromolar concentrations with a sub-minute time scale. In some embodiments, the Ca²⁺-binding domain binding detectably binds Ca²⁺ with a dissociation constant (K_{d}) of less than about 1000 nM. In some embodiments, the Ca²⁺-sensing GV produces reversible non-linear ultrasound contrast in response to Ca²⁺ with half-maximal contrast occurring at less than about 1000 nM. In some embodiments, the Ca²⁺-sensing GV exhibits a detectable half-rise time of less than about 60 seconds upon contact with Ca²⁺. In some embodiments, the Ca²⁺-sensing GV exhibits a detectable half-decay time of less than about 60 seconds upon removal of Ca²⁺. In some embodiments, the Ca²⁺ affinity and/or response kinetics of the Ca²⁺-sensing GV are capable of being tuned by configuring one or more of the Ca²⁺-binding domain, the interaction domain, the first linker, the second linker, the third linker, and the insertion positions of the Ca²⁺-binding domain and the interaction domain within the parental GvpC protein.

In some embodiments, the Ca²⁺-sensing GvpC protein is derived from Anabaena flos-aquae (SEQ ID NO: 33), Halobacterium salinarum (SEQ ID NO: 34), Halobacterium mediterranei (SEQ ID NO: 35), Microchaete diplosiphon (SEQ ID NO: 36), Nostoc sp. (SEQ ID NO: 37), or a combination thereof. In some embodiments, the Ca²⁺-sensing GvpC protein comprises one or more truncation(s), insertion(s), and mutation(s) as compared to the parental GvpC protein from which it is derived. In some embodiments, the Ca²⁺-sensing GvpC protein comprises fewer tandem repeat regions than the parental GvpC protein. In some embodiments, the Ca²⁺-sensing GvpC protein comprises an amino acid sequence that is at least 80%, 85%, 90%, 95%, 98%, 99%, or 100% identical to any one of SEQ ID NOS: 38 and 43-45, or a portion thereof. In some embodiments, the nucleic acid composition comprises a DNA sequence that is at least 80%, 85%, 90%, 95%, 98%, 99%, or 100% identical to any one of SEQ ID NOS: 39-42, or a portion thereof. In some embodiments, the interaction domain comprises an amino acid sequence that is at least 80%, 85%, 90%, 95%, 98%, 99%, or 100% identical to any one of SEQ ID NOs: 46-51, or a portion thereof. In some embodiments, the Ca²⁺-binding domain comprises an amino acid sequence that is at least 80%, 85%, 90%, 95%, 98%, 99%, or 100% identical to any one of SEQ ID NOs: 52-58, or a portion thereof. In some embodiments, the Ca²⁺-binding domain comprises calmodulin (CaM) or a derivative thereof, optionally a mutant human CaM. In some embodiments, the interaction domain comprises or is derived from CaMKI. In some embodiments, the Ca²⁺-sensing GvpC protein comprises 3 tandem repeat regions, wherein the interaction domain is inserted within the second of the 3 tandem repeat regions. In some embodiments, the Ca²⁺-sensing GvpC protein is derived from a parental five tandem repeat GvpC protein natively expressed in Anabaena flos-aquae. In some embodiments, one or more of the mechanical, acoustic, surface and/or magnetic properties of the Ca²⁺-sensing GV are capable of being tuned by configuring one or more of the Ca²⁺-binding domain, the interaction domain, the first linker, the second linker, the third linker, and the insertion positions of the Ca²⁺-binding domain and the interaction domain within the parental GvpC protein.

In some embodiments, the first allosteric conformational change causes an at least about 1.1-fold reduction in the mechanical stiffness of Ca²⁺-sensing GV. In some embodiments, the fold reduction in mechanical stiffness is capable of being tuned by configuring one or more of the Ca²⁺-binding domain, the interaction domain, the first linker, the second linker, the third linker, and the insertion positions of the Ca²⁺-binding domain and the interaction domain within the parental GvpC protein. In some embodiments, the mechanical stiffness of a Ca²⁺-sensing GV in a GV stiff state is least about 1.1-fold greater than the mechanical stiffness of Ca²⁺-sensing GV in a GV soft state, and wherein a Ca²⁺-sensing GV in a GV soft state is capable of producing at least about 1.1-fold greater non-linear ultrasound signals as compared to a Ca²⁺-sensing GV in a GV stiff state. In some embodiments, a Ca²⁺-sensing GV in a GV soft state is capable of exhibiting an about 5 dB to about 50 dB enhancement in nonlinear ultrasound contrast as compared to a Ca²⁺-sensing GV in a GV stiff state. In some embodiments, the dynamic range of a Ca²⁺-sensing GV in a mammalian cell is about 5 dB to about 50 dB, optionally about 10-12 dB. In some embodiments, a Ca²⁺-sensing GV in a GV soft state is capable of exhibiting an at least about 1.1-fold increase in contrast to noise ratio (CNR) as compared to a Ca²⁺-sensing GV in a GV stiff state.

In some embodiments, the Ca²⁺-sensing GV in a GV soft state has a first buckling pressure profile. In some embodiments, the first buckling pressure profile comprises a buckling function from which a Ca²⁺-sensing GV in a GV soft state buckling amount can be determined for a given pressure value. In some embodiments, the buckling amount comprises the amount of nonlinear contrast. In some embodiments, the first buckling pressure profile comprises a first buckling threshold pressure where a Ca²⁺-sensing GV in a GV soft state starts to buckle and produce nonlinear contrast, a first optimum buckling pressure where a Ca²⁺-sensing GV in a GV soft state exhibits maximum buckling and produces the highest level of nonlinear contrast, a first collapse pressure wherein a Ca²⁺-sensing GV in a GV soft state collapses, any pressure between the first buckling threshold pressure and the first optimum buckling pressure, and any pressure between the first optimum buckling pressure and the first collapse pressure. In some embodiments, the Ca²⁺-sensing GV in a GV stiff state has a second buckling pressure profile. In some embodiments, the second buckling pressure profile comprises a buckling function from which a Ca²⁺-sensing GV in a GV stiff state buckling amount can be determined for a given pressure value. In some embodiments, the buckling amount comprises the amount of nonlinear contrast. In some embodiments, the second buckling pressure profile comprises a second buckling threshold pressure where a Ca²⁺-sensing GV in a GV stiff state starts to buckle and produce nonlinear contrast, a second optimum buckling pressure where a Ca²⁺-sensing GV in a GV stiff state exhibits maximum buckling and produces the highest level of nonlinear contrast, a second collapse pressure wherein a Ca²⁺-sensing GV in a GV stiff state collapses, any pressure between the second buckling threshold pressure and the second optimum buckling pressure, and any pressure between the second optimum buckling pressure and the second collapse pressure. In some embodiments, the first buckling pressure profile and the second buckling pressure profile are different. In some embodiments, the first buckling pressure profile and/or the second buckling pressure profile is capable of being tuned by configuring one or more of the Ca²⁺-binding domain, the interaction domain, the first linker, the second linker, the third linker, and the insertion positions of the Ca²⁺-binding domain and the interaction domain within the parental GvpC protein. In some embodiments, a selectable buckling pressure is the pressure value which produces the maximal difference in buckling between a Ca²⁺-sensing GV in a GV soft state and a Ca²⁺-sensing GV in a GV stiff state. In some embodiments, the selectable buckling pressure is: from about 40 kPa to about 1500 kPa; any collapse pressure within the first buckling pressure profile; any collapse pressure within the second buckling pressure profile; the first optimum buckling pressure; and/or the second optimum buckling pressure.

In some embodiments, the Ca²⁺-sensing GV in a GV soft state has a first collapse pressure profile. In some embodiments, the first collapse pressure profile comprises a collapse function from which a Ca²⁺-sensing GV in a GV soft state collapse amount can be determined for a given pressure value. In some embodiments, the first collapse pressure profile comprises a first initial collapse pressure where 5% or lower of a plurality of Ca²⁺-sensing GVs in a GV soft state are collapsed, a first midpoint collapse pressure where 50% of a plurality of Ca²⁺-sensing GVs in a GV soft state are collapsed, a first complete collapse pressure where at least 95% of a plurality of Ca²⁺-sensing GVs in a GV soft state are collapsed, any pressure between the first initial collapse pressure and the first midpoint collapse pressure, and any pressure between the first midpoint collapse pressure and the first complete collapse pressure. In some embodiments, a first selectable collapse pressure is: any collapse pressure within the first collapse pressure profile; selected from the first collapse pressure profile at a value between 0.05% collapse of a plurality of Ca²⁺-sensing GVs in a GV soft state and 95% collapse of a plurality of Ca²⁺-sensing GVs in a GV soft state; equal to or greater than the first initial collapse pressure; equal to or greater than the first midpoint collapse pressure; and/or equal to or greater than the first complete collapse pressure. In some embodiments, the Ca²⁺-sensing GV in a GV stiff state has a second collapse pressure profile. In some embodiments, the second collapse pressure profile comprises a collapse function from which a Ca²⁺-sensing GV in a GV stiff state collapse amount can be determined for a given pressure value. In some embodiments, the first collapse pressure profile and the second collapse pressure profile are different. In some embodiments, the first collapse pressure profile and/or second collapse pressure profile is capable of being tuned by configuring one or more of the Ca²⁺-binding domain, the interaction domain, the first linker, the second linker, the third linker, and the insertion positions of the Ca²⁺-binding domain and the interaction domain within the parental GvpC protein. In some embodiments, a midpoint of the second collapse profile has a higher pressure component than a midpoint of the first collapse profile. In some embodiments, the second collapse pressure profile comprises a second initial collapse pressure where 5% or lower of a plurality of Ca²⁺-sensing GVs in a GV stiff state are collapsed, a second midpoint collapse pressure where 50% of a plurality of Ca²⁺-sensing GVs in a GV stiff state are collapsed, a second complete collapse pressure where at least 95% of a plurality of Ca²⁺-sensing GVs in a GV stiff state are collapsed, any pressure between the second initial collapse pressure and the second midpoint collapse pressure, and any pressure between the second midpoint collapse pressure and the second complete collapse pressure. In some embodiments, a second selectable collapse pressure is: any collapse pressure within the second collapse pressure profile; selected from the second collapse pressure profile at a value between 0.05% collapse of a plurality of Ca²⁺-sensing GVs in a GV stiff state and 95% collapse of a plurality of Ca²⁺-sensing GVs in a GV stiff state; equal to or greater than the second initial collapse pressure; equal to or greater than the second midpoint collapse pressure; and/or equal to or greater than the second complete collapse pressure.

In some embodiments, the GVA gene(s) and/or GVS gene(s) are derived from a species of Anabaena bacteria, Halobacterium salinarum, and/or Bacillus megaterium. In some embodiments, one or more GV polynucleotides comprises: two or more GVS genes derived from different prokaryotic species; GVA genes and/or GVS genes from Bacillus Megaterium, Anabaena flos-aquae, Serratia sp., Burkholderia thailandensis, B. megaterium, Frankia sp, Haloferax mediterranei, Halobacterium sp, Microchaete diplosiphon, Nostoc sp, Halorubrum vacuolatum, Microcystis aeruginosa, Methanosarcina barkeri, Streptomyces coelicolor, and/or Psychromonas ingrahamii; gvpB, gvpN gvpF, gvpG, gvpL gvpS, gvpK, gvpJ, and/or gvpU from B. megaterium; gvpA, gvpN, gvpJ, gvpK, gvpF, gvpG, gvpV, and/or gvpW from Anabaena flos-aquae; gvpR, gvpN, gvpF, gvpG, gvpL, gvpS, gvpK, gvpJ, gvpT and/or gvpU from B. megaterium and gvpA from Anabaena flos-aquae; gvpA from Anabaena flos-aquae, and gvpN, gvpF, gvpG, gvpL, gvpS, gvpK, gvpJ, and/or gvpU from B. megaterium; and/or gvpA and/or gvpN from Anabaena flos-aquae, and gvpF, gvpG, gvpL, gvpS, gvpK, gvpJ, and/or gvpU from B. megaterium. In some embodiments, the GVA genes and/or GVS genes have sequences codon optimized for expression in a eukaryotic cell. In some embodiments, the Ca²⁺-sensing GV is a hybrid GV derived from two or more prokaryotic species.

In some embodiments, the one or more GV polynucleotide(s) comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element (e.g., woodchuck hepatitis post-translational regulatory element (WPRE), bovine growth hormone polyadenylation (bGH-polyA) signal sequence, human growth hormone polyadenylation (hGH-polyA) signal sequence, or any combination thereof). In some embodiments, one or more GV polynucleotides are operably connected to a promoter selected from the group comprising: a minimal promoter (e.g., TATA, miniCMV, and/or miniPromo); a ubiquitous promoter; a tissue-specific promoter and/or a lineage-specific promoter; and/or a ubiquitous promoter (e.g., cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin ((3-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

In some embodiments, nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon.

Disclosed herein include reporting therapeutic cells. In some embodiments, the reporting therapeutic cell comprises: Ca²⁺-sensing gas vesicles (GVs) disclosed herein, wherein the reporting therapeutic cell is configured to treat a disease or disorder of a subject upon administration. In some embodiments, the presence and/or functionality of the reporting therapeutic cells is capable of being monitored in vivo by application of ultrasound (US). In some embodiments, monitoring the functionality of the reporting therapeutic cells in vivo comprises detecting one or more Ca²⁺-coupled biological processes, optionally insulin secretion. In some embodiments, the reporting therapeutic cell is a replacement for a cell that is absent, diseased, infected, and/or involved in maintaining, promoting, or causing a disease or condition in a subject in need In some embodiments, the disease is a metabolic disease (e.g., T1D (type-1 diabetes), T2D (type-2 diabetes), diabetic ketoacidosis, obesity, cardiovascular disease, the metabolic syndrome and cancer). In some embodiments, the reporting therapeutic cell is autologous, allogenic, or xenogenic. In some embodiments, the reporting therapeutic cell is a stem cell (SC)-derived beta cell or a beta-mimetic designer cell. In some embodiments, the reporting therapeutic cell is capable of producing detectable ultrasound contrast in response to dynamic blood glucose levels and/or insulin secretion.

Disclosed herein include methods of generating reporting therapeutic cells. In some embodiments, the method comprises: introducing a nucleic acid composition disclosed herein into engineered therapeutic cells to generate reporting therapeutic cells capable of expressing the one or more GVA protein(s), the one or more GVS protein(s), and the Ca²⁺-sensing GvpC protein. In some embodiments, the engineered therapeutic cell is a stem cell (SC)-derived beta cell or a beta-mimetic designer cell.

Disclosed herein include methods of monitoring a cell-based therapy. In some embodiments, the method comprises: administering to a subject an effective amount of the reporting therapeutic cells disclosed herein; and applying ultrasound (US) to a target site of the subject, thereby monitoring the cell-based therapy. In some embodiments, the subject is a subject suffering from a disease or disorder In some embodiments, the disease is a metabolic disease (e.g., T1D (type-1 diabetes), T2D (type-2 diabetes), diabetic ketoacidosis, obesity, cardiovascular disease, the metabolic syndrome and cancer). In some embodiments, monitoring the cell-based therapy comprises monitoring the mass and/or function of the administered reporting therapeutic cells. In some embodiments, monitoring the functionality of the administered reporting therapeutic cells comprises detecting one or more Ca²⁺-coupled biological processes, optionally insulin secretion. In some embodiments, monitoring the cell-based therapy comprises determining the ratio of functional reporting therapeutic cells post-administration. In some embodiments, the reporting therapeutic cells are administered to a target site of the subject. In some embodiments, the administering comprises transplantation of the reporting therapeutic cells, optionally transplantation at one or more target sites. In some embodiments, the target site comprises a site of disease or disorder or is proximate to a site of a disease or disorder. In some embodiments, the cell-based therapy is a transplant and/or tissue replacement. In some embodiments, the administering comprises implanting the reporting therapeutic cells into a target site of the subject. In some embodiments, monitoring the cell-based therapy comprises monitoring blood glucose levels and/or insulin secretion at the target site.

Disclosed herein include extracellular signal-sensing cells. In some embodiments, the extracellular signal-sensing cell comprises: a sensing receptor capable of binding an extracellular signal, wherein sensor receptor signaling triggered by said binding is capable of modulating intracellular Ca²⁺ levels; and Ca²⁺-sensing gas vesicles (GVs) disclosed herein. In some embodiments, the extracellular signal-sensing cell is capable of producing detectable ultrasound contrast in response to dynamic extracellular signals. In some embodiments, the extracellular signal binding the sensing receptor is capable of activating a sensing receptor signaling pathway leading to calcium release from the endoplasmic reticulum of the extracellular signal-sensing cell, thereby elevating intracellular Ca²⁺ levels. In some embodiments, the sensing receptor is, comprises, or is derived from, a G protein-coupled receptor (GPCR).

In some embodiments, the GPCR is a chemokine receptor, a cytokine receptor, class A GPCR, class B GPCR, a class C GPCR, a class D GPCR, a class E GPCR, and a class F GPCR, adhesion GPCR, frizzled GPCR, acetylcholine receptor, melatonin receptor, melacortin receptor, motilin receptor, Lysophospholipid (LP A) receptor), adenosine receptor, adreno receptor, angiotensin receptor, bradykinin receptor, calcitonin receptor, calcium sensing receptor, cannabinoid receptor, chemokine receptor, cholecystokinin receptor, complement component (C5AR1) receptor, corticotrophin releasing factor receptor, dopamine receptor, endothelial differentiation gene receptor, endothelin receptor, formyl peptide-like receptor, galanin receptor, gastrin releasing peptide receptor, receptor ghrelin receptor, gastric inhibitory polypeptide receptor, glucagon receptor, gonadotropin releasing hormone receptor, histamine receptor, kisspeptin (KiSS1) receptor, leukotriene receptor, melanin-concentrating hormone receptor, melanocortin receptor, melatonin receptor, motilin receptor, neuropeptide receptor, nicotinic acid, opioid receptor, orexin receptor, orphan receptor, platelet activating factor receptor, prokineticin receptor, prolactin releasing peptide, prostanoid receptor, protease activated receptor, P2Y (purinergic) receptor, relaxin receptor, secretin receptor, serotonin receptor, somatostatin receptor, tachykinin receptor, vasopressin receptor, oxytocin receptor, vasoactive intestinal peptide (VIP) receptor or the pituitary adenylate cyclase activating polypeptide (PACAP) receptor, taste 1 receptor, metabotropic glutamate receptor, calcium-sensing receptor, brain specific angiogenesis inhibitor receptor (1, 2 or 3), cadherein receptor, an estrogen receptor, or any combination thereof. In some embodiments, the extracellular signal is one or more of a polypeptide, a peptide, a nucleotide, a growth factor, ahormone, a pheromone, a chemokine, a cytokine, a neurotransmitter, a lipid, and a sugar. In some embodiments, the hormone is selected from the group comprising thyroid-stimulating hormone, a follicle-stimulating hormone, a leuteinizing hormone, prolactin, growth hormone, adrenocorticotropic hormone, antidiuretic hormone, oxytocin, thyrotropin-releasing hormone, gonadotropin-releasing hormone, growth hormone-releasing hormone, corticotropin-releasing hormone, somatostatin, dopamine, melatonin, thyroxine, calcitonin, parathyroid hormone, a glucocorticoid, a mineralocorticoid, an androgen, adrenaline, an estrogen, progesterone, human chorionic gonadotropin, insulin, glucagons, somatostatin, erythropoietin, calcitriol, atrial-natriuretic peptide, gastrin, secretin, cholecystokinin, somatostatin, neuropeptide Y, ghrelin, PYY3.36, insulin-like growth factor-1, angiotensinogen, thrombopoietin, leptin, or any combination thereof. In some embodiments, the neurotransmitter is selected from the group comprising acetylcholine, epinephrine (adrenaline), norepinephrine (noradrenaline), dopamine, 5-hydroxytryptamine (serotonin), glutamic acid, L-3,4-dihydroxyphenylalanine (L-dopa), 3,4-Dihydroxyphenylacetic acid (DOPAC), homovannilic acid, tyramine, or any combination thereof. In some embodiments, the neurotransmitter is a neuroactive peptide (e.g., bradykinin, cholecystokinin, gastrin, secretin, oxytocin, a sleep peptide, gonadotropin-releasing hormone, beta-endorphin, enkephalin, substance P, somatostatin, prolactin, galanin, growth hormone-releasing hormone, bombesin, dynorphin, neurotensin, motilin, thyrotropin, neuropeptide Y, leuteinizing hormone, calcitonin, or vasoactive intestinal peptide, or any combination thereof).

In some embodiments, the extracellular signal-sensing cell is capable of dynamically sensing extracellular signals upon administration to a subject. In some embodiments, said administration comprises transplantation, optionally transplantation into one or more target brain region(s). In some embodiments, the extracellular signal-sensing cell comprises one or more secondary sensing receptor(s), wherein the sensor receptor and the secondary sensing receptor(s) target different agonists and/or bind different extracellular signals. In some embodiments, the extracellular signal-sensing cell is capable of dynamically sensing changes in the concentration of the extracellular signal via changes in nonlinear ultrasound contrast. In some embodiments, the extracellular signal-sensing cells are implanted within a target site of a subject, and wherein applying US comprises applying US to the target site to obtain a US image of the target site. In some embodiments, an at least about 5 dB, about 4 dB, about 3 dB, about 2 dB, about 1 dB, about 0.8 dB, about 0.6 dB, about 0.4 dB, about 0.2 dB, about 0.1 dB, about 0.05 dB, or about 0.01 dB, enhancement in nonlinear ultrasound contrast indicates the presence of the extracellular signal. In some embodiments, administration of the effective amount of extracellular signal-sensing cells to a subject does not cause neurological side effects, toxicity, and/or disruption of physiological functions of the subject. In some embodiments, in the absence of the extracellular signal, the sensing receptor has low or no endogenous activity. In some embodiments, the extracellular signal-sensing cell has low endogenous signaling of the sensing receptor, optionally less than 10 percent of maximal signaling of the sensing receptor upon binding the extracellular signal. In some embodiments, the amount of ultrasound contrast is correlated with the amount of extracellular signal at the target site.

Disclosed herein include methods of monitoring extracellular signal dynamics. In some embodiments, the method comprises: administering to a subject an effective amount of the extracellular signal-sensing cells disclosed herein; and applying ultrasound (US) to a target site of the subject, thereby monitoring extracellular signal dynamics. In some embodiments, applying US causes the Ca²⁺-sensing GVs to produce an extracellular signal-dependent nonlinear ultrasound contrast.

Disclosed herein include methods of monitoring a cell-based therapy. In some embodiments, the method comprises: administering to a subject an effective amount of the extracellular signal-sensing cells disclosed herein; administering to the subject an effective amount of a cell-based therapy; and applying ultrasound (US) to a target site of the subject, thereby monitoring the cell-based therapy.

In some embodiments, the performance of the cell-based therapy is associated with extracellular signal dynamics. In some embodiments, monitoring the cell-based therapy comprises monitoring the viability and/or functionality of the cell-based therapy via monitoring extracellular signal dynamics. In some embodiments, the viability and/or functionality of the cell-based therapy is associated with and/or caused by extracellular signal dynamics. In some embodiments, monitoring the cell-based therapy comprises monitoring the microenvironment of the cell-based therapy via monitoring extracellular signal dynamics. In some embodiments, the extracellular signal-sensing cells and/or cell-based therapy are administered to a target site of the subject. In some embodiments, the extracellular signal-sensing cells and cell-based therapy are co-administered. In some embodiments, the extracellular signal-sensing cells are incorporated into the cell-based therapy. In some embodiments, the administering comprises transplantation, optionally transplantation at one or more target sites. In some embodiments, the target site comprises a site of disease or disorder or is proximate to a site of a disease or disorder, optionally the target site(s) comprises one or more target brain region(s). In some embodiments, the cell-based therapy is a transplant and/or tissue replacement.

Disclosed herein include methods of monitoring a neural circuit of the subject. In some embodiments, the method comprises: administering to a subject an effective amount of the extracellular signal-sensing cells disclosed herein; and applying ultrasound (US) to a target site of the subject, thereby monitoring a neural circuit of the subject. In some embodiments, the activity of the neural circuit is caused by and/or associated with extracellular signal dynamics and/or Ca²⁺-associated biological processes. In some embodiments, the neural circuit is associated with a disease or disorder, e.g., one or more of schizophrenia, drug craving, drug addiction, bipolar disorder, anxiety, depression, Parkinson's disease, Alzheimer's disease, cognitive dysfunction, multiple sclerosis, Amyotrophic lateral sclerosis (ALS), ischemic stroke, HIV dementia, and Huntington's disease. In some embodiments, the neural circuit is associated with a behavior or physiological function, e.g., one or more of learning, motivation, memory, attention, concentration, alertness, mental flexibility and/or speed, learning, intelligence, language skills, problem solving capacity, consciousness, coping with psychological stress or tension, motivation, mobility, decision making capacity, reaction time, and regulation of emotions.

Disclosed herein include methods of monitoring a physiological function of a subject. In some embodiments, the method comprises: administering to a subject an effective amount of the extracellular signal-sensing cells disclosed herein; and applying ultrasound (US) to a target site of the subject, thereby monitoring the physiological function of the subject. In some embodiments, the physiological function is caused by and/or associated with extracellular signal dynamics and/or Ca²⁺-associated biological processes. In some embodiments, the physiological function comprises an immune function or metabolic function of the subject.

Disclosed herein include methods of identifying or monitoring a disease or disorder. In some embodiments, the method comprises: administering to a subject an effective amount of the extracellular signal-sensing cells disclosed herein; and applying ultrasound (US) to a target site of the subject, thereby identifying or monitoring the disease or disorder. In some embodiments, the disease or disorder is caused by and/or associated with extracellular signal dynamics and/or Ca²⁺-associated biological processes.

In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. In some embodiments, the disease or disorder comprises a neurological disease or disorder.

In some embodiments, the neurological disease or disorder comprises epilepsy, Dravet Syndrome, Lennox Gastaut Syndrome, mycolonic seizures, juvenile mycolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof. In some embodiments, applying US comprises applying hemodynamic functional US.

Disclosed herein include methods of generating Ca²⁺-sensing gas vesicles (GVs). In some embodiments, the method comprises: introducing a nucleic acid composition disclosed herein into host cells capable of expressing the one or more GVA protein(s), the one or more GVS protein(s), and the Ca²⁺-sensing GvpC protein to generate the Ca²⁺-sensing gas vesicles (GVs). In some embodiments, the host cell is a prokaryotic cell or a eukaryotic cell.

Disclosed herein include methods of generating Ca²⁺-sensing gas vesicles (GVs). In some embodiments, the method comprises: providing gas vesicles (GVs) comprising a naturally occurring GvpC protein; removing the naturally occurring GvpC protein from said GVs to generate stripped GVs, optionally via urea; and contacting the stripped GVs with a Ca²⁺-sensing GvpC protein disclosed herein, thereby generating Ca²⁺-sensing gas vesicles (GVs).

Disclosed herein include methods of imaging a target site of a subject. In some embodiments, the method comprises: obtaining a subject comprising the Ca²⁺-sensing gas vesicles (GVs) disclosed herein; and applying ultrasound (US) to the target site of the subject to obtain a US image of the target site, optionally a nonlinear US image. In some embodiments, the obtaining comprises introducing a nucleic acid composition disclosed herein into one or more target cells of said subject.

Disclosed herein include methods of imaging intracellular Ca²⁺ dynamics. In some embodiments, the method comprises: obtaining target cells comprising the Ca²⁺-sensing gas vesicles (GVs) disclosed herein; and applying ultrasound (US) to said target cells, thereby imaging intracellular Ca²⁺ dynamics. In some embodiments, the obtaining comprises introducing a nucleic acid composition disclosed herein into the target cells, optionally target cells of a subject.

Disclosed herein include methods of monitoring Ca²⁺-associated biological processes. In some embodiments, the method comprises: obtaining target cells comprising the Ca²⁺-sensing gas vesicles (GVs) disclosed herein; and applying ultrasound (US) to said target cells, thereby monitoring Ca²⁺-associated biological processes. In some embodiments, the obtaining comprises introducing a nucleic acid composition disclosed herein into the target cells, optionally target cells of a subject.

Disclosed herein include methods of detecting perturbation-induced changes in intracellular Ca²⁺ dynamics. In some embodiments, the method comprises: obtaining target cells comprising the Ca²⁺-sensing gas vesicles (GVs) disclosed herein; introducing one or more genetic, chemical, and/or physical perturbations to said target cells; and applying ultrasound (US) to said target cells, thereby detecting the perturbation-induced changes in intracellular Ca²⁺ dynamics.

In some embodiments, the obtaining comprises introducing a nucleic acid composition disclosed herein into the target cells, optionally target cells of a subject. In some embodiments, target cells are situated within a target site of a subject, and wherein applying US comprises applying US to the target site to obtain a US image of the target site. In some embodiments, the target cells are situated within a plurality of target sites of a subject, and wherein applying US comprises applying US to the plurality of target sites to obtain US images of the plurality of target sites. In some embodiments, applying US to the target site comprises applying US to a plurality of target sites of a subject. In some embodiments, applying US causes the Ca²⁺-sensing GVs to produce a Ca²⁺-dependent nonlinear ultrasound contrast. In some embodiments, an at least about 5 dB, about 4 dB, about 3 dB, about 2 dB, about 1 dB, about 0.8 dB, about 0.6 dB, about 0.4 dB, about 0.2 dB, about 0.1 dB, about 0.05 dB, or about 0.01 dB, enhancement in nonlinear ultrasound contrast indicates the presence of intracellular Ca²⁺ and/or a Ca²⁺-associated biological processes.

In some embodiments, applying US comprises nonlinear US imaging. In some embodiments, applying US comprises applying one or more US pulse(s) over a duration of time. In some embodiments, the duration of time is about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the one or more US pulse(s) each have a pulse duration of about 1 hour, about 30 minutes, about 15 minutes, about 10 minutes, about 5 minutes, about 1 minute, about 1 second, or about 1 millisecond. In some embodiments, applying one or more US pulse(s) comprises applying one or more focused US pulse(s). In some embodiments, applying one or more US pulse(s) comprises applying US at a frequency of 100 kHz to 100 MHz.

In some embodiments, applying one or more US pulse(s) comprises applying ultrasound having a mechanical index in a range between 0.2 and 0.6. In some embodiments, the one or more US pulse(s) comprise a peak pressure of about 40 kPa to about 1500 kPa. In some embodiments, the one or more US pulse(s) comprise a pressure value that is: the selectable buckling pressure; the first optimum buckling pressure; and/or selected from the first buckling pressure profile that optimally maximizes buckling of the Ca²⁺-sensing GV in a GV soft state while minimizing buckling of Ca²⁺-sensing GV in a GV stiff state. In some embodiments, the one or more US pulse(s) induces collapse of Ca²⁺-sensing GV in a GV soft state, and wherein the one or more US pulse(s) comprise a pressure value that is: the first selectable collapse pressure; the second selectable collapse pressure; and/or selected from the second collapse pressure profile that optimally maximizes collapse of the Ca²⁺-sensing GV in a GV soft state while minimizing collapse of Ca²⁺-sensing GV in a GV stiff state.

In some embodiments, the nonlinear ultrasound imaging comprises cross-amplitude modulation (x-AM) ultrasound imaging or parabolic amplitude modulation (pAM) ultrasound imaging. In some embodiments, the nonlinear ultrasound imaging comprises differential nonlinear ultrasound imaging. In some embodiments, differential nonlinear ultrasound imaging comprises imaging of the second and/or higher harmonics with the first harmonic signal subtracted out. In some embodiments, the method comprises cross-phase modulation imaging and/or harmonic imaging. In some embodiments, the nonlinear ultrasound imaging comprises providing amplitude modulation (AM) ultrasound pulse sequences in order to image and differentiate the baseline nonlinear behavior of buckling Ca²⁺-sensing GV in a GV stiff state from the increased nonlinear behavior of buckling Ca²⁺-sensing GV in a GV soft state. In some embodiments, the nonlinear ultrasound imaging comprises: pairs of cross-propagating plane waves to elicit nonlinear scattering from buckling Ca²⁺-sensing GVs at the wave intersection; subtracting the signal generated by transmitting each wave on its own; and quantifying the resulting contrast. In some embodiments, the signals generated by transmitting each wave on its own has linear characteristics and/or lower nonlinear characteristics than the combined transmission of both plane waves produced at their intersection. In some embodiments, the nonlinear ultrasound imaging comprises: a peak positive pressure of two single tilted plane waves exciting the Ca²⁺-sensing GV in a linear scattering regime; a doubled X-wave intersection amplitude exciting the Ca²⁺-sensing GV in a nonlinear scattering regime; summing the echoes from the two single tilted plane-wave transmissions to generate a sum; and subtracting the sum from the echoes of the X-wave transmissions to derive nonzero differential Ca²⁺-sensing GV signals.

In some embodiments, applying US comprises detecting scattering of the one or more US pulse(s) by Ca²⁺-sensing GV. In some embodiments, applying US comprises detecting increased nonlinear scattering of the US by buckling Ca²⁺-sensing GV in a GV soft state. In some embodiments, detecting scattering comprises: detecting backscattered echoes of two half-amplitude transmissions at applied pressures below the buckling threshold of the Ca²⁺-sensing GV. In some embodiments, said two half-amplitude transmissions trigger largely linear scattering. In some embodiments, detecting backscattered echoes of a third full-amplitude transmission at pressures above the buckling threshold of the Ca²⁺-sensing GV. In some embodiments, said third full-amplitude transmission triggers harmonic and nonlinear scattering. In some embodiments, the method comprises subtracting the backscattered echoes of the two half-amplitude transmissions from the backscattered echoes of the third full-amplitude transmission.

In some embodiments, the method comprises: single-cell Ca²⁺ imaging; and/or imaging a large volume in deep tissue. In some embodiments, the method comprises US imaging with a spatiotemporal resolution of less than about 100 μm and less than about 1 ms. In some embodiments, the target site comprises: a volume larger than about 1 mm³; a depth deeper than about 1 mm; a depth and/or a volume inaccessible via optical imaging and/or fiber photometry; and/or the entire brain or a portion thereof. In some embodiments, the target site comprises target cells. In some embodiments, the target cells comprise excitable cells, optionally the biological processes comprise one or more of Ca²⁺-coupled neural activities, synaptic plasticity, and brain electrical signaling. In some embodiments, the target cells comprise non-excitable cells, optionally the biological processes comprise T-cell activation and insulin secretion from beta cells. In some embodiments, the target cells are in vitro, in vivo, and/or ex vivo, optionally the target cells are tissue culture cells. In some embodiments, the subject is a mammal. In some embodiments, the subject is not anesthetized. In some embodiments, the target site comprises a site of disease or disorder or is proximate to a site of a disease or disorder.

In some embodiments, the target site comprises a tissue, optionally the tissue is inflamed tissue and/or infected tissue. In some embodiments, the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the tissue comprises: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue.

In some embodiments, the target site comprises one or more target brain region(s), and wherein the target brain region(s) comprises the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (51), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof.

In some embodiments, the brain region comprises neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof.

### BRIEF DESCRIPTION OF THE DRAWINGS

FIGS. 1A-1D depict non-limiting exemplary schematics and data related to gas vesicles as acoustic reporter genes and biosensors. FIG. 1A depicts a TEM image of a GV; scale bar 100 nm. FIG. 1B depicts a diagram of GV structure. A 2 nm-thick protein shell encloses an air-filled compartment. FIG. 1C depicts engineered gene clusters enabling GV expression in mammalian cells. FIG. 1D depicts a schematic showing ultrasound imaging of GV expression in mouse tumor xenograft. Heat colormap shows GV-specific non-linear contrast. Grayscale image shows anatomical background. Scale bar 1 mm.

FIGS. 2A-2F depict non-limiting exemplary schematics and data related to UGECI design and in vitro characterization. FIG. 2A depicts a finite element model of buckling and non-buckling GVs. FIG. 2B depicts a schematic related to the structural role of GvpC. FIG. 2C depicts a schematic related to the design of calcium-sensing GvpC. FIG. 2D depicts a schematic showing Ca²⁺ binding results in GvpC softening, GV buckling and non-linear ultrasound contrast.

FIG. 2E depicts a nonlinear ultrasound image of agarose-embedded UGECIs incubated without Ca²⁺, with 100 μM Ca²⁺, and following the addition of 10 mM EGTA. Scale bar 1 mm. FIG. 2F depicts data related to normalized nonlinear ultrasound contrast as a function of free Ca²⁺.

FIG. 2G depicts data related to time traces of UGECIs mixed into 14 uM Ca²⁺ (orange) and UGECI pre-inducted with 100 uM Ca²⁺ mixed into 10 mM EGTA (grey). FIG. 2H depicts a diagram of the non-limiting exemplary modifiable components of GvpC in the UGECI design. N=3. Error bars and shade indicate SEM, and are only displayed when larger than the symbols. Bars and solid lines indicate mean for (FIG. 2E, FIG. 2G) and solid line indicates fitted curve for (FIG. 2F). Color bars and CNR unit: dB. All experiments were done in 37° C.

FIGS. 3A-3D depict data related to UGECI expression and characterization in HEK293T cells. FIG. 3A depicts a nonlinear ultrasound image of agarose-embedded HEK293T cells expressing UGECI or control construct incubated with 0.05% saponin and 2 mM Ca²⁺ or 5 mM EGTA. FIG. 3B depicts a bar graph showing the ultrasound CNR of cells expressing UGECI or control construct incubated with 0.05% saponin and 2 mM Ca²⁺ or 5 mM EGTA. FIG. 3C depicts a nonlinear ultrasound image of agarose-embedded HEK293T cells expressing UGECI or control construct incubated with 10 μM ionomycin and 2 mM Ca²⁺ or 5 mM EGTA. FIG. 3D depicts a bar graph showing the ultrasound CNR of cells expressing UGECI or control construct incubated with 10 μM ionomycin and 2 mM Ca²⁺ or 5 mM EGTA. Scale bar 1 mm. N=5 for (FIG. 3B) and N=3 for (FIG. 3D). Error bars indicate SEM and bars indicate mean. Color bars and CNR unit: dB. All experiments were done in 37° C.

FIGS. 4A-4C depict data related to UGECI in vitro characterization. FIG. 4A depicts data related to normalized gas vesicle stiffness as a function of free Ca²⁺. N=1. FIG. 4B depicts data related to normalized nonlinear ultrasound contrast as a function of free Ca²⁺. N=3. Error bars indicate SEM, and are only displayed when larger than the symbols. FIG. 4C depicts data related to time traces of UGECI-241 mixed into 14 uM Ca²⁺ (orange) and UGECI-241 pre-inducted with 100 uM Ca²⁺ mixed into 10 mM EGTA (gray). Dots=individual data points. N=3 with 3 technical replicates for each biological replica. All the solid lines indicate fitted curves. All experiments were done in 37° C.

FIGS. 5A-5B depicted non-limiting exemplary schematics related to cell-based ultrasonic biosensors of extracellular signals (CUBES) (e.g., extracellular signal-sensing cells) provided herein. FIG. 5A depicts an illustration of CUBES implanted at several locations in the brain, where they can sense dynamic extracellular signals and produce noninvasively detectable ultrasound contrast. FIG. 5B depicts the design of neurotransmitter-sensing CUBES in which the neurotransmitter activates a GPCR pathway leading to calcium release from the endoplasmic reticulum, which is converted into ultrasound contrast by genetically encoded acoustic biosensors of calcium.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

There are provided, in some embodiments, nucleic acid compositions. The nucleic acid composition can comprise: one or more promoters operably connected to one or more gas vesicle (GV) polynucleotides comprising: a Ca²⁺-sensing GvpC gene encoding a Ca²⁺-sensing GvpC protein. In some embodiments, the Ca²⁺-sensing GvpC protein is capable of associating with a gas vesicle structural (GVS) protein to form a Ca²⁺-sensing gas vesicle (GV).

There are provided, in some embodiments, nucleic acid compositions. The nucleic acid composition can comprise: one or more promoters operably connected to one or more gas vesicle (GV) polynucleotides comprising: one or more gas vesicle assembly (GVA) gene(s) encoding one or more GVA protein(s), one or more gas vesicle structural (GVS) gene(s) encoding one or more GVS protein(s) selected from GvpA and GvpB, and/or a Ca²⁺-sensing GvpC gene encoding a Ca²⁺-sensing GvpC protein. In some embodiments, the one or more GVA protein(s), the one or more GVS protein(s), and the Ca²⁺-sensing GvpC protein are capable of forming Ca²⁺-sensing gas vesicles (GVs) upon expression in a cell or a cell-like environment.

Disclosed herein include compositions. In some embodiments, the composition comprises: a Ca²⁺-sensing GvpC protein, wherein the Ca²⁺-sensing GvpC protein is capable of associating with a gas vesicle structural (GVS) protein to form a Ca²⁺-sensing gas vesicle (GV).

Disclosed herein include compositions. In some embodiments, the composition comprises: a Ca²⁺-sensing gas vesicle (GV) comprising a Ca²⁺-sensing GvpC protein and one or more GVS protein(s) selected from GvpA and GvpB.

Disclosed herein include reporting therapeutic cells. In some embodiments, the reporting therapeutic cell comprises: Ca²⁺-sensing gas vesicles (GVs) disclosed herein, wherein the reporting therapeutic cell is configured to treat a disease or disorder of a subject upon administration.

Disclosed herein include methods of generating reporting therapeutic cells. In some embodiments, the method comprises: introducing a nucleic acid composition disclosed herein into engineered therapeutic cells to generate reporting therapeutic cells capable of expressing the one or more GVA protein(s), the one or more GVS protein(s), and the Ca²⁺-sensing GvpC protein. In some embodiments, the engineered therapeutic cell is a stem cell (SC)-derived beta cell or a beta-mimetic designer cell.

Disclosed herein include methods of monitoring a cell-based therapy. In some embodiments, the method comprises: administering to a subject an effective amount of the reporting therapeutic cells disclosed herein; and applying ultrasound (US) to a target site of the subject, thereby monitoring the cell-based therapy.

Disclosed herein include extracellular signal-sensing cells. In some embodiments, the extracellular signal-sensing cell comprises: a sensing receptor capable of binding an extracellular signal, wherein sensor receptor signaling triggered by said binding is capable of modulating intracellular Ca²⁺ levels; and Ca²⁺-sensing gas vesicles (GVs) disclosed herein.

Disclosed herein include methods of monitoring extracellular signal dynamics. In some embodiments, the method comprises: administering to a subject an effective amount of the extracellular signal-sensing cells disclosed herein; and applying ultrasound (US) to a target site of the subject, thereby monitoring extracellular signal dynamics.

Disclosed herein include methods of monitoring a cell-based therapy. In some embodiments, the method comprises: administering to a subject an effective amount of the extracellular signal-sensing cells disclosed herein; administering to the subject an effective amount of a cell-based therapy; and applying ultrasound (US) to a target site of the subject, thereby monitoring the cell-based therapy.

Disclosed herein include methods of monitoring a neural circuit of the subject. In some embodiments, the method comprises: administering to a subject an effective amount of the extracellular signal-sensing cells disclosed herein; and applying ultrasound (US) to a target site of the subject, thereby monitoring a neural circuit of the subject.

Disclosed herein include methods of monitoring a physiological function of a subject. In some embodiments, the method comprises: administering to a subject an effective amount of the extracellular signal-sensing cells disclosed herein; and applying ultrasound (US) to a target site of the subject, thereby monitoring the physiological function of the subject.

Disclosed herein include methods of identifying or monitoring a disease or disorder. In some embodiments, the method comprises: administering to a subject an effective amount of the extracellular signal-sensing cells disclosed herein; and applying ultrasound (US) to a target site of the subject, thereby identifying or monitoring the disease or disorder.

Disclosed herein include methods of generating Ca²⁺-sensing gas vesicles (GVs). In some embodiments, the method comprises: introducing a nucleic acid composition disclosed herein into host cells capable of expressing the one or more GVA protein(s), the one or more GVS protein(s), and the Ca²⁺-sensing GvpC protein to generate the Ca²⁺-sensing gas vesicles (GVs). In some embodiments, the host cell is a prokaryotic cell or a eukaryotic cell.

Disclosed herein include methods of generating Ca²⁺-sensing gas vesicles (GVs). In some embodiments, the method comprises: providing gas vesicles (GVs) comprising a naturally occurring GvpC protein; removing the naturally occurring GvpC protein from said GVs to generate stripped GVs, optionally via urea; and contacting the stripped GVs with a Ca²⁺-sensing GvpC protein disclosed herein, thereby generating Ca²⁺-sensing gas vesicles (GVs).

Disclosed herein include methods of imaging a target site of a subject. In some embodiments, the method comprises: obtaining a subject comprising the Ca²⁺-sensing gas vesicles (GVs) disclosed herein; and applying ultrasound (US) to the target site of the subject to obtain a US image of the target site, optionally a nonlinear US image. In some embodiments, the obtaining comprises introducing a nucleic acid composition disclosed herein into one or more target cells of said subject.

Disclosed herein include methods of imaging intracellular Ca²⁺ dynamics. In some embodiments, the method comprises: obtaining target cells comprising the Ca²⁺-sensing gas vesicles (GVs) disclosed herein; and applying ultrasound (US) to said target cells, thereby imaging intracellular Ca²⁺ dynamics. In some embodiments, the obtaining comprises introducing a nucleic acid composition disclosed herein into the target cells, optionally target cells of a subject.

Disclosed herein include methods of monitoring Ca²⁺-associated biological processes. In some embodiments, the method comprises: obtaining target cells comprising the Ca²⁺-sensing gas vesicles (GVs) disclosed herein; and applying ultrasound (US) to said target cells, thereby monitoring Ca²⁺-associated biological processes. In some embodiments, the obtaining comprises introducing a nucleic acid composition disclosed herein into the target cells, optionally target cells of a subject.

Disclosed herein include methods of detecting perturbation-induced changes in intracellular Ca²⁺ dynamics. In some embodiments, the method comprises: obtaining target cells comprising the Ca²⁺-sensing gas vesicles (GVs) disclosed herein; introducing one or more genetic, chemical, and/or physical perturbations to said target cells; and applying ultrasound (US) to said target cells, thereby detecting the perturbation-induced changes in intracellular Ca²⁺ dynamics.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

The term “brain cell” as used herein shall be given its ordinary meaning and shall also refer to cells that form the brain of an individual with the exclusion of blood vessels and meninges (dura mater, arachnoid mater, and pia mater in mammals) of the individual. Exemplary brain cells comprise neurons and glial cells.

The terms “neuron”, “nerve cell or “neural cell” as used herein interchangeably shall be given their ordinary meaning and also indicate an electrically excitable cell that receives, processes, and transmits information through electrical and chemical signals. A neuron consists of a cell body (or soma) which contains the neuron's nucleus (with DNA and typical nuclear organelles), branching dendrites (signal receivers) and a projection called axon, which take information away from the cell body and conduct the nerve signal. At the other end of the axon, axon terminals transmit the electro-chemical signal across a synapse (the gap between the axon terminal and the receiving cell). Accordingly, neural brain cells are nerve cells of the brain that transmit nerve signals to and from the brain.

Brain cells are comprised within areas of the brain defined as gray matter and white matter. The gray matter indicates an area of the brain comprising primarily neuronal cell bodies, neuropil (dendrites and myelinated as well as unmyelinated axons), glial cells (astrocytes and microglia), and synapses. White matter indicates an area of the brain which mainly comprise myelinated axons, also called tracts.

Brain cells are also comprised within “brain regions” which are areas anatomically defined by appearance and position as well as by their locations and their relationships with other parts of the brain. Exemplary brain regions can comprise the medulla (region containing many small nuclei involved in a wide variety of sensory and involuntary motor functions such as vomiting, heart rate and digestive processes), the pons (region of the brainstem directly above the medulla, which contains nuclei that control often voluntary but simple acts such as sleep, respiration, swallowing, bladder function, equilibrium, eye movement, facial expressions, and posture, includes) the hypothalamus (small region at the base of the forebrain composed of numerous small nuclei, each with distinct connections and neurochemistry, and engaged in additional involuntary or partially voluntary acts such as sleep and wake cycles, eating and drinking, and the release of some hormones), the thalamus (a region of nuclei with diverse functions such as relaying information to and from the cerebral hemispheres, motivation, and action-generating systems such as the action generating systems for several types of “consummatory” behaviors such as eating, drinking, defecation, and copulation, in the subthalamic area also zona incerta), the cerebellum (a region modulating the outputs of other brain regions, whether motor related or thought related, to make them certain and precise), the optic tectum (a region usually referred to as the superior colliculus in mammals, allowing actions such as eye movements and reaching movements to be directed toward points in space, most commonly in response to visual input), the pallium (a region of gray matter that lies on the surface of the forebrain also identified in reptiles and mammals as cerebral cortex which with multiple functions including smell and spatial memory), the hippocampus, (a region involved in complex events such as spatial memory and navigation in fishes, birds, reptiles, and mammals), the basal ganglia (a region involved in action selection as the related brain cells send inhibitory signals to all parts of the brain that can generate motor behaviors, and in the right circumstances release the inhibition, it comprises regions such as caudate nucleus, putamen, globus pallidus, substantia nigra, subthalamic nucleus, nucleus accumbens) and the olfactory bulb (a region that processes olfactory sensory signals and sends its output to the olfactory part of the pallium.

In some embodiments brain cells are further comprised in neural circuits possibly comprising cells and regions of additional parts of the body including cells of the peripheral nervous systems and other systems and organs of the body of the individual.

The wording “neural circuits” shall be given its ordinary meaning and shall also refer to a population of cells including neurons interconnected by synapses to pass an electrochemical signal from a neuron to another to carry out a specific function when activated. In some embodiments, the specific function neural circuits herein described manifests in a behavior or physiological function of the individual.

The term “behavior” as used herein shall be given its ordinary meaning and shall also refer to an internally coordinated responses (actions or inactions) of a whole living individual to internal and/or external stimuli. Exemplary behaviors in the sense of the disclosure comprise eating, drinking, defecation, and copulation, speaking, contemplating, remembering, focusing attention and additional behaviors identifiable by a skilled person.

The wording “physiological function” as used herein shall be given its ordinary meaning and shall also refer to a series of action and reactions performed by components of a living organism such as organ systems, organs, cells, and biomolecules to carry out the chemical and physical functions that exist in the living system. Exemplary physiological functions comprise action and reactions performed by components of the organism of an individual to carry out digestion of food, circulation of blood, contraction of muscles as well as other biophysical and biochemical phenomena, related to the coordinated homeostatic control mechanisms, and the continuous communication between cells in a living organism.

Neural circuits control behaviors and physiological function of an individual and changes in activity of neural circuits can lead to changes in behaviors and physiological functions of an individual as will be understood by a skilled person.

Exemplary neural circuit comprise the trisynaptic circuit in the hippocampus. the Papez circuit linking the hypothalamus to the limbic lobe, and neural circuits in the cortico-basal ganglia-thalamo-cortical loop which transmit information from the cortex, to basal ganglia, and thalamus, and back to the cortex, as well as the microcircuitry internal to the striatum the largest structure within the basal ganglia and additional circuits identifiable by a skilled person.

As used herein, the terms “nucleic acid” and “polynucleotide” are interchangeable and can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “plasmid” can refer to a nucleic acid that can be used to replicate recombinant DNA sequences within a host organism. The sequence can be a double stranded DNA.

The term “element” can refer to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “enhancer” refers to a type of regulatory element that can increase the efficiency of transcription, regardless of the distance or orientation of the enhancer relative to the start site of transcription.

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “variant” can refer to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. In some embodiments, the subject is a rodent (e.g., rat or mouse). In some embodiments, the subject is a primate (e.g., human or money).

As used herein, the term “effective amount” refers to an amount sufficient to effect beneficial or desirable imaging, biological, diagnostic, and/or clinical results.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The term “autologous” shall be given its ordinary meaning, and shall also refer to any material derived from the same individual to whom it is later to be re-introduced into the individual.

The term “allogeneic” shall be given its ordinary meaning, and shall also refer to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

### Ultrasonic Genetically Encoded Calcium Indicators

There are provided, in some embodiments, compositions and methods for calcium imaging solving the aforementioned problems in the art. Ultrasound-based methods and compositions provided herein can possess a number of unique advantages for deep tissue calcium imaging, such as, for example, safety, inexpensive and widely compatible equipments, and, ability to penetrate much deeper than light (several cm) while providing relatively high spatiotemporal resolution (<100 μm and 1 ms) and large imaging volume (˜cm³). Disclosed herein include methods for deep tissue calcium imaging that employ the first ultrasonic genetically encoded calcium indicators (UGECIs) (e.g., Ca²⁺-sensing GvpC proteins, Ca²⁺-sensing gas vesicles), disclosed herein, which can connect the acoustic properties of protein nanostructures with calcium concentration. The disclosed compositions and methods can provide simultaneous access to large volume in deep tissue. The methods and compositions provided herein find use in a number areas, including neuroscience, immunology, organismal development, endocrinology, and cardiology. Provided herein include compositions, methods, and systems for calcium imaging which, in some embodiments, do not require sophisticated equipment, do not require high concentrations of calcium indicators to be administered, do not require subjects to be anesthetized, are not difficult to encode genetically, do not involve acute intracranial injections, and do not suffer from the strong background of hemoglobin-based signals.

Disclosed herein include compositions (e.g., nucleic acid composition, a Ca²⁺-sensing GvpC protein, Ca²⁺-sensing gas vesicles). There are provided, in some embodiments, nucleic acid compositions. The nucleic acid composition can comprise: one or more promoters operably connected to one or more gas vesicle (GV) polynucleotides comprising: a Ca²⁺-sensing GvpC gene encoding a Ca²⁺-sensing GvpC protein. In some embodiments, the Ca²⁺-sensing GvpC protein is capable of associating with a gas vesicle structural (GVS) protein to form a Ca²⁺-sensing gas vesicle (GV).

There are provided, in some embodiments, nucleic acid compositions. The nucleic acid composition can comprise: one or more promoters operably connected to one or more gas vesicle (GV) polynucleotides comprising: one or more gas vesicle assembly (GVA) gene(s) encoding one or more GVA protein(s), one or more gas vesicle structural (GVS) gene(s) encoding one or more GVS protein(s) selected from GvpA and GvpB, and/or a Ca²⁺-sensing GvpC gene encoding a Ca²⁺-sensing GvpC protein. In some embodiments, the one or more GVA protein(s), the one or more GVS protein(s), and the Ca²⁺-sensing GvpC protein are capable of forming Ca²⁺-sensing gas vesicles (GVs) upon expression in a cell or a cell-like environment.

There are provided, in some embodiments, compositions. In some embodiments, the composition comprises: a Ca²⁺-sensing GvpC protein, wherein the Ca²⁺-sensing GvpC protein is capable of associating with a gas vesicle structural (GVS) protein to form a Ca²⁺-sensing GV.

There are provided, in some embodiments, compositions. In some embodiments, the composition comprises: a Ca²⁺-sensing gas vesicle (GV) comprising a Ca²⁺-sensing GvpC protein and one or more GVS protein(s) selected from GvpA and GvpB.

In some embodiments, the Ca²⁺-sensing GV comprises a gas enclosed by a protein shell comprising the Ca²⁺-sensing GvpC protein and a GVS protein selected from GvpA and GvpB. The Ca²⁺-sensing GvpC protein can comprise a Ca²⁺-binding domain. The Ca²⁺-binding domain can be capable of binding 1, 2, 3, 4, 5, or 6 Ca²⁺ ions. The Ca²⁺-sensing GvpC protein can be capable of undergoing a first allosteric conformational change upon the Ca²⁺-binding domain binding Ca²⁺ that causes the Ca²⁺-sensing GV to change from a GV stiff state to a GV soft state. The Ca²⁺-binding domain binding Ca²⁺ can comprise the Ca²⁺-binding domain binding 1, 2, 3, 4, 5, or 6 Ca²⁺ ions. The Ca²⁺-sensing GvpC protein can be capable of undergoing a second allosteric conformational change upon the Ca²⁺-binding domain releasing bound Ca²⁺ that causes the Ca²⁺-sensing GV to change from a GV soft state to a GV stiff state. The Ca²⁺-binding domain releasing bound Ca²⁺ can comprise the Ca²⁺-binding domain releasing 1, 2, 3, 4, 5, or 6 bound Ca²⁺ ions. In some embodiments, one or more of the mechanical, acoustic, surface, and magnetic properties of the Ca²⁺-sensing GV differ between the GV soft state to the GV stiff state. The acoustic contrast of the Ca²⁺-sensing GV can be capable of reversibly changing in response to local Ca²⁺ concentrations. In some embodiments, a Ca²⁺-sensing GV is capable of: (i) binding Ca²⁺, optionally 1, 2, 3, 4, 5, or 6 Ca²⁺ ions; and (ii) releasing bound Ca²⁺, optionally 1, 2, 3, 4, 5, or 6 bound Ca²⁺ ions, recurrently in response to changes in Ca²⁺ dynamics.

In some embodiments, the Ca²⁺-sensing GvpC protein comprises an interaction domain configured to: bind the Ca²⁺-binding domain upon the Ca²⁺-binding domain binding Ca²⁺, optionally upon the Ca²⁺-binding domain binding 1, 2, 3, 4, 5, or 6 Ca²⁺ ions, optionally the first allosteric conformational change comprises the interaction domain binding the Ca²⁺-binding domain; and detach from the Ca²⁺-binding domain upon the Ca²⁺-binding domain releasing bound Ca²⁺, optionally upon the Ca²⁺-binding domain releasing 1, 2, 3, 4, 5, or 6 bound Ca²⁺ ions, optionally the second allosteric conformational change comprises the interaction domain detaching from the Ca²⁺-binding domain. In some embodiments, the Ca²⁺-sensing GvpC protein comprises an interaction domain configured to: detach from the Ca²⁺-binding domain upon the Ca²⁺-binding domain binding Ca²⁺, optionally upon the Ca²⁺-binding domain binding 1, 2, 3, 4, 5, or 6 Ca²⁺ ions, optionally the second allosteric conformational change comprises the interaction domain detaching from the Ca²⁺-binding domain; and bind the Ca²⁺-binding domain upon the Ca²⁺-binding domain releasing bound Ca²⁺, optionally upon the Ca²⁺-binding domain releasing 1, 2, 3, 4, 5, or 6 bound Ca²⁺ ions, optionally the first allosteric conformational change comprises the interaction domain binding the Ca²⁺-binding domain.

In some embodiments, the Ca²⁺-sensing GvpC protein comprises: an N-terminal domain; a C-terminal domain; and a central domain situated between N-terminal domain and the C-terminal domain, wherein the central domain comprises two or more tandem repeat regions. In some embodiments, each repeat region can be an alpha-helical polypeptide. The two or more tandem repeat regions can be 2, 3, 4, 5, or 6, tandem repeat regions; and/or share an amino acid sequence identity of at least about 50%. The Ca²⁺-binding domain can be N-terminal of the N-terminal domain. The Ca²⁺-binding domain can be C-terminal of the C-terminal domain. The Ca²⁺-binding domain can be connected to the N-terminal domain or the C-terminal domain via a first linker. The interaction domain can be situated within the central domain. The interaction domain can be situated within one of the two or more repeat regions.

The interaction domain can be flanked by a second linker and a third linker, the second linker can be N-terminal of the interaction domain and the third linker can be C-terminal of the interaction domain. In some embodiments, the first linker, second linker, and/or third linker: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; comprises 3 repeating amino acid subunits or more; and/or comprises an amino acid sequence selected from the group comprising GSGSGSG (SEQ ID NO: 1), GGGGS (SEQ ID NO: 2), GSGSG (SEQ ID NO: 3), GGGG (SEQ ID NO: 4), GGG (SEQ ID NO: 5), GG (SEQ ID NO 6), GS (SEQ ID NO: 7), GSGS (SEQ ID NO: 8), GGGS (SEQ ID NO: 9), GGS (SEQ ID NO: 10), GTS (SEQ ID NO: 11), GGSGGS (SEQ ID NO: 12), GGG (SEQ ID NO: 13), GGGGGG (SEQ ID NO: 14), GGGGGGGGG (SEQ ID NO: 15), GGGGGGGGGGGG (SEQ ID NO: 16), GGGGGGGGGGGGGGG (SEQ ID NO: 17), GGS (SEQ ID NO: 18), GGSGGS (SEQ ID NO: 19), GGSGGSGGS (SEQ ID NO: 20), GGSGGSGGSGGS (SEQ ID NO: 21), GGSGGSGGSGGSGGS (SEQ ID NO: 22), GSG (SEQ ID NO: 23), GSGGSG (SEQ ID NO: 24), GSGGSGGSG (SEQ ID NO: 25), GSGGSGGSGGSG (SEQ ID NO: 26), GSGGSGGSGGSGGSG (SEQ ID NO: 27), (GGGGS), (SEQ ID NO: 28), GGGGSGGGGS (SEQ ID NO: 29), GGGGSGGGGSGGGGS (SEQ ID NO: 30), GGGGSGGGGSGGGGSGGGGS (SEQ ID NO: 31), and GGGGSGGGGSGGGGSGGGGSGGGGS (SEQ ID NO: 32).

The Ca²⁺-sensing GvpC protein can be capable of sensing Ca²⁺ dynamics at sub-micromolar concentrations with a sub-minute time scale. In some embodiments, the Ca²⁺-binding domain binding detectably binds Ca²⁺ with a dissociation constant (K_{d}) of less than about 1000 nM (e.g., 1000 nM, 900 nM, 800 nM, 700 nM, 600 nM, 500 nM, 400 nM, 300 nM, 200 nM, 100 nM, 50 nM, 25 nM, 10 nM, 1 nM, or a number or a range between any of these values). In some embodiments, the Ca²⁺-sensing GV produces reversible non-linear ultrasound contrast in response to Ca²⁺ with half-maximal contrast occurring at less than about 1000 nM (e.g., 1000 nM, 900 nM, 800 nM, 700 nM, 600 nM, 500 nM, 400 nM, 300 nM, 200 nM, 100 nM, 50 nM, 25 nM, 10 nM, 1 nM, or a number or a range between any of these values). In some embodiments, the Ca²⁺-sensing GV exhibits a detectable half-rise time of less than about 60 seconds (e.g., 60 s, 50 s, 40 s, 30 s, 20 s, 10 s, 5 s, 1 s, 0.1 s, 0.01 s, or a number or a range between any of these values) upon contact with Ca²⁺. In some embodiments, the Ca²⁺-sensing GV exhibits a detectable half-decay time of less than about 60 seconds (e.g., 60 s, 50 s, 40 s, 30 s, 20 s, 10 s, 5 s, 1 s, 0.1 s, 0.01 s, or a number or a range between any of these values) upon removal of Ca²⁺. The Ca²⁺ affinity and/or response kinetics of the Ca²⁺-sensing GV can be capable of being tuned by configuring one or more of the Ca²⁺-binding domain, the interaction domain, the first linker, the second linker, the third linker, and the insertion positions of the Ca²⁺-binding domain and the interaction domain within the parental GvpC protein.

The Ca²⁺-sensing GvpC protein can be derived from Anabaena flos-aquae (SEQ ID NO: 33), Halobacterium salinarum (SEQ ID NO: 34), Halobacterium mediterranei (SEQ ID NO: 35), Microchaete diplosiphon (SEQ ID NO: 36), Nostoc sp. (SEQ ID NO: 37), or a combination thereof. The Ca²⁺-sensing GvpC protein can comprise one or more truncation(s), insertion(s), and mutation(s) as compared to the parental GvpC protein from which it is derived. The Ca²⁺-sensing GvpC protein can comprise fewer tandem repeat regions than the parental GvpC protein. The Ca²⁺-sensing GvpC protein can comprise an amino acid sequence that is at least 80%, 85%, 90%, 95%, 98%, 99%, or 100% identical to any one of SEQ ID NOS: 38 and 43-45, or a portion thereof. The nucleic acid composition can comprise a DNA sequence that is at least 80%, 85%, 90%, 95%, 98%, 99%, or 100% identical to any one of SEQ ID NOS: 39-42, or a portion thereof. The interaction domain can comprise an amino acid sequence that is at least 80%, 85%, 90%, 95%, 98%, 99%, or 100% identical to any one of SEQ ID NOs: 46-51, or a portion thereof. The Ca²⁺-binding domain can comprise an amino acid sequence that is at least 80%, 85%, 90%, 95%, 98%, 99%, or 100% identical to any one of SEQ ID NOs: 52-58, or a portion thereof. The Ca²⁺-binding domain can comprise calmodulin (CaM) or a derivative thereof, optionally a mutant human CaM. The interaction domain can comprise or can be derived from CaMKI. The Ca²⁺-sensing GvpC protein can comprise 3 tandem repeat regions, wherein the interaction domain is inserted within the second of the 3 tandem repeat regions. The Ca²⁺-sensing GvpC protein can be derived from a parental five tandem repeat GvpC protein natively expressed in Anabaena flos-aquae. In some embodiments, one or more of the mechanical, acoustic, surface and/or magnetic properties of the Ca²⁺-sensing GV can be capable of being tuned by configuring one or more of the Ca²⁺-binding domain, the interaction domain, the first linker, the second linker, the third linker, and the insertion positions of the Ca²⁺-binding domain and the interaction domain within the parental GvpC protein.

TABLE 1
AMINO ACID SEQUENCES

| | SEQ | |
|---|---|---|
| | ID | |
| Name | NO | Sequence |
| UGECI-281 | 38 | MISLMAKIRQEHQSIAEKVAELSLETREFLSVTTAKRQEQAEKQAQELQ |
| GvpC | | AFYKDLQETSKSKWKQAFNATAVVRHMRKQELLAFHKELQETSQQFLSA |
| | | TAQARIAQAEKQAQELLAFRQDLFVSIFGGGGGSGGGGSGGGGSGGGGS |
| | | GGGGSGGGGSGGGGSGGGGSDQLTEEQIAEFKEEFSLFAKDGDGTITTK |
| | | ELGTVMRSLGQNPTEAELQDMINEVDADGDGTIDFPEFLTMMARKMKYR |
| | | DTEEEIREAFGVFDKDGNGYISAAELRHVMTNLGEKLTDEEVDEMIREA |
| | | DIDGDGQVNYEEFVQMMTAKSLEHHHHHH* |
| UGECI-141 | 43 | MISLMAKIRQEHQSIAEKVAELSLETREFLSVTTAKRQEQAEKQAQELQA |
| GvpC | | FYKDLQETSRWKKNFIAVSAANRFKKRKQELLAFHKELQETSQQFLSATA |
| | | QARIAQAEKQAQELLAFRQDLFVSIFGGGGGSGGGGSGGGGSGGGGSDQL |
| | | TEEQIAEFKEEFSLFDKDGDGTITTKELGTVMRSLGQNPTEAELQDMINE |
| | | VDADGDGTIDFPEFLTMMARKMKYRDTEEEIREAFGVFAKDGNGYISAAE |
| | | LRHVMTNLGEKLTDEEVDEMIREADIDGDGQVNYEEFVQMMTAKSLEHHH |
| | | HHH* |
| UGECI-241 | 44 | MISLMAKIRQEHQSIAEKVAELSLETREFLSVTTAKRQEQAEKQAQELQA |
| GvpC | | FYKDLQETSKSKWKQAFNATAVVRHMRKQELLAFHKELQETSQQFLSATA |
| | | QARIAQAEKQAQELLAFRQDLFVSIFGGGGGSGGGGSGGGGSGGGGSDQL |
| | | TEEQIAEFKEEFSLFAKDGDGTITTKELGTVMRSLGQNPTEAELQDMINE |
| | | VDADGDGTIDFPEFLTMMARKMKYRDTEEEIREAFGVFDKDGNGYISAAE |
| | | LRHVMTNLGEKLTDEEVDEMIREADIDGDGQVNYEEFVQMMTAKSLEHHH |
| | | HHH* |
| UGECI-381RK | 45 | MISLMAKIRQEHQSIAEKVAELSLETREFLSVTTAKRQEQAEKQAQELQA |
| GvpC | | FYKDLQETSRWKKAFIAVSAANRFKKRKQELLAFHKELQETSQQFLSATA |
| | | QARIAQAEKQAQELLAFRQDLFVSIFGGGGGSGGGGSGGGGSGGGGSGGG |
| | | GSGGGGSGGGGSGGGGSDQLTEEQIAEFKEEFSLFAKDGDGTITTKELGT |
| | | VMRSLGQNPTEAELQDMINEVDADGDGTIDFPEFLTMMARKMKYRDTEEE |
| | | IREAFGVFDKDGNGYISAAELRHVMTNLGEKLTDEEVDEMIREADIDGDG |
| | | QVNYEEFVQMMTAKTSGSHHHHHH* |
| RS20-smMLCK | 46 | RRKLKAAVKAVVASSRLGS |
| M13-skMLCK* | 47 | KRRWKKNFIAVSAANRFKKISSSGAL |
| CaMKI* | 48 | MLGAVEGPRWKQAEDIRDIYDFRDVLGTGAFSEVILAEDKRTQKLVAIKC |
| | | IAKEALEGKEGSMENEIAVLHKIKHPNIVALDDIYESGGHLYLIMQLVSG |
| | | GELFDRIVEKGFYTERDASRLIFQVLDAVKYLHDLGIVHRDLKPENLLYY |
| | | SLDEDSKIMISDFGLSKMEDPGSVLSTACGTPGYVAPEVLAQKPYSKAVD |
| | | CWSIGVIAYILLCGYPPFYDENDAKLFEQILKAEYEFDSPYWDDISDSAK |
| | | DFIRHLMEKDPEKRFTCEQALQHPWIAGDTALDKNIHQSVSEQIKKNFAK |
| | | SKWKQAFNATAVVRHMRKLQLGTSQEGQGQTASHGELLTPVAGGPAAGCC |
| | | CRDCCVEPGTELSPTLPHQL |
| CBP1 (modified | 49 | RWKKNFIAVSAANRFKKRK |
| from M13- | | |
| skMLCK) | | |
| CBP2 (part of | 50 | KSKWKQAFNATAVVRHMRK |
| CaMKI) | | |
| CBP3 (modified | 51 | RWKKAFIAVSAANRFKKRK |
| from M13- | | |
| skMLCK) | | |
| CaM-WT | 52 | DQLTEEQIAEFKEAFSLFDKDGDGTITTKELGTVMRSLGQNPTEAELQDM |
| (human | | INEVDADGNGTIDFPEFLTMMARKMKDTDSEEEIREAFRVFDKDGNGYIS |
| calmodulin) | | AAELRHVMTNLGEKLTDEEVDEMIREADIDGDGQVNYEEFVQMMTAK |
| CaM5K | 53 | DQLTEEQIAEFKEAFSLFDKDGDGTITTKELGTVMRSLGQNPTEAELQDM |
| (calmodulin part | | INEVDADGDGTIDFPEFLTMMARKMKYTDSEEEIREAFGVFDKDGNGYIS |
| in GCaMP5k) | | AAELRHVMTNLGEKLTDEEVDEMIREADIDGDGQVNYEEFVQMMTAK |
| CaM6f | 54 | DQLTEEQIAEFKEEFSLFDKDGDGTITTKELGTVMRSLGQNPTEAELQDM |
| (calmodulin part | | INEVDADGDGTIDFPEFLTMMARKMKYRDTEEEIREAFGVFDKDGNGYIS |
| in GCaMP6f) | | AAELRHVMTNLGEKLTDEEVDEMIREADIDGDGQVNYEEFVQMMTAK |
| CaM6f-EFl | 55 | DQLTEEQIAEFKEEFSLFAKDGDGTITTKELGTVMRSLGQNPTEAELQDM |
| (mutated CaM6f) | | INEVDADGDGTIDFPEFLTMMARKMKYRDTEEEIREAFGVFDKDGNGYIS |
| | | AAELRHVMTNLGEKLTDEEVDEMIREADIDGDGQVNYEEFVQMMTAK |
| CaM6f-EF2 | 56 | DQLTEEQIAEFKEEFSLFDKDGDGTITTKELGTVMRSLGQNPTEAELQDM |
| (mutated CaM6f) | | INEVAADGDGTIDFPEFLTMMARKMKYRDTEEEIREAFGVFDKDGNGYIS |
| | | AAELRHVMTNLGEKLTDEEVDEMIREADIDGDGQVNYEEFVQMMTAK |
| CaM6f-EF3 | 57 | DQLTEEQIAEFKEEFSLFDKDGDGTITTKELGTVMRSLGQNPTEAELQDM |
| (mutated CaM6f) | | INEVDADGDGTIDFPEFLTMMARKMKYRDTEEEIREAFGVFAKDGNGYIS |
| | | AAELRHVMTNLGEKLTDEEVDEMIREADIDGDGQVNYEEFVQMMTAK |
| CaM6f-EF4 | 58 | DQLTEEQIAEFKEEFSLFDKDGDGTITTKELGTVMRSLGQNPTEAELQDM |
| (mutated CaM6f) | | INEVDADGDGTIDFPEFLTMMARKMKYRDTEEEIREAFGVFDKDGNGYIS |
| | | AAELRHVMTNLGEKLTDEEVDEMIREAAIDGDGQVNYEEFVOMMTAK |

*Bold indicates portion used in the UGECI sequence

In some embodiments, the first allosteric conformational change causes an at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) reduction in the mechanical stiffness of Ca²⁺-sensing GV. The fold reduction in mechanical stiffness can be capable of being tuned by configuring one or more of the Ca²⁺-binding domain, the interaction domain, the first linker, the second linker, the third linker, and the insertion positions of the Ca²⁺-binding domain and the interaction domain within the parental GvpC protein. The mechanical stiffness of a Ca²⁺-sensing GV in a GV stiff state can be least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) greater than the mechanical stiffness of Ca²⁺-sensing GV in a GV soft state, and a Ca²⁺-sensing GV in a GV soft state can be capable of producing at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) greater non-linear ultrasound signals as compared to a Ca²⁺-sensing GV in a GV stiff state. A Ca²⁺-sensing GV in a GV soft state can be capable of exhibiting an about 5 dB to about 50 dB (e.g., 5 dB, 10 dB, 15 dB, 25 dB, 30 dB, 35 dB, 40 dB, 50 dB, or a number or a range between any of these values) enhancement in nonlinear ultrasound contrast as compared to a Ca²⁺-sensing GV in a GV stiff state. The dynamic range of a Ca²⁺-sensing GV in a mammalian cell can be about 5 dB to about 50 dB (e.g., 5 dB, 10 dB, 15 dB, 25 dB, 30 dB, 35 dB, 40 dB, 50 dB, or a number or a range between any of these values), optionally about 10-12 dB. A Ca²⁺-sensing GV in a GV soft state can be capable of exhibiting an at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) increase in contrast to noise ratio (CNR) as compared to a Ca²⁺-sensing GV in a GV stiff state.

The systems, methods, compositions, and kits provided herein can, in some embodiments, be employed in concert with the systems, methods, compositions, and kits comprising recombinant adeno-associated virus (rAAV) comprising an AAV acoustic targeting peptide exhibiting increased transduction at site(s) of focused ultrasound blood-brain barrier opening (FUS-BBBO), increased neuronal tropism, and diminished transduction of peripheral organs described in U.S. patent application Ser. No. 17/814,384, entitled, “VIRAL VECTORS FOR ENHANCED ULTRASOUND-MEDIATED DELIVERY TO THE BRAIN,” filed Jul. 22, 2022, the content of which is incorporated herein by reference in its entirety.

GV production requires the co-expression of multiple GV proteins (Gyps), which in prokaryotes are expressed from polycistronic operons at specific ratios determined by the strength of their respective ribosome binding sites or other regulatory mechanisms. The systems, methods, compositions, and kits provided herein can, in some embodiments, be employed in concert with the systems, methods, compositions, and kits expression of multiple proteins from a single mRNA with a predetermined stoichiometry described in U.S. patent application Ser. No. 17/866,240, entitled “STOICHIOMETRIC EXPRESSION OF MESSENGER POLYCISTRONS”, filed Jul. 15, 2022, the content of which is incorporated herein by reference in its entirety. The systems, methods, compositions, and kits provided herein can, in some embodiments, be employed in concert with the systems, methods, compositions, and kits described in U.S. patent application Ser. No. 17/936,286, entitled, “VIRAL DELIVERY OF GAS VESICLE GENES,” filed Sep. 28, 2022, the content of which is incorporated herein by reference in its entirety.

Engineered Ca²⁺-sensing Gas Vesicles

The GVA gene(s) and/or GVS gene(s) can be derived from a species of Anabaena bacteria, Halobacterium salinarum, and/or Bacillus megaterium. In some embodiments, the one or more GV polynucleotides comprises: two or more GVS genes derived from different prokaryotic species; GVA genes and/or GVS genes from Bacillus Megaterium, Anabaena flos-aquae, Serratia sp., Burkholderia thailandensis, B. megaterium, Frankia sp, Haloferax mediaterranei, Halobacterium sp, Microchaete diplosiphon, Nostoc sp, Halorubrum vacuolatum, Microcystis aeruginosa, Methanosarcina barkeri, Streptomyces coelicolor, and/or Psychromonas ingrahamii; gvpB, gvpN gvpF, gvpG, gvpL gvpS, gvpK, gvpJ, and/or gvpU from B. megaterium; gvpA, gvpN, gvpJ, gvpK, gvpF, gvpG, gvpV, and/or gvpW from Anabaena flos-aquae; gvpR, gvpN, gvpF, gvpG, gvpL, gvpS, gvpK, gvpJ, gvpT and/or gvpU from B. megaterium and gvpA from Anabaena flos-aquae; gvpA from Anabaena flos-aquae, and gvpN, gvpF, gvpG, gvpL, gvpS, gvpK, gvpJ, and/or gvpU from B. megaterium; and/or gvpA and/or gvpN from Anabaena flos-aquae, and gvpF, gvpG, gvpL, gvpS, gvpK, gvpJ, and/or gvpU from B. megaterium. In some embodiments, the GVA genes and/or GVS genes have sequences codon optimized for expression in a eukaryotic cell. The Ca²⁺-sensing GV can be a hybrid GV derived from two or more prokaryotic species.

The Ca²⁺-sensing GV in a GV soft state can have a first buckling pressure profile. The first buckling pressure profile can comprise a buckling function from which a Ca²⁺-sensing GV in a GV soft state buckling amount can be determined for a given pressure value. The buckling amount can comprise the amount of nonlinear contrast. The first buckling pressure profile can comprise a first buckling threshold pressure where a Ca²⁺-sensing GV in a GV soft state starts to buckle and produce nonlinear contrast, a first optimum buckling pressure where a Ca²⁺-sensing GV in a GV soft state exhibits maximum buckling and produces the highest level of nonlinear contrast, a first collapse pressure wherein a Ca²⁺-sensing GV in a GV soft state collapses, any pressure between the first buckling threshold pressure and the first optimum buckling pressure, and any pressure between the first optimum buckling pressure and the first collapse pressure. The Ca²⁺-sensing GV in a GV stiff state can have a second buckling pressure profile. The second buckling pressure profile can comprise a buckling function from which a Ca²⁺-sensing GV in a GV stiff state buckling amount can be determined for a given pressure value. The buckling amount can comprise the amount of nonlinear contrast. The second buckling pressure profile can comprise a second buckling threshold pressure where a Ca²⁺-sensing GV in a GV stiff state starts to buckle and produce nonlinear contrast, a second optimum buckling pressure where a Ca²⁺-sensing GV in a GV stiff state exhibits maximum buckling and produces the highest level of nonlinear contrast, a second collapse pressure wherein a Ca²⁺-sensing GV in a GV stiff state collapses, any pressure between the second buckling threshold pressure and the second optimum buckling pressure, and any pressure between the second optimum buckling pressure and the second collapse pressure. The first buckling pressure profile and the second buckling pressure profile can be different. The first buckling pressure profile and/or the second buckling pressure profile can be capable of being tuned by configuring one or more of the Ca²⁺-binding domain, the interaction domain, the first linker, the second linker, the third linker, and the insertion positions of the Ca²⁺-binding domain and the interaction domain within the parental GvpC protein. A selectable buckling pressure can be the pressure value which produces the maximal difference in buckling between a Ca²⁺-sensing GV in a GV soft state and a Ca²⁺-sensing GV in a GV stiff state. In some embodiments, the selectable buckling pressure is: from about 40 kPa to about 1500 kPa; any collapse pressure within the first buckling pressure profile; any collapse pressure within the second buckling pressure profile; the first optimum buckling pressure; and/or the second optimum buckling pressure.

The Ca²⁺-sensing GV in a GV soft state can have a first collapse pressure profile. The first collapse pressure profile can comprise a collapse function from which a Ca²⁺-sensing GV in a GV soft state collapse amount can be determined for a given pressure value. The first collapse pressure profile can comprise a first initial collapse pressure where 5% or lower of a plurality of Ca²⁺-sensing GVs in a GV soft state are collapsed, a first midpoint collapse pressure where 50% of a plurality of Ca²⁺-sensing GVs in a GV soft state are collapsed, a first complete collapse pressure where at least 95% of a plurality of Ca²⁺-sensing GVs in a GV soft state are collapsed, any pressure between the first initial collapse pressure and the first midpoint collapse pressure, and any pressure between the first midpoint collapse pressure and the first complete collapse pressure. In some embodiments, a first selectable collapse pressure is: any collapse pressure within the first collapse pressure profile; selected from the first collapse pressure profile at a value between 0.05% collapse of a plurality of Ca²⁺-sensing GVs in a GV soft state and 95% collapse of a plurality of Ca²⁺-sensing GVs in a GV soft state; equal to or greater than the first initial collapse pressure; equal to or greater than the first midpoint collapse pressure; and/or equal to or greater than the first complete collapse pressure. The Ca²⁺-sensing GV in a GV stiff state can have a second collapse pressure profile. The second collapse pressure profile can comprise a collapse function from which a Ca²⁺-sensing GV in a GV stiff state collapse amount can be determined for a given pressure value. The first collapse pressure profile and the second collapse pressure profile can be different. The first collapse pressure profile and/or second collapse pressure profile can be capable of being tuned by configuring one or more of the Ca²⁺-binding domain, the interaction domain, the first linker, the second linker, the third linker, and the insertion positions of the Ca²⁺-binding domain and the interaction domain within the parental GvpC protein. A midpoint of the second collapse profile can have a higher pressure component than a midpoint of the first collapse profile. The second collapse pressure profile can comprise a second initial collapse pressure where 5% or lower of a plurality of Ca²⁺-sensing GVs in a GV stiff state are collapsed, a second midpoint collapse pressure where 50% of a plurality of Ca²⁺-sensing GVs in a GV stiff state are collapsed, a second complete collapse pressure where at least 95% of a plurality of Ca²⁺-sensing GVs in a GV stiff state are collapsed, any pressure between the second initial collapse pressure and the second midpoint collapse pressure, and any pressure between the second midpoint collapse pressure and the second complete collapse pressure. In some embodiments, a second selectable collapse pressure is: any collapse pressure within the second collapse pressure profile; selected from the second collapse pressure profile at a value between 0.05% collapse of a plurality of Ca²⁺-sensing GVs in a GV stiff state and 95% collapse of a plurality of Ca²⁺-sensing GVs in a GV stiff state; equal to or greater than the second initial collapse pressure; equal to or greater than the second midpoint collapse pressure; and/or equal to or greater than the second complete collapse pressure.

In some embodiments, the one or more GV polynucleotide(s) comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element (e.g., woodchuck hepatitis post-translational regulatory element (WPRE), bovine growth hormone polyadenylation (bGH-polyA) signal sequence, human growth hormone polyadenylation (hGH-polyA) signal sequence, or any combination thereof). In some embodiments, one or more GV polynucleotides are operably connected to a promoter selected from the group comprising: a minimal promoter (e.g., TATA, miniCMV, and/or miniPromo); a ubiquitous promoter; a tissue-specific promoter and/or a lineage-specific promoter; and/or a ubiquitous promoter (e.g., cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin ((3-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

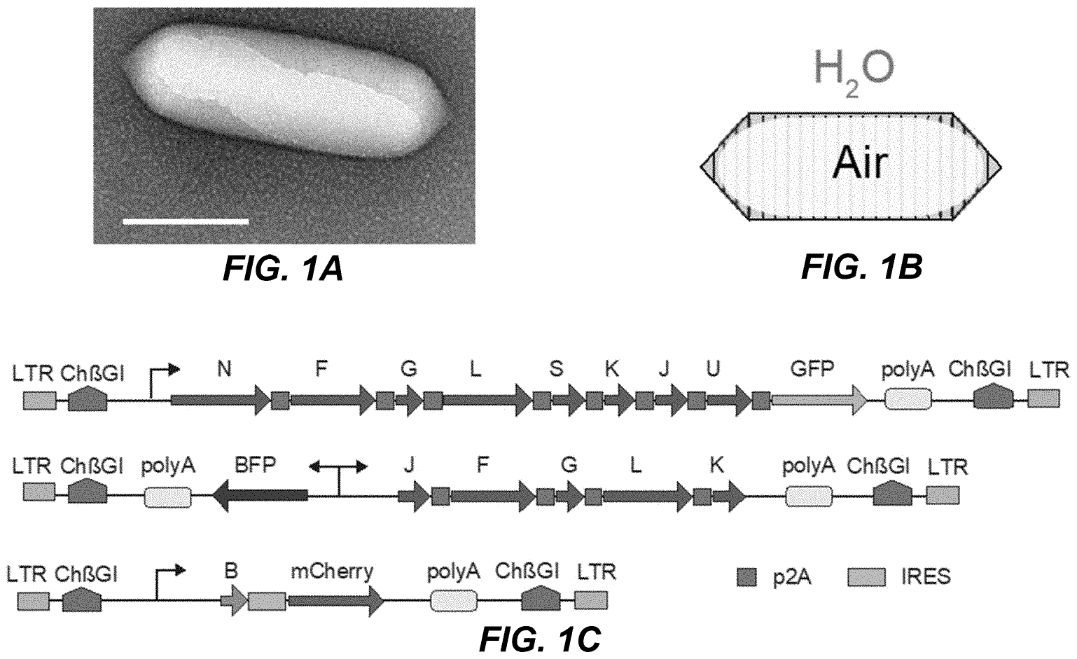

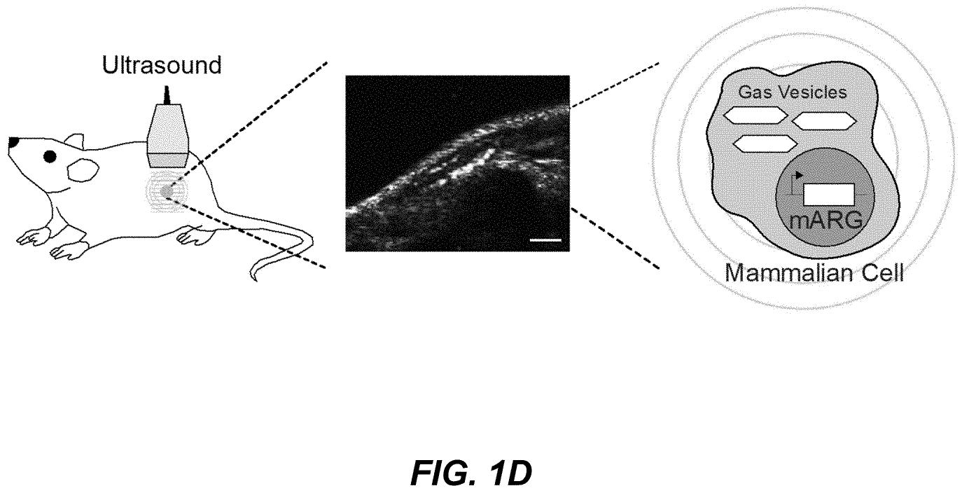

In some embodiments, nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The nucleic acid composition can comprise a plurality of vectors (e.g., viral vectors). For example, the compositions provided herein include a first viral vector encoding one or more GVS proteins and a second viral vector encoding one or more GVA proteins (see, e.g., FIG. 1C).

Provided herein are engineered gas-filled protein structures (GVPS), also referred to as “gas vesicles” (GVs). The phrases “gas vesicles protein structure” or “GV”, “GVP”, “GVPS” or “Gas Vesicles” as used herein shall be given their ordinary meaning, and shall also refer to a gas-filled protein structure intracellularly expressed by certain bacteria or archea as a mechanism to regulate cellular buoyancy in aqueous environments. GVs are described in Walsby, A. E. ((1994). Gas vesicles. Microbiology and Molecular Biology Reviews, 58(1), 94-144) hereby incorporated by reference in its entirety. The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments. In particular, GVS shell comprises a GVS identified as gvpA or gvpB (herein also referred to as gvpA/B) and optionally also a GVS identified as gvpC. The compositions, methods and systems described herein can be used with compositions, methods and systems (e.g., gas vesicle compositions and ultrasonic methods) previously described in U.S. Patent Application Publication Nos. 2014/0288411, 2014/0288421, 2018/0030501, 2018/0038922, 2019/0175763, 2019/0314001, 2020/0164095, 2020/0237346, 2021/0060185, and International Patent Application Publication WO2020/146379; the content of each of these applications is incorporated herein by reference in its entirety.

In particular, a GV in the sense of the disclosure is a structure intracellularly expressed by bacteria or archaea forming a hollow structure wherein a gas is enclosed by a protein shell, which is a shell substantially made of protein (up at least 95% protein). In gas vesicles in the sense of the disclosure, the protein shell is formed by a plurality of proteins herein also indicated as Gyp proteins or Gvps, which are expressed by the bacteria or archaea and form in the bacteria or archaea cytoplasm a gas permeable and liquid impermeable protein shell configuration encircling gas. Accordingly, a protein shell of a GV is permeable to gas but not to surrounding liquid such as water. For example, GVs' protein shells exclude liquid water but permit gas to freely diffuse in and out from the surrounding media making them physically stable despite their usual nanometer size.

GV structures are typically nanostructures with widths and lengths of nanometer dimensions (in particular with widths of 45-250 nm and lengths of 100-800 nm) but can have lengths as large as the dimensions of a cell in which they are expressed, as will be understood by a skilled person. GVs and methods are described in Farhadi et al, Science, 2019, hereby incorporated by reference. In certain embodiments, the gas vesicles protein structure have average dimensions of 1000 nm or less, such as 900 nm or less, including 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 75 nm or less, or 50 nm or less. For example, the average diameter of the gas vesicles may range from 10 nm to 1000 nm, such as 25 nm to 500 nm, including 50 nm to 250 nm, or 100 nm to 250 nm. By “average” is meant the arithmetic mean.

GVs in the sense of the disclosure have different shapes depending on their genetic origins. For example, GVs in the sense of the disclosure can be substantially spherical, ellipsoid, cylindrical, or have other shapes such as football shape or cylindrical with cone shaped end portions depending on the type of bacteria providing the gas vesicles.

The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments. In particular, GVS shell comprises a GVS identified as gvpA or gvpB (herein also referred to as Gyp AB) and optionally also a GVS identified as gvpC. GvpA is a structural protein that assembles through repeated unites to make up the bulk of GVs. GvpC is a scaffold protein with 5 repeat units that assemble on the outer shell of GVs. GvpC can be engineered to tune the mechanical and acoustic properties of GVs as well as act as a handle for appending moieties on to. A gvpC protein is a hydrophilic protein of a GV shell, which includes repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another.

The optional gvpC gene encodes for a gvpC protein which is a hydrophilic protein of a GV shell, including repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another. In performing alignment steps sequence are identified as repeat when the sequence shows at least 3 or more of the characteristics described in U.S. application Ser. No. 15/663,635 published as US 2018/0030501 (incorporated herein by reference in its entirety) which also include additional features of gvpC proteins and the related identification.

The phrase “GV type” as used herein shall be given its ordinary meaning, and shall also refer to a gas vesicle having dimensions and shape resulting in distinctive mechanical, acoustic, surface and/or magnetic properties as will be understood by a skilled person upon reading of the present disclosure. In particular, a skilled person will understand that different shapes and dimensions will result in different properties in view of the indications in provided in U.S. application Ser. No. 15/613,104 and U.S. Ser. No. 15/663,600 and additional indications identifiable by a skilled person. In some embodiments, the nucleic acid compositions provided herein encode a combination of different GV types and/or variants thereof, with each expressed GV exhibiting a different acoustic collapse profile with progressively decreased midpoint collapse pressure values. In some embodiments, the percentage difference between the midpoint collapse pressure values of any given two expressed GVs types is at least twenty percent.

In some embodiments, GVs are capable of withstanding pressures of several kPa. but collapse irreversibly at a pressure at which the GV protein shell is deformed to the point where it flattens or breaks, allowing the gas inside the GV to dissolve irreversibly in surrounding media, herein also referred to as a critical collapse pressure, or selectable critical collapse pressure, as there are various points along a collapse pressure profile (e.g., peak acoustic pressure).

A collapse pressure profile (e.g., peak acoustic pressure) as used herein indicates a range of pressures over which collapse of a population of GVs of a certain type occurs. In particular, a collapse pressure profile in the sense of the disclosure comprise increasing acoustic collapse pressure values, starting from an initial collapse pressure value at which the GV signal/optical scattering by GVs starts to be erased to a complete collapse pressure value at which the GV signal/optical scattering by GVs is completely erased. The collapse pressure profile of a set type of GV is thus characterized by a mid-point pressure where 50% of the GVs of the set type have been collapsed (also known as the “midpoint collapse pressure”), an initial collapse pressure where 5% or lower of the GVs of the type have been collapsed, and a complete collapse pressure where at least 95% of the GVs of the type have been collapsed. In some embodiments herein described a selectable critical collapse pressure can be any of these collapse pressures within a collapse pressure profile, as well as any point between them. The critical collapse pressure profile of a GV is functional to the mechanical properties of the protein shell and the diameter of the shell structure. U.S. Patent Application Publication No. 2020/0164095 describes gas vesicles, protein variants and related compositions methods and systems for singleplexed and/or multiplexed ultrasonic methods (e.g., imaging of a target site in which a gas vesicle provides contrast for the imaging) which is modifiable by application of a selectable acoustic collapse pressure value of the gas vesicle, the content of which is hereby expressly incorporated by reference in its entirety.

The acoustic collapse pressure profile (e.g., peak acoustic pressure) of a given GV type can be determined by imaging GVs with imaging ultrasound energy after collapsing portions of the given GV type population with a collapsing ultrasound energy (e.g. ultrasound pulses) with increasing peak positive pressure amplitudes to obtain acoustic pressure data point of acoustic pressure values, the data points forming an acoustic collapse curve. The acoustic collapse pressure function f(p) can be derived from the acoustic collapse curve by fitting the data with a sigmoid function such as a Boltzmann sigmoid function. An acoustic collapse pressure profile in the sense of the disclosure can include a set of initial collapse pressure values, a midpoint collapse pressure value and a set of complete collapse pressure values. The initial collapse pressures are the acoustic collapse pressures at which 5% or less of the GV signal is erased. A midpoint collapse pressure is the acoustic collapse pressure at which 50% of the GV signal is erased. Complete collapse pressures are the acoustic collapse pressures at which 95% or more of the GV signal is erased. The pressure can be peak pressure. In some embodiments, the peak pressure is peak positive pressure. In some embodiments, the peak pressure is peak negative pressure.

U.S. Patent Application Publication No. 2018/0030501 describes hybrid gas vesicle gene cluster (GVGC) configured for expression in a prokaryotic host comprising gas vesicle assembly (GVA) genes native to a GVA prokaryotic species and capable of being expressed in a functional form in the prokaryotic host, as well as one or more gas vesicle structural (GVS) genes native to one or more GVS prokaryotic species, at least one of the one or more GVS prokaryotic species different from the GVA prokaryotic species, and related gas vesicle reporting (GVR) genetic circuits, genetic, vectors, engineered cells, and related compositions methods and systems to produce GVs, hybrid GVGC and/or image a target site, the content of which is hereby expressly incorporated by reference in its entirety. The term “Gas Vesicle Genes Cluster” or “GVGC” as described herein indicates a gene cluster encoding a set of GV proteins capable of providing a GV upon expression within a cell. In some embodiments, the nucleic acid compositions provided herein encode some or all elements of a GVGC. The term “gene cluster” as used herein means a group of two or more genes found within an organism's DNA that encode two or more polypeptides or proteins, which collectively share a generalized function or are genetically regulated together to produce a cellular structure and are often located within a few thousand base pairs of each other. The size of gene clusters can vary significantly, from a few genes to several hundred genes. Portions of the DNA sequence of each gene within a gene cluster are sometimes found to be similar or identical; however, the resulting protein of each gene is distinctive from the resulting protein of another gene within the cluster. Genes found in a gene cluster can be observed near one another on the same chromosome or native plasmid DNA, or on different, but homologous chromosomes. An example of a gene cluster is the Hox gene, which is made up of eight genes and is part of the Homeobox gene family. In the sense of the disclosure, gene clusters as described herein also comprise gas vesicle gene clusters, wherein the expressed proteins thereof together are able to form gas vesicles.

Engineered GVs and methods of tuning the acoustic properties thereof are provided in U.S. Patent Application Publication No. 2020/0164095, the content of which is incorporated herein by reference in its entirety. In some embodiments, the GVs can be engineered to modulate the GV mechanical, acoustic, surface and targeting properties in order to achieve enhanced harmonic responses and multiplexed imaging to be better distinguished from background tissues. In some embodiments herein described Gas vesicles protein structures can be provided by Gyp genes endogenously expressed in bacteria or archaea. Endogenous expression can refer to expression of Gyp proteins forming the protein shell of the GV in bacteria or archaea that naturally produce gas vesicles encoded (e.g. in their genome or native plasmid DNA). Gyp proteins expressed by bacteria or archaea typically include two primary structural proteins, here also indicated as GvpA and GvpC, and several putative minor components and chaperones as would be understood by a person skilled in the art. In some embodiments, heterologously expressed Gyp proteins to provide a GV type have independently at least 50% sequence identity, preferably at least 80%, more preferably at least 90%, most preferably at least 95% sequence identity compared to a reference sequence of corresponding Gyp protein using one of the alignment programs described using standard parameters. In some embodiments, multiplexed imaging methods are provided. The term “multiplex” can refer to the presence of two or more distinct GVPS types, each of which exhibits an acoustic collapse and/or buckling pressure profile distinct from one another. The two or more distinct GVPSs can be derived from different organisms or variants of GVPSs from the same or different organisms (e.g., archaea).

Reporting Therapeutic Cells

Provided herein include compositions, methods, and systems for noninvasive monitoring of cell therapies. There are provided, in some embodiments, methods and compositions for monitoring blood glucose and the function of islet transplants with ultrasound. Transplants based on encapsulated engineered cells, such as stem cell (SC)-derived beta cells and beta-mimetic designer cells, have been intensively investigated as an alternative for beta cell replacement in treatment of T1D. However, those cells are usually dispersed throughout the peritoneum upon transplantation, making noninvasive and long-term graft monitoring extremely challenging. Ultrasound is a promising imaging modality for monitoring those engineered cells but it lacks the molecular specificity. The UGECIs provided herein enable the monitoring of the mass and function of the implanted cells, as well as the measurement of blood glucose with ultrasound. In some embodiments, and without being bound by any particular theory, this can rely on (1) the blood glucose level and the insulin secretion being tightly coupled with intracellular calcium in beta cells or beta-mimetic designer cells and (2) the capability of genetically manipulating those cells before transplantation. In some embodiments provided herein, beta-mimetic designer cells are engineered to stably express UGECIs and methods comprising the implantation the encapsulated cells in vivo are provided. In some embodiments, and without being bound by any particular theory, assuming the cells are functional, upon rise of blood glucose, it is expected to detect the cytosolic calcium concentration increase through ultrasound. In some embodiments, and without being bound by any particular theory, UGECIs can provide a certain level of baseline ultrasound contrast, one can also monitor the total mass of implants and the ratio of functional units to better analyze the long-term performance of the transplants.

There are provided, in some embodiments, reporting therapeutic cells. In some embodiments, the reporting therapeutic cell comprises: Ca²⁺-sensing gas vesicles (GVs) disclosed herein, wherein the reporting therapeutic cell is configured to treat a disease or disorder of a subject upon administration. The presence and/or functionality of the reporting therapeutic cells can be capable of being monitored in vivo by application of ultrasound (US). Monitoring the functionality of the reporting therapeutic cells in vivo can comprise detecting one or more Ca²⁺-coupled biological processes, optionally insulin secretion. The reporting therapeutic cell can be a replacement for a cell that is absent, diseased, infected, and/or involved in maintaining, promoting, or causing a disease or condition in a subject in need The disease can be a metabolic disease (e.g., T1D (type-1 diabetes), T2D (type-2 diabetes), diabetic ketoacidosis, obesity, cardiovascular disease, the metabolic syndrome and cancer). The reporting therapeutic cell can be autologous, allogenic, or xenogenic. The reporting therapeutic cell can be a stem cell (SC)-derived beta cell or a beta-mimetic designer cell. The reporting therapeutic cell can be capable of producing detectable ultrasound contrast in response to dynamic blood glucose levels and/or insulin secretion. There are provided, in some embodiments, methods of generating reporting therapeutic cells. In some embodiments, the method comprises: introducing a nucleic acid composition disclosed herein into engineered therapeutic cells to generate reporting therapeutic cells capable of expressing the one or more GVA protein(s), the one or more GVS protein(s), and the Ca²⁺-sensing GvpC protein. In some embodiments, the engineered therapeutic cell is a stem cell (SC)-derived beta cell or a beta-mimetic designer cell.

There are provided, in some embodiments, methods of monitoring a cell-based therapy. In some embodiments, the method comprises: administering to a subject an effective amount of the reporting therapeutic cells disclosed herein; and applying ultrasound (US) to a target site of the subject, thereby monitoring the cell-based therapy. The subject can be a subject suffering from a disease or disorder The disease can be a metabolic disease (e.g., T1D (type-1 diabetes), T2D (type-2 diabetes), diabetic ketoacidosis, obesity, cardiovascular disease, the metabolic syndrome and cancer). Monitoring the cell-based therapy can comprise monitoring the mass and/or function of the administered reporting therapeutic cells. Monitoring the functionality of the administered reporting therapeutic cells can comprise detecting one or more Ca²⁺-coupled biological processes, optionally insulin secretion. Monitoring the cell-based therapy can comprise determining the ratio of functional reporting therapeutic cells post-administration. The reporting therapeutic cells can be administered to a target site of the subject. The administering can comprise transplantation of the reporting therapeutic cells, optionally transplantation at one or more target sites. The target site can comprise a site of disease or disorder or can be proximate to a site of a disease or disorder. The cell-based therapy can be a transplant and/or tissue replacement. The administering can comprise implanting the reporting therapeutic cells into a target site of the subject. Monitoring the cell-based therapy can comprise monitoring blood glucose levels and/or insulin secretion at the target site.

Cell-Based Ultrasonic Biosensors of Extracellular Signals

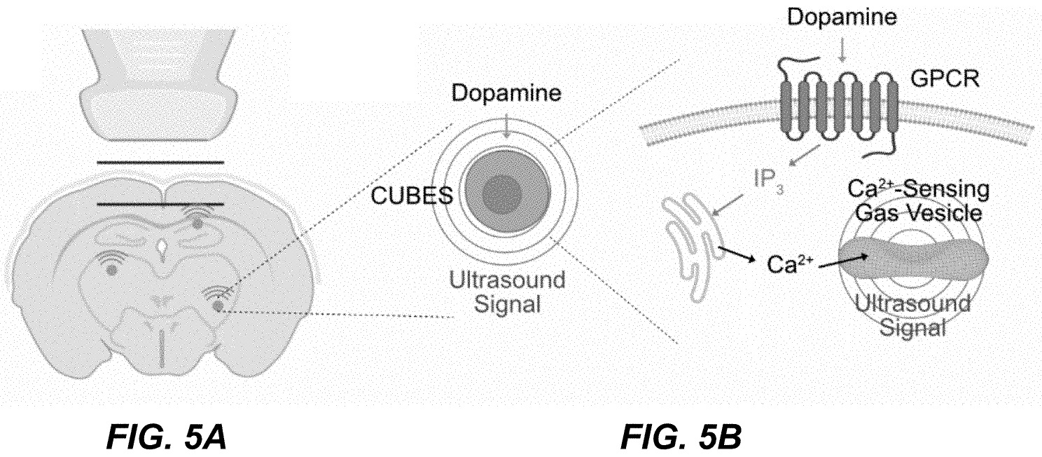

There are provided, in some embodiments, cell-based ultrasonic biosensors of extracellular signals (CUBES) (e.g., extracellular signal-sensing cells). In some embodiments, CUBES are genetically engineered cells equipped with: (1) a sensing receptor (e.g., GPCR) that can respond to specific extracellular signals such as neurotransmitters or hormones, leading to elevations of intracellular calcium, and (2) expression UGECIs—ultrasonic genetically encoded acoustic reporters that increase ultrasound contrast in response to calcium. In some embodiments, CUBES are the ultrasound analogue of CNiFERs, which are cells engineered to change their fluorescence in response to neurotransmitters such as acetylcholine (ACh), dopamine (DA) and norepinephrine (NE) or hormones such as somatostatin. CNiFERs have been implanted in vivo in the mouse brain and shown to sense neurotransmitters without causing major toxicity or disruption to brain tissue. There are provided, in some embodiments, methods and compositions comprising co-expression of GPCRs targeting different agonists (e.g. DA in FIGS. 5A-5B) and UGECIs in cells with low endogenous GPCR signaling (e.g. HEK293 cells). In some embodiments, said CUBES are implanted at desired anatomical locations where they sense their cognate extracellular signal and produce ultrasound contrast through UGECIs upon the elevation of intracellular calcium (FIGS. 5A-5B).

In some embodiments provided herein, CUBES are a versatile technology platform for non-invasive, wide-scale imaging of dynamic extracellular signals, which play key roles in virtually every tissue. For example, in neuroscience, the ability to visualize specific neurotransmitters such as DA will enable the study of circuits involved in learning and motivation and diseases such as Parkinson's and addiction. Moreover, CUBES can be combined with hemodynamic functional ultrasound to provide a simultaneous readout of broader brain-wide activity. In other tissues, by coupling UGECIs with different GPCRs, CUBES can be adapted to sense everything from hormones to chemokines, enabling the monitoring of metabolic, immune and other essential functions and diseases. Beyond applications in basic biology, CUBES can play a significant role in cell-based therapies such as transplants and tissue replacements. Incorporating CUBES into such transplants alongside therapeutic cells can enable noninvasive monitoring of the viability and functionality of the cellular therapies or their microenvironment via extracellular signals.

There are provided, in some embodiments, extracellular signal-sensing cells. In some embodiments, the extracellular signal-sensing cell comprises: a sensing receptor capable of binding an extracellular signal, wherein sensor receptor signaling triggered by said binding is capable of modulating intracellular Ca²⁺ levels; and Ca²⁺-sensing gas vesicles (GVs) disclosed herein. The extracellular signal-sensing cell can be capable of producing detectable ultrasound contrast in response to dynamic extracellular signals. The extracellular signal binding the sensing receptor can be capable of activating a sensing receptor signaling pathway leading to calcium release from the endoplasmic reticulum of the extracellular signal-sensing cell, thereby elevating intracellular Ca²⁺ levels. In some embodiments, the sensing receptor is, comprises, or is derived from, a G protein-coupled receptor (GPCR).

The GPCR can be a chemokine receptor, a cytokine receptor, class A GPCR, class B GPCR, a class C GPCR, a class D GPCR, a class E GPCR, and a class F GPCR, adhesion GPCR, frizzled GPCR, acetylcholine receptor, melatonin receptor, melacortin receptor, motilin receptor, Lysophospholipid (LP A) receptor), adenosine receptor, adreno receptor, angiotensin receptor, bradykinin receptor, calcitonin receptor, calcium sensing receptor, cannabinoid receptor, chemokine receptor, cholecystokinin receptor, complement component (C5AR1) receptor, corticotrophin releasing factor receptor, dopamine receptor, endothelial differentiation gene receptor, endothelin receptor, formyl peptide-like receptor, galanin receptor, gastrin releasing peptide receptor, receptor ghrelin receptor, gastric inhibitory polypeptide receptor, glucagon receptor, gonadotropin releasing hormone receptor, histamine receptor, kisspeptin (KiSS1) receptor, leukotriene receptor, melanin-concentrating hormone receptor, melanocortin receptor, melatonin receptor, motilin receptor, neuropeptide receptor, nicotinic acid, opioid receptor, orexin receptor, orphan receptor, platelet activating factor receptor, prokineticin receptor, prolactin releasing peptide, prostanoid receptor, protease activated receptor, P2Y (purinergic) receptor, relaxin receptor, secretin receptor, serotonin receptor, somatostatin receptor, tachykinin receptor, vasopressin receptor, oxytocin receptor, vasoactive intestinal peptide (VIP) receptor or the pituitary adenylate cyclase activating polypeptide (PACAP) receptor, taste 1 receptor, metabotropic glutamate receptor, calcium-sensing receptor, brain specific angiogenesis inhibitor receptor (1, 2 or 3), cadherein receptor, an estrogen receptor, or any combination thereof. The extracellular signal can be one or more of a polypeptide, a peptide, a nucleotide, a growth factor, ahormone, a pheromone, a chemokine, a cytokine, a neurotransmitter, a lipid, and a sugar. The hormone can be selected from the group comprising thyroid-stimulating hormone, a follicle-stimulating hormone, a leuteinizing hormone, prolactin, growth hormone, adrenocorticotropic hormone, antidiuretic hormone, oxytocin, thyrotropin-releasing hormone, gonadotropin-releasing hormone, growth hormone-releasing hormone, corticotropin-releasing hormone, somatostatin, dopamine, melatonin, thyroxine, calcitonin, parathyroid hormone, a glucocorticoid, a mineralocorticoid, an androgen, adrenaline, an estrogen, progesterone, human chorionic gonadotropin, insulin, glucagons, somatostatin, erythropoietin, calcitriol, atrial-natriuretic peptide, gastrin, secretin, cholecystokinin, somatostatin, neuropeptide Y, ghrelin, PYY3-36, insulin-like growth factor-1, angiotensinogen, thrombopoietin, leptin, or any combination thereof.

The neurotransmitter can be selected from the group comprising acetylcholine, epinephrine (adrenaline), norepinephrine (noradrenaline), dopamine, 5-hydroxytryptamine (serotonin), glutamic acid, L-3,4-dihydroxyphenylalanine (L-dopa), 3,4-Dihydroxyphenylacetic acid (DOPAC), homovannilic acid, tyramine, or any combination thereof. The neurotransmitter can be a neuroactive peptide (e.g., bradykinin, cholecystokinin, gastrin, secretin, oxytocin, a sleep peptide, gonadotropin-releasing hormone, beta-endorphin, enkephalin, substance P, somatostatin, prolactin, galanin, growth hormone-releasing hormone, bombesin, dynorphin, neurotensin, motilin, thyrotropin, neuropeptide Y, leuteinizing hormone, calcitonin, or vasoactive intestinal peptide, or any combination thereof).

The extracellular signal-sensing cell can be capable of dynamically sensing extracellular signals upon administration to a subject. In some embodiments, said administration can comprise transplantation, optionally transplantation into one or more target brain region(s). The extracellular signal-sensing cell can comprise one or more secondary sensing receptor(s), wherein the sensor receptor and the secondary sensing receptor(s) target different agonists and/or bind different extracellular signals. The extracellular signal-sensing cell can be capable of dynamically sensing changes in the concentration of the extracellular signal via changes in nonlinear ultrasound contrast. The extracellular signal-sensing cells can be implanted within a target site of a subject, and wherein applying US comprises applying US to the target site to obtain a US image of the target site. In some embodiments, an at least about 5 dB, about 4 dB, about 3 dB, about 2 dB, about 1 dB, about 0.8 dB, about 0.6 dB, about 0.4 dB, about 0.2 dB, about 0.1 dB, about 0.05 dB, or about 0.01 dB, enhancement in nonlinear ultrasound contrast indicates the presence of the extracellular signal.

In some embodiments, administration of the effective amount of extracellular signal-sensing cells to a subject does not cause neurological side effects, toxicity, and/or disruption of physiological functions of the subject. In some embodiments, in the absence of the extracellular signal, the sensing receptor can have low or no endogenous activity. The extracellular signal-sensing cell can have low endogenous signaling of the sensing receptor, optionally less than 10 percent of maximal signaling of the sensing receptor upon binding the extracellular signal. The amount of ultrasound contrast can be correlated with the amount of extracellular signal at the target site.

There are provided, in some embodiments, methods of monitoring extracellular signal dynamics. In some embodiments, the method comprises: administering to a subject an effective amount of the extracellular signal-sensing cells disclosed herein; and applying ultrasound (US) to a target site of the subject, thereby monitoring extracellular signal dynamics. In some embodiments, applying US causes the Ca²⁺-sensing GVs to produce an extracellular signal-dependent nonlinear ultrasound contrast.

There are provided, in some embodiments, methods of monitoring a cell-based therapy. In some embodiments, the method comprises: administering to a subject an effective amount of the extracellular signal-sensing cells disclosed herein; administering to the subject an effective amount of a cell-based therapy; and applying ultrasound (US) to a target site of the subject, thereby monitoring the cell-based therapy. The performance of the cell-based therapy can be associated with extracellular signal dynamics. Monitoring the cell-based therapy can comprise monitoring the viability and/or functionality of the cell-based therapy via monitoring extracellular signal dynamics. The viability and/or functionality of the cell-based therapy can be associated with and/or caused by extracellular signal dynamics. Monitoring the cell-based therapy can comprise monitoring the microenvironment of the cell-based therapy via monitoring extracellular signal dynamics. The extracellular signal-sensing cells and/or cell-based therapy can be administered to a target site of the subject. The extracellular signal-sensing cells and cell-based therapy can be co-administered. The extracellular signal-sensing cells can be incorporated into the cell-based therapy. The administering can comprise transplantation, optionally transplantation at one or more target sites. The target site can comprise a site of disease or disorder or can be proximate to a site of a disease or disorder, optionally the target site(s) comprises one or more target brain region(s). The cell-based therapy can be a transplant and/or tissue replacement.

There are provided, in some embodiments, methods of monitoring a neural circuit of the subject. In some embodiments, the method comprises: administering to a subject an effective amount of the extracellular signal-sensing cells disclosed herein; and applying ultrasound (US) to a target site of the subject, thereby monitoring a neural circuit of the subject. The activity of the neural circuit can be caused by and/or associated with extracellular signal dynamics and/or Ca²⁺-associated biological processes. The neural circuit can be associated with a disease or disorder, e.g., one or more of schizophrenia, drug craving, drug addiction, bipolar disorder, anxiety, depression, Parkinson's disease, Alzheimer's disease, cognitive dysfunction, multiple sclerosis, Amyotrophic lateral sclerosis (ALS), ischemic stroke, HIV dementia, and Huntington's disease. The neural circuit can be associated with a behavior or physiological function, e.g., one or more of learning, motivation, memory, attention, concentration, alertness, mental flexibility and/or speed, learning, intelligence, language skills, problem solving capacity, consciousness, coping with psychological stress or tension, motivation, mobility, decision making capacity, reaction time, and regulation of emotions.

There are provided, in some embodiments, methods of monitoring a physiological function of a subject. In some embodiments, the method comprises: administering to a subject an effective amount of the extracellular signal-sensing cells disclosed herein; and applying ultrasound (US) to a target site of the subject, thereby monitoring the physiological function of the subject. The physiological function can be caused by and/or associated with extracellular signal dynamics and/or Ca²⁺-associated biological processes. The physiological function can comprise an immune function or metabolic function of the subject.

There are provided, in some embodiments, methods of identifying or monitoring a disease or disorder. In some embodiments, the method comprises: administering to a subject an effective amount of the extracellular signal-sensing cells disclosed herein; and applying ultrasound (US) to a target site of the subject, thereby identifying or monitoring the disease or disorder.

The disease or disorder can be caused by and/or associated with extracellular signal dynamics and/or Ca²⁺ associated biological processes. The disease or disorder can be a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. The disease or disorder can comprise a neurological disease or disorder.

The neurological disease or disorder can comprise epilepsy, Dravet Syndrome, Lennox Gastaut Syndrome, mycolonic seizures, juvenile mycolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

Imaging Methods

There are provided, in some embodiments, methods of generating Ca²⁺-sensing gas vesicles (GVs). In some embodiments, the method comprises: introducing a nucleic acid composition disclosed herein into host cells capable of expressing the one or more GVA protein(s), the one or more GVS protein(s), and the Ca²⁺-sensing GvpC protein to generate the Ca²⁺-sensing gas vesicles (GVs). In some embodiments, the host cell is a prokaryotic cell or a eukaryotic cell.

There are provided, in some embodiments, methods of generating Ca²⁺-sensing gas vesicles (GVs). In some embodiments, the method comprises: providing gas vesicles (GVs) comprising a naturally occurring GvpC protein; removing the naturally occurring GvpC protein from said GVs to generate stripped GVs, optionally via urea; and contacting the stripped GVs with a Ca²⁺-sensing GvpC protein disclosed herein, thereby generating Ca²⁺-sensing gas vesicles (GVs).

There are provided, in some embodiments, methods of imaging a target site of a subject. In some embodiments, the method comprises: obtaining a subject comprising the Ca²⁺-sensing gas vesicles (GVs) disclosed herein; and applying ultrasound (US) to the target site of the subject to obtain a US image of the target site, optionally a nonlinear US image. In some embodiments, the obtaining comprises introducing a nucleic acid composition disclosed herein into one or more target cells of said subject.

There are provided, in some embodiments, methods of imaging intracellular Ca²⁺ dynamics. In some embodiments, the method comprises: obtaining target cells comprising the Ca²⁺-sensing gas vesicles (GVs) disclosed herein; and applying ultrasound (US) to said target cells, thereby imaging intracellular Ca²⁺ dynamics. In some embodiments, the obtaining comprises introducing a nucleic acid composition disclosed herein into the target cells, optionally target cells of a subject.

There are provided, in some embodiments, methods of monitoring Ca²⁺-associated biological processes. In some embodiments, the method comprises: obtaining target cells comprising the Ca²⁺-sensing gas vesicles (GVs) disclosed herein; and applying ultrasound (US) to said target cells, thereby monitoring Ca²⁺-associated biological processes. In some embodiments, the obtaining comprises introducing a nucleic acid composition disclosed herein into the target cells, optionally target cells of a subject.

There are provided, in some embodiments, methods of detecting perturbation-induced changes in intracellular Ca²⁺ dynamics. In some embodiments, the method comprises: obtaining target cells comprising the Ca²⁺-sensing gas vesicles (GVs) disclosed herein; introducing one or more genetic, chemical, and/or physical perturbations to said target cells; and applying ultrasound (US) to said target cells, thereby detecting the perturbation-induced changes in intracellular Ca²⁺ dynamics. The obtaining can comprise introducing a nucleic acid composition disclosed herein into the target cells, optionally target cells of a subject.

The term “contrast enhanced imaging” or “imaging”, as used herein indicates a visualization of a target site performed with the aid of a contrast agent administered to the target site to improve the visibility of structures or fluids by devices process and techniques suitable to provide a visual representation of a target site. Accordingly a contrast agent is a substance that enhances the contrast of structures or fluids within the target site, producing a higher contrast image for evaluation.

The term “ultrasound imaging” or ultrasound scanning” or “sonography” as used herein indicate imaging performed with techniques based on the application of ultrasound. Ultrasound can refer to sound with frequencies higher than the audible limits of human beings, typically over 20 kHz. Ultrasound devices typically can range up to the gigahertz range of frequencies, with most medical ultrasound devices operating in the 1 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a skilled person.

Accordingly, the wording “ultrasound imaging” as used herein can refer to in particular to the use of high frequency sound waves, typically broadband waves in the megahertz range, to image structures in the body. The image can be up to 3D with ultrasound. In particular, ultrasound imaging typically involves the use of a small transducer (probe) transmitting high-frequency sound waves to a target site and collecting the sounds that bounce back from the target site to provide the collected sound to a computer using sound waves to create an image of the target site. Ultrasound imaging allows detection of the function of moving structures in real-time. Ultrasound imaging works on the principle that different structures/fluids in the target site will attenuate and return sound differently depending on their composition. A contrast agent sometimes used with ultrasound imaging are microbubbles created by an agitated saline solution, which works due to the drop in density at the interface between the gas in the bubbles and the surrounding fluid, which creates a strong ultrasound reflection. Ultrasound imaging can be performed with conventional ultrasound techniques and devices displaying 2D images as well as three-dimensional (3-D) ultrasound that formats the sound wave data into 3-D images. In addition to 3D ultrasound imaging, ultrasound imaging also encompasses Doppler ultrasound imaging, which uses the Doppler Effect to measure and visualize movement, such as blood flow rates. Types of Doppler imaging includes continuous wave Doppler, where a continuous sinusoidal wave is used; pulsed wave Doppler, which uses pulsed waves transmitted at a constant repetition frequency, and color flow imaging, which uses the phase shift between pulses to determine velocity information which is given a false color (such as red=flow towards viewer and blue=flow away from viewer) superimposed on a grey-scale anatomical image. Ultrasound imaging can use linear or non-linear propagation depending on the signal level. Harmonic and harmonic transient ultrasound response imaging can be used for increased axial resolution, as harmonic waves are generated from non-linear distortions of the acoustic signal as the ultrasound waves insonate tissues in the body. Other ultrasound techniques and devices suitable to image a target site using ultrasound would be understood by a skilled person.

The basic physics of sound waves enables ultrasound to visualize biological tissues with high spatial and temporal resolution. This capability has been enhanced by the development of acoustic biomolecules—proteins with physical properties enabling them to scatter sound. The first acoustic biomolecules developed as contrast agents in ultrasound imaging, analogous to GFPs used in optical imaging, were based on a unique class of air-filled protein nanostructures called gas vesicles (GVs). The advancement of GVs has made it possible to use ultrasound to visualize the functions of cells deep inside tissues.

The term “ultrasound” can refer to sound with frequencies higher than the audible limits of human beings, typically over 20 kHz. Ultrasound devices typically can range up to the gigahertz range of frequencies, with most medical ultrasound devices operating in the 0.2 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a person skilled in focused ultrasound. U.S. Patent Application Publication No. 2020/0237346 describes methods comprising the application of a step function increase in acoustic pressure during ultrasound imaging using gas vesicle contrast, along with capturing successive frames of ultrasound imaging and extracting time-series vectors for pixels of the frames, the content of which is hereby expressly incorporated by reference in its entirety. In some embodiments, the first, second, third, fourth, fifth, and/or sixth US pulse(s) each comprise a set of pulses.

Focused ultrasound (“FUS”) can refer to the technology that uses ultrasound energy to target specific areas of a subject, such as a specific area of a brain or body. FUS focuses acoustic waves by employing concave transducers that usually have a single geometric focus, or an array of ultrasound transducer elements which are actuated in a spatiotemporal pattern such as to produce one or more focal zones. At this focus or foci most of the power is delivered during sonication in order to induce mechanical effects, thermal effects, or both. The frequencies used for focused ultrasound are in the range of 200 KHz to 8000 KHz.

As used herein, the term “harmonic signal” or “harmonic frequency” can refer to a frequency in a periodic waveform that is an integer multiple of the frequency of the fundamental signal. In addition, this term encompasses sub-harmonic signals, which are signals with a frequency equal to an integral submultiple of the frequency of the fundamental signal. In ultrasound imaging, the transmitted pulse is typically centered around a fundamental frequency, and received signals may be processed to isolate signals centered around the fundamental frequency or one or more harmonic frequencies.

The term “fundamental signal” or “fundamental wave” can refer to the primary frequency of the transmitted ultrasound pulse. All GVs can backscatter ultrasound at the fundamental frequency, allowing their detection by ultrasound.

The term “non-linear signal” can refer to a signal that does not obey superposition and scaling properties, with regards to the input. The term “linear signal” can refer to a signal that does obey those properties. One example of non-linearity is the production of harmonic signals in response to ultrasound excitation at a certain fundamental frequency. Another example is a non-linear response to acoustic pressure. One embodiment of such a non-linearity is the acoustic collapse profile of GVs, in which there is a non-linear relationship between the applied pressure and the disappearance of subsequent ultrasound contrast from the GVs as they collapse. Another example of a non-linear signal that does not involve the destruction of GVs, is the increase in both fundamental and harmonic signals with increasing pressure of the transmitted imaging pulse, wherein certain GVs exhibit a super-linear relationship between these signals and the pulse pressure.

The term “applying ultrasound” shall be given its ordinary meaning, and shall also refer to sending ultrasound-range acoustic energy to a target. The sound energy produced by the piezoelectric transducer can be focused by beamforming, through transducer shape, lensing, or use of control pulses. The soundwave formed is transmitted to the body, then partially reflected or scattered by structures within a body; larger structures typically reflecting, and smaller structures typically scattering. The return sound energy reflected/scattered to the transducer vibrates the transducer and turns the return sound energy into electrical signals to be analyzed for imaging. The frequency and pressure of the input sound energy can be controlled and are selected based on the needs of the particular imaging/delivery task and, in some methods described herein, collapsing GVs. To create images, particularly 2D and 3D imaging, scanning techniques can be used where the ultrasound energy is applied in lines or slices which are composited into an image.

In certain embodiments, the method includes applying a set of imaging pulses from an ultrasound transmitter to the target site, and receiving ultrasound signal at a receiver. In certain instances, the ultrasound signal detected by the receiver includes an ultrasound echo signal. Additional information of ultrasound systems and methods can be found in related publications as will be understood by a person skilled in the art.

Methods for performing ultrasound imaging are known in the art and can be employed in methods of the current disclosure. In certain aspects, an ultrasound transducer, which comprises piezoelectric elements, transmits an ultrasound imaging signal (or pulse) in the direction of the target site. Variations in the acoustic impedance (or echogenicity) along the path of the ultrasound imaging signal causes backscatter (or echo) of the imaging signal, which is received by the piezoelectric elements. The received echo signal is digitized into ultrasound data and displayed as an ultrasound image. Conventional ultrasound imaging systems comprise an array of ultrasonic transducer elements that are used to transmit an ultrasound beam, or a composite of ultrasonic imaging signals that form a scan line. The ultrasound beam is focused onto a target site by adjusting the relative phase and amplitudes of the imaging signals. The imaging signals are reflected back from the target site and received at the transducer elements. The voltages produced at the receiving transducer elements are summed so that the net signal is indicative of the ultrasound energy reflected from a single focal point in the subject. An ultrasound image is then composed of multiple image scan lines.

In some embodiments, imaging the target site is performed by applying or transmitting an imaging ultrasound signal from an ultrasound transmitter to the target site and receiving a set of ultrasound data at a receiver. The ultrasound data can be obtained using a standard ultrasound device, or can be obtained using an ultrasound device configured to specifically detect the contrast agent used. Obtaining the ultrasound data can include detecting the ultrasound signal with an ultrasound detector. In some embodiments, the imaging step further comprises analyzing the set of ultrasound data to produce an ultrasound image.

In certain embodiments, the ultrasound signal has a transmit frequency of at least 1 MHz, 5 MHz, 10 MHz, 20 MHz, 30 MHz, 40 MHz or 50 MHz. For example, an ultrasound data is obtained by applying to the target site an ultrasound signal at a transmit frequency from 4 to 11 MHz, or at a transmit frequency from 14 to 22 MHz. In some instances, the imaging frequency is selected so as to maximize the contrast generated by the administered contrast agent. In some embodiments, applying ultrasound (US) to the target site of the subject comprises applying US to a plurality of target sites of the subject.

In the embodiments herein described, the collapsing ultrasound and imaging ultrasound are selected to have a collapsing pressure and an imaging pressure amplitude based on the acoustic collapse pressure profile (e.g., peak acoustic pressure) of the GVPS type used. In some instances, the ultrasound pressure, including the collapsing ultrasound pressure and the imaging ultrasound pressure can be referred to as the “peak positive pressure” of the ultrasound pulses. The term “peak positive pressure” can refer to the maximum pressure amplitude of the positive pulse of a pressure wave, typically in terms of the difference between the peak pressure and the ambient pressure at the location in the person or specimen that is being imaged.

In some embodiments, target cells can be situated within a target site of a subject, and wherein applying US comprises applying US to the target site to obtain a US image of the target site. The target cells can be situated within a plurality of target sites of a subject, and wherein applying US comprises applying US to the plurality of target sites to obtain US images of the plurality of target sites. Applying US to the target site can comprise applying US to a plurality of target sites of a subject. In some embodiments, applying US causes the Ca²⁺-sensing GVs to produce a Ca²⁺-dependent nonlinear ultrasound contrast. In some embodiments, an at least about 5 dB, about 4 dB, about 3 dB, about 2 dB, about 1 dB, about 0.8 dB, about 0.6 dB, about 0.4 dB, about 0.2 dB, about 0.1 dB, about 0.05 dB, or about 0.01 dB, enhancement in nonlinear ultrasound contrast indicates the presence of intracellular Ca²⁺ and/or a Ca²⁺-associated biological processes.

Applying US can comprise applying hemodynamic functional US. Applying US can comprise nonlinear US imaging. Applying US can comprise applying one or more US pulse(s) over a duration of time. The duration of time can be about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the one or more US pulse(s) each have a pulse duration of about 1 hour, about 30 minutes, about 15 minutes, about 10 minutes, about 5 minutes, about 1 minute, about 1 second, or about 1 millisecond. Applying one or more US pulse(s) can comprise applying one or more focused US pulse(s). Applying one or more US pulse(s) can comprise applying US at a frequency of 100 kHz to 100 MHz. Applying one or more US pulse(s) can comprise applying ultrasound having a mechanical index in a range between 0.2 and 0.6. The one or more US pulse(s) can comprise a peak pressure of about 40 kPa to about 1500 kPa. The one or more US pulse(s) can comprise a pressure value that is: the selectable buckling pressure; the first optimum buckling pressure; and/or selected from the first buckling pressure profile that optimally maximizes buckling of the Ca²⁺-sensing GV in a GV soft state while minimizing buckling of Ca²⁺-sensing GV in a GV stiff state. In some embodiments, the one or more US pulse(s) induces collapse of Ca²⁺-sensing GV in a GV soft state, and wherein the one or more US pulse(s) comprise a pressure value that is: the first selectable collapse pressure; the second selectable collapse pressure; and/or selected from the second collapse pressure profile that optimally maximizes collapse of the Ca²⁺-sensing GV in a GV soft state while minimizing collapse of Ca²⁺-sensing GV in a GV stiff state.

The nonlinear ultrasound imaging can comprise cross-amplitude modulation (x-AM) ultrasound imaging or parabolic amplitude modulation (pAM) ultrasound imaging. The nonlinear ultrasound imaging can comprise differential nonlinear ultrasound imaging. In some embodiments, differential nonlinear ultrasound imaging can comprise imaging of the second and/or higher harmonics with the first harmonic signal subtracted out. The method can comprise cross-phase modulation imaging and/or harmonic imaging. The nonlinear ultrasound imaging can comprise providing amplitude modulation (AM) ultrasound pulse sequences in order to image and differentiate the baseline nonlinear behavior of buckling Ca²⁺-sensing GV in a GV stiff state from the increased nonlinear behavior of buckling Ca²⁺-sensing GV in a GV soft state. In some embodiments, the nonlinear ultrasound imaging comprises: pairs of cross-propagating plane waves to elicit nonlinear scattering from buckling Ca²⁺-sensing GVs at the wave intersection; subtracting the signal generated by transmitting each wave on its own; and quantifying the resulting contrast. The signals generated by transmitting each wave on its own can have linear characteristics and/or lower nonlinear characteristics than the combined transmission of both plane waves produced at their intersection. In some embodiments, the nonlinear ultrasound imaging comprises: a peak positive pressure of two single tilted plane waves exciting the Ca²⁺-sensing GV in a linear scattering regime; a doubled X-wave intersection amplitude exciting the Ca²⁺-sensing GV in a nonlinear scattering regime; summing the echoes from the two single tilted plane-wave transmissions to generate a sum; and subtracting the sum from the echoes of the X-wave transmissions to derive nonzero differential Ca²⁺-sensing GV signals.

Applying US can comprise detecting scattering of the one or more US pulse(s) by Ca²⁺-sensing GV. Applying US can comprise detecting increased nonlinear scattering of the US by buckling Ca²⁺-sensing GV in a GV soft state. In some embodiments, detecting scattering comprises: detecting backscattered echoes of two half-amplitude transmissions at applied pressures below the buckling threshold of the Ca²⁺-sensing GV. In some embodiments, said two half-amplitude transmissions trigger largely linear scattering. In some embodiments, detecting backscattered echoes of a third full-amplitude transmission at pressures above the buckling threshold of the Ca²⁺-sensing GV. In some embodiments, said third full-amplitude transmission triggers harmonic and nonlinear scattering. The method can comprise subtracting the backscattered echoes of the two half-amplitude transmissions from the backscattered echoes of the third full-amplitude transmission.

In some embodiments, the method comprises: single-cell Ca²⁺ imaging; and/or imaging a large volume in deep tissue. The method can comprise US imaging with a spatiotemporal resolution of less than about 100 μm (e.g., 100 μm, 10 μm, 1 μm, 0.1 μm, 0.01 μm, 0.001 μm or a range between any two of these values) and less than about 1 ms (e.g., 1 ms, 0.1 ms, 0.01 ms, 0.001 ms, or a range between any two of these values). In some embodiments, the target site comprises: a volume larger than about 1 mm³ (e.g., 1 mm³, 10 mm³, 100 mm³, 1000 mm³, 10000 mm³, 100000 mm³, or a range between any two of these values); a depth deeper than about 1 mm; (e.g., 1 mm, 10 mm, 100 mm, 1000 mm, 10000 mm, 100000 mm, or a range between any two of these values); a depth and/or a volume inaccessible via optical imaging and/or fiber photometry; and/or the entire brain or a portion thereof.

The target site can comprise target cells. The target cells can comprise excitable cells, optionally the biological processes comprise one or more of Ca²⁺-coupled neural activities, synaptic plasticity, and brain electrical signaling. The target cells can comprise non-excitable cells, optionally the biological processes comprise T-cell activation and insulin secretion from beta cells. The target cells can be in vitro, in vivo, and/or ex vivo, optionally the target cells are tissue culture cells. The subject can be a mammal. In some embodiments, the subject is not anesthetized. The target site can comprise a site of disease or disorder or can be proximate to a site of a disease or disorder.

The target site can comprise a tissue, optionally the tissue is inflamed tissue and/or infected tissue. The tissue can comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the tissue comprises: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue.

The target site can comprise one or more target brain region(s). The brain region can comprise neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof.

The target brain region(s) can comprise the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (S1), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof.

The administering can comprise systemic administration. The systemic administration can be intravenous, intramuscular, intraperitoneal, or intraarticular. Administering can comprise intracranial delivery, intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The period of time between the administering and applying US can be about 21 days, about 14 days, about 7 days, about 3 days, about 48 hours, about 44 hours, about 40 hours, about 35 hours, about 30 hours, about 25 hours, 20 hours, 15 hours, 10 hours, about 8 hours, about 7 hours, about 6 hours, about 5 hours, about 4 hours, about 3 hours, about 2 hours, about 1 hour, about 30 minutes, about 15 minutes, about 10 minutes, or about 5 minutes. An effective amount of the engineered cells (e.g., reporting therapeutic cells extracellular signal-sensing cells) can be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the effective amount of the engineered cells (e.g., reporting therapeutic cells extracellular signal-sensing cells) is about 10⁴ cells, about 10⁵ cells, about 10⁶ cells, about 10⁷ cells, or about 10⁸ cells. In one particular embodiment, the effective amount of the engineered cells (e.g., reporting therapeutic cells extracellular signal-sensing cells) is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the administering step comprises administering a vector (e.g., viral vector) comprising a nucleic acid composition provided herein. The compositions (e.g., nucleic acid compositions, reporting therapeutic cells extracellular signal-sensing cells) described herein may be included in a composition for administration. The composition may include a pharmaceutical composition and further include a pharmaceutically acceptable carrier.

### EXAMPLES

Some aspects of the embodiments discussed above are disclosed in further detail in the following examples, which are not in any way intended to limit the scope of the present disclosure.

### Example 1

### Ultrasonic Genetically Encoded Calcium Indicators

Summary

Disclosed herein include UGECIs (e.g., Ca²⁺-sensing GvpC proteins, Ca²⁺-sensing gas vesicles). These first-of-a-kind molecular tools are based on gas vesicles (GVs), a unique class of air-filled protein nanostructures (FIGS. 1A-1B) encoded in the genomes of certain buoyant bacteria, which can scatter sound waves and thereby produce ultrasound contrast. Just as GFP, cloned from jellyfish, was the first protein visible with fluorescence microscopy, GVs are the first protein visible with ultrasound. Genetic constructs enabling the heterologous expression of GVs as reporter genes for in vivo imaging of bacterial and mammalian gene expression (FIGS. 1C-1D), established methods to genetically engineer the acoustic properties of GVs, and ultrasound pulse sequences to take advantage of these properties for maximal sensitivity are provided herein. There is provided, in some embodiments, the first generation of UGECIs: genetically engineered GVs that reversibly change their acoustic contrast in response to Ca²⁺. Ultrasound imaging of intracellular calcium dynamics can be implemented with the UGECIs provided herein. Additionally, monitoring of calcium coupled cellular functions in vivo can be performed with the UGECIs provided herein, especially in a large imaging volume and deep regions where using optical imaging might be challenging.

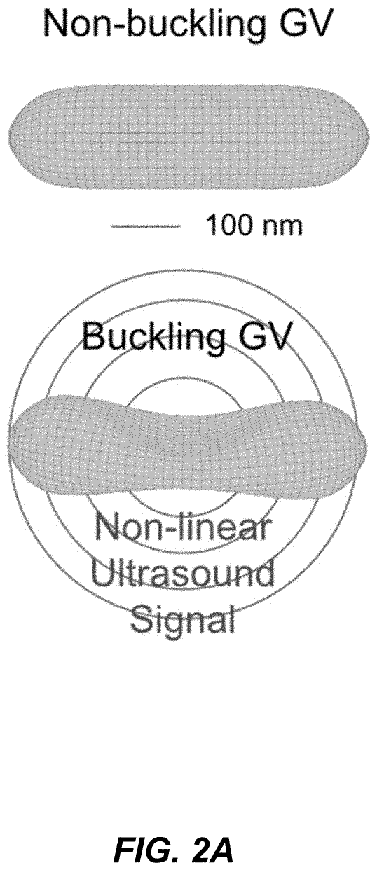

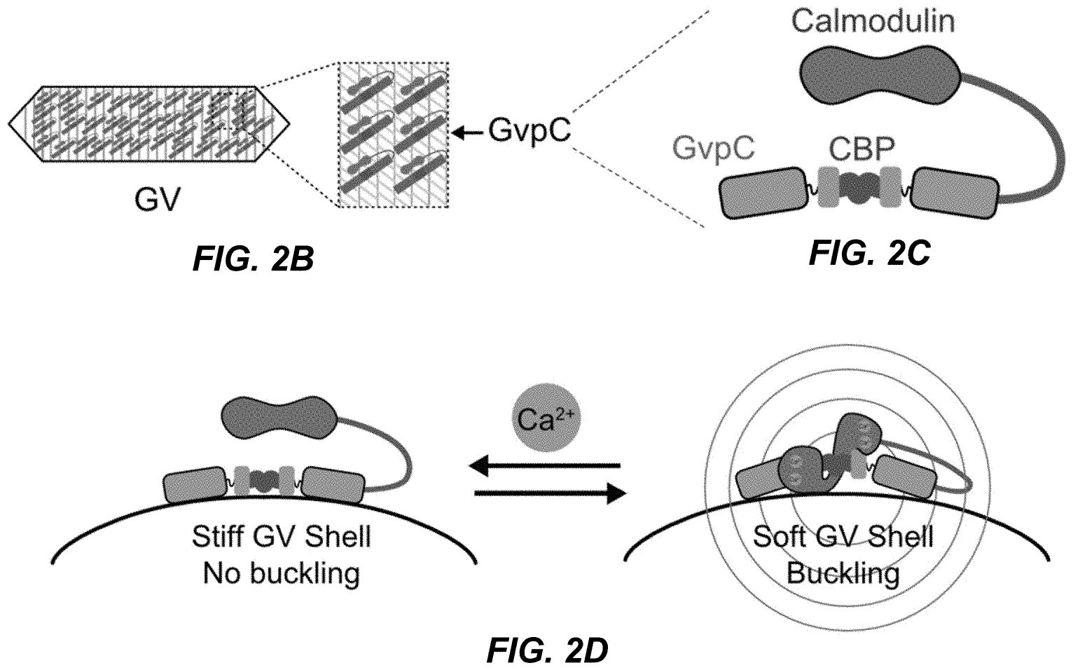

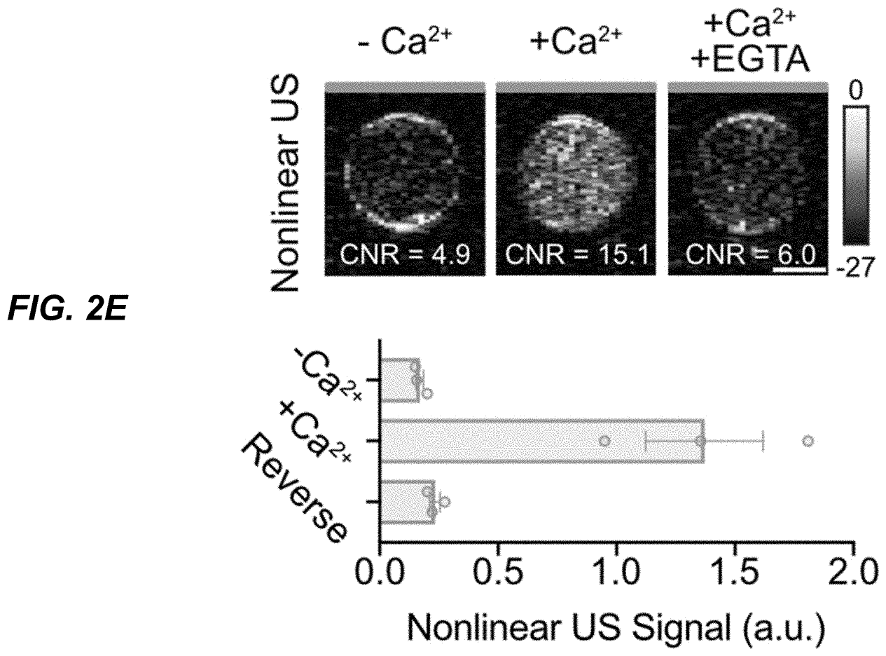

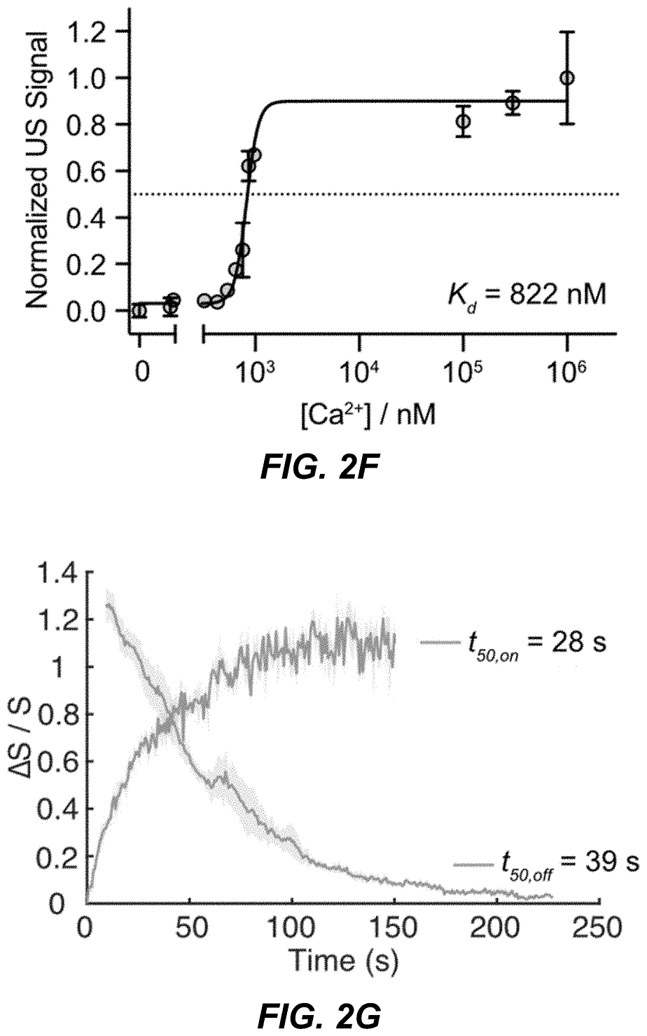

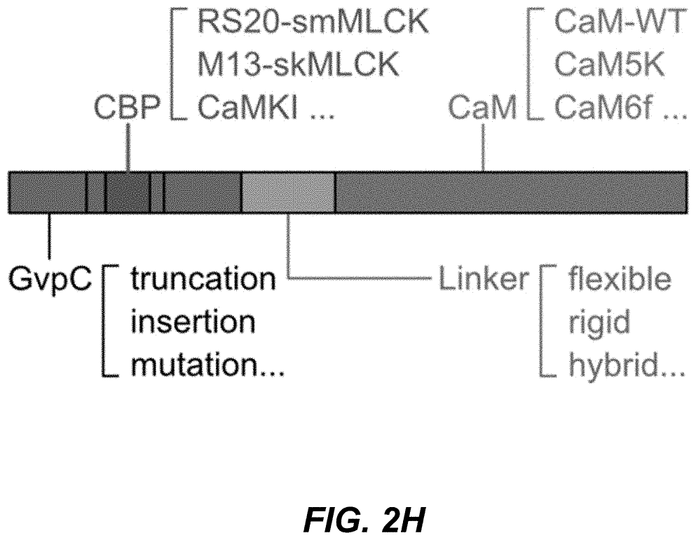

In some embodiments, and without being bound by any particular theory, the basic design of UGECIs can rely on the fact that GVs with a softer protein shell undergo larger “buckling” deformations in response to ultrasound pulses (FIG. 2A), resulting in the production of non-linear ultrasound signals, which can be selectively imaged with conventional ultrasound devices. In some embodiments, an alpha-helical protein called GvpC, bound to the GV shell surface, acts as a mechanical stiffener, controlling the extent of GV buckling and nonlinear contrast (FIG. 2B). In some embodiments, of the methods and compositions provided herein, the GvpC protein is engineered to develop acoustic biosensors.

To develop UGECIs, GvpC was modified to incorporate a calcium binding motif (FIG. 2C). Upon Ca²⁺ binding, this engineered GvpC can undergo an allosteric conformational change resulting in reduced GV shell stiffness, increased buckling, and the appearance of non-linear signal (FIG. 2D). Unlike protease sensor GvpC proteins, UGECIs can be fully reversible. This biosensor design was implemented in purified GVs from the cyanobacterium Anabaena flos-aquae. This proof-of-concept biosensor incorporates the CBP from CaMKI in the second repeat of a 3-repeat GvpC that is truncated from the 5-repeat version natively expressed in Anabaena flos-aquae. It has a mutant human CaM attached at its C terminus via a long flexible linker (FIG. 2C). The first generation of UGECI produces reversible non-linear ultrasound contrast in response to Ca²⁺ (FIG. 2E), with half-maximal contrast occurring at 822 nM (FIG. 2F). This first generation of UGECIs showed a sub-minute kinetics with both half-rise and half-decay time below 40 seconds (FIG. 2G).

In addition to the demonstrated version of UGECI, several aspects of the design can be tuned to alter performance of UGECIs, such as calcium affinity and response kinetics. Non-limiting exemplary engineerable components of the UGECI design (FIG. 211) include the CBP, the CaM, the location and linkers integrating the CBP into GvpC and the linker used to append CaM. Over 100 variants were tested using these strategies and different versions of UGECIs can be employed for various applications.

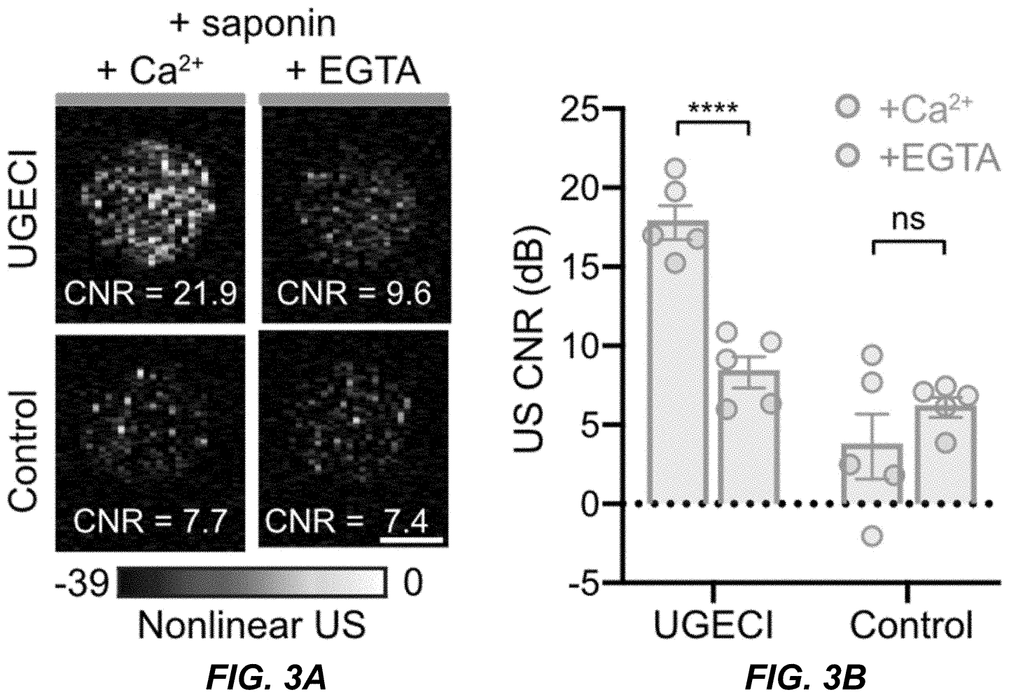

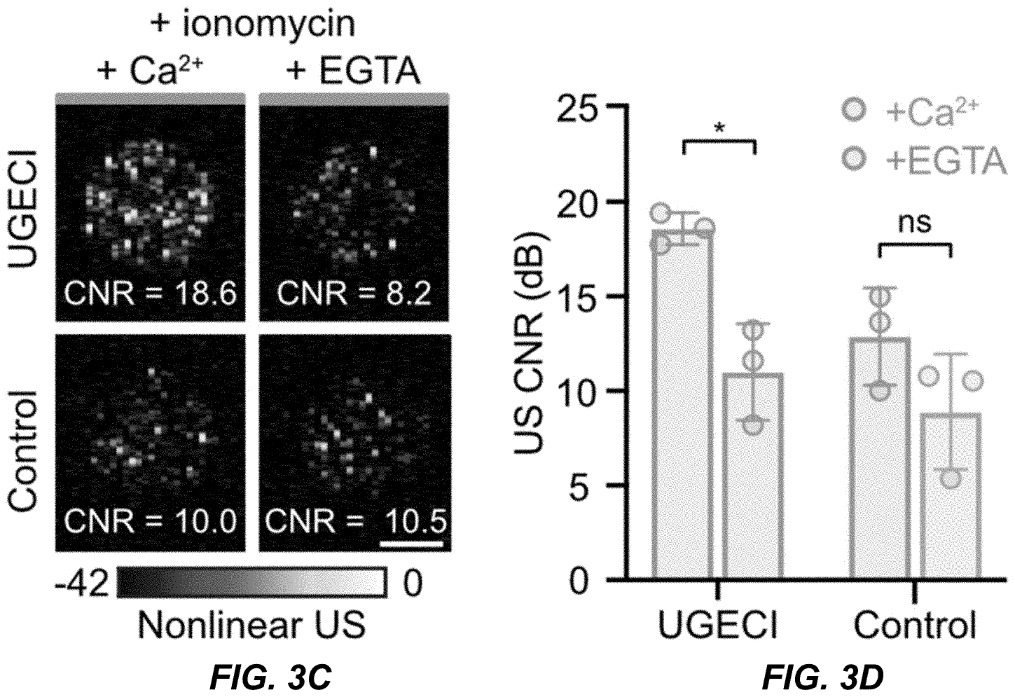

With this design, the UGECI constructs were optimized for mammalian cells. For a higher sensitivity for intracellular sensing, a UGECI construct was constructed with a shorter linker connecting its GvpC and CaM. To express UGECIs in mammalian cells, HEK293T cells were transfected using PEI (N/P ratio=20:1) with a 3-plasmid mixture: one for Anabaena flos-aquae GvpA under a pCMV promoter, one for the UGECI GvpC under a pCMV promoter and one for the rest genes in Anabaena flos-aquae clusters that encode GV (GvpN, GvpJ, GvpK, GvpF, GvpG, GvpW and GvpV). The DNA sequences for the plasmids used in the transfection are as follows: DNA sequence for the pCMV-UGECI-GvpC plasmid (SEQ ID NO: 39); DNA sequence for the pCMV-UGECI-GvpC-Control plasmid (SEQ ID NO: 40); DNA sequence for the pCMV-Ana-GvpA plasmid (SEQ ID NO: 41); and DNA sequence for pCMV-Ana-GvpNV plasmid (SEQ ID NO: 42). It was found that the UGECIs expressed in mammalian cells can produce a calcium-dependent ultrasound contrast, both in permeabilized cells and in living cells with chemically induced calcium dynamics. The dynamic range of UGECIs in mammalian cells was about 10-12 dB (FIGS. 3A-3D), similar to the purified format. The methods and compositions provided herein can be applied in other mammalian cell types and in monitoring other calcium dynamics induced by various stimuli in vivo.

Results

Design of UGECI and In Vitro Characterization

A library of UGECI GvpC was designed with different calcium binding motifs in different positions, was expressed and purified the recombinant GvpC in E. coli, was assembled them onto GVs and their calcium-dependent acoustic properties was characterized through ultrasound. One UGECI embodiment showed around 10 dB enhancement in nonlinear ultrasound contrast in response to 100 μm Ca²⁺ in 37° C., and this enhanced signal was reduced by 9 dB when the Ca²⁺ is chelated by the addition of EGTA, almost fully reversed to the no calcium condition (FIG. 2E). A titration was conducted by mixing UGECIs with varying concentration of Ca²⁺ in 37° C. and ultrasound imaging revealed a K_{d} at 298 nM (FIG. 2F), which is relevant in many physiological conditions. The UGECIs were further characterized by measuring the kinetics in a lab-built stopped-flow ultrasound apparatus and they demonstrated a half-rise time at 28 s when UGECIs were mixed into 14 μm Ca²⁺ in 37° C. For the reverse reaction, UGECIs had a half-decay time at 39s when reversed by 10 mM EGTA from 100 μm Ca²⁺ in 37° C. (FIG. 2G). These results showed that UGECIs can reversibly sense calcium dynamics at sub-micromolar concentrations with a sub-minute time scale.

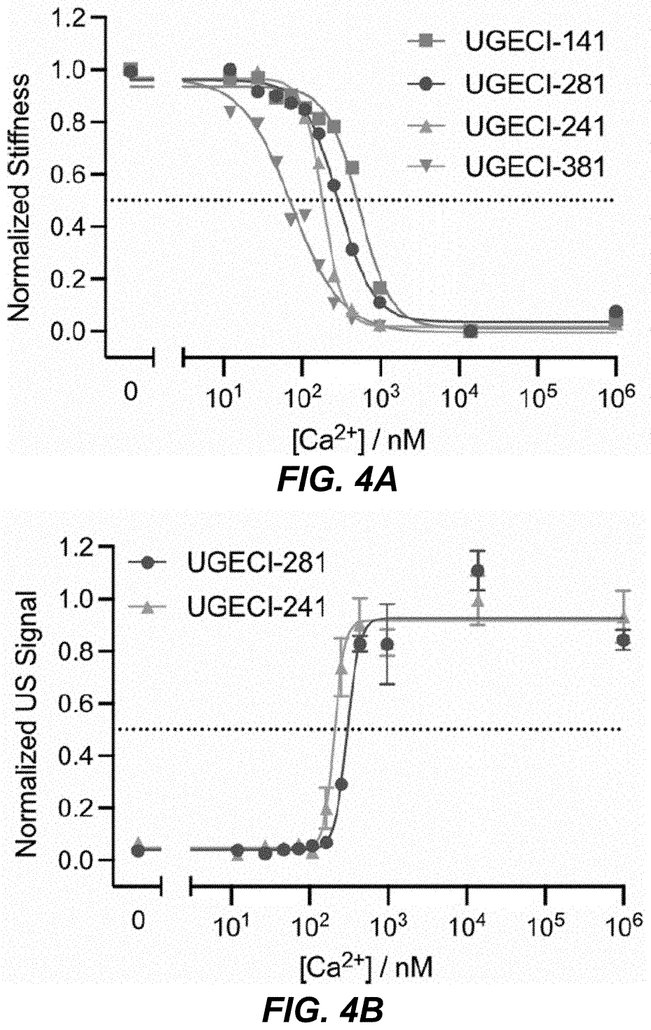

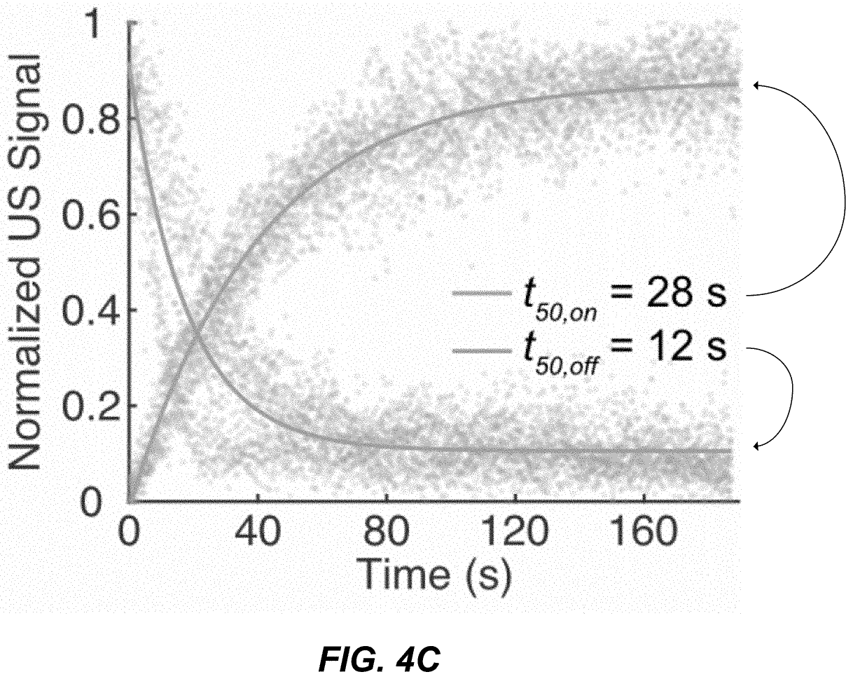

Through the screening of genetic constructs generated by the strategies described in the provisional (FIG. 211), 3 more variants of UGECI were identified (UGECI-141, UGECI-241 and UGECI-381), each with different characteristics (e.g. calcium sensitivity and kinetics). They were characterized along with the original UGECI (named UGECI-281) and it was found that they have the Kd ranging from 73 nM to 529 nM based on the stiffness measurement (FIG. 4A). The UGECI-281 and UGECI-241 were characterized through ultrasound and showed similar Kd, being 298 nM and 205 nM respectively (FIG. 4B). The improved variant UGECI-241 also showed a faster kinetics with a 28-s half-rise time and a 12-s half-decay time (FIG. 4C).

Imaging of Intracellular Calcium Dynamics with UGECIs

The UGECI constructs for mammalian expression were optimized and HEK293T cells were transfected transiently. First, the UGECI-expressing cells were permeabilized with 0.05% saponin and the intracellular environment was equilibrated to 2 mM Ca²⁺ or 5 mM EGTA. A ˜12 dB higher nonlinear ultrasound signal was observed from the cells permeabilized to Ca²⁺ than those permeabilized to EGTA in 37° C. (FIGS. 3A-3B). A control construct was also designed which has the calcium binding motif mutated out and it was tested that in the same condition. It was found that there was no enhancement in response to calcium with the control constructs, indicating that the UGECIs expressed in HEK cells specifically sensed the calcium concentration changes (FIGS. 3A-3B). Second, those constructs were tested with chemically-induced calcium influx in HEK cells. Cells expressing UGECIs and the control were incubated with 10 μm ionomycin and calcium or EGTA, and then imaged with ultrasound. The cells expressing UGECIs had a ˜10 dB higher ultrasound signal when induced Ca²⁺ influx than the EGTA case, while the cells expressing the control construct did not show any significant difference in ultrasound contrast (FIGS. 3C-3D).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A nucleic acid composition, comprising:
one or more promoters operably connected to one or more gas vesicle (GV) polynucleotides comprising:
one or more gas vesicle assembly (GVA) gene(s) encoding one or more GVA protein(s),
one or more gas vesicle structural (GVS) gene(s) encoding one or more GVS protein(s) selected from GvpA and GvpB, and/or
a Ca²⁺-sensing GvpC gene encoding a Ca²⁺-sensing GvpC protein,
wherein the one or more GVA protein(s), the one or more GVS protein(s), and the Ca²⁺-sensing GvpC protein are capable of forming Ca²⁺-sensing gas vesicles (GVs) upon expression in a cell or a cell-like environment.

2. The nucleic acid composition of claim 1, wherein the Ca²⁺-sensing GV comprises a gas enclosed by a protein shell comprising the Ca²⁺-sensing GvpC protein and a GVS protein selected from GvpA and GvpB.

3. The nucleic acid composition of claim 1,
wherein the Ca²⁺-sensing GvpC protein comprises a Ca²⁺-binding domain,
wherein the Ca²⁺-binding domain is capable of binding 1, 2, 3, 4, 5, or 6 Ca²⁺ ions,
wherein the Ca²⁺-sensing GvpC protein is capable of undergoing a first allosteric conformational change upon the Ca²⁺-binding domain binding Ca²⁺ that causes the Ca²⁺-sensing GV to change from a GV stiff state to a GV soft state,
wherein the Ca²⁺-binding domain binding Ca²⁺ comprises the Ca²⁺-binding domain binding 1, 2, 3, 4, 5, or 6 Ca²⁺ ions,
wherein the Ca²⁺-sensing GvpC protein is capable of undergoing a second allosteric conformational change upon the Ca²⁺-binding domain releasing bound Ca²⁺ that causes the Ca²⁺-sensing GV to change from a GV soft state to a GV stiff state, and
wherein the Ca²⁺-binding domain releasing bound Ca²⁺ comprises the Ca²⁺-binding domain releasing 1, 2, 3, 4, 5, or 6 bound Ca²⁺ ions, and
wherein one or more of the mechanical, acoustic, surface, and magnetic properties of the Ca²⁺-sensing GV differ between the GV soft state to the GV stiff state.

4. The nucleic acid composition of claim 1, wherein the acoustic contrast of the Ca²⁺-sensing GV is capable of reversibly changing in response to local Ca²⁺ concentrations.

5. The nucleic acid composition of claim 1, wherein a Ca²⁺-sensing GV is capable of:
(i) binding 1, 2, 3, 4, 5, or 6 Ca²⁺ ions; and
(ii) releasing 1, 2, 3, 4, 5, or 6 bound Ca²⁺ ions,
recurrently in response to changes in Ca²⁺ dynamics.

6. The nucleic acid composition of claim 3, wherein the Ca²⁺-sensing GvpC protein comprises an interaction domain configured to:
bind the Ca²⁺-binding domain upon the Ca²⁺-binding domain binding Ca²⁺,
wherein the first allosteric conformational change comprises the interaction domain binding the Ca²⁺-binding domain; and
detach from the Ca²⁺-binding domain upon the Ca²⁺-binding domain releasing bound Ca²⁺;
wherein the second allosteric conformational change comprises the interaction domain detaching from the Ca²⁺-binding domain.

7. The nucleic acid composition of claim 1, wherein the Ca²⁺-sensing GvpC protein is derived from Anabaena flos-aquae (SEQ ID NO: 33), Halobacterium salinarum (SEQ ID NO: 34), Halobacterium mediterranei (SEQ ID NO: 35), Microchaete diplosiphon (SEQ ID NO: 36), Nostoc sp. (SEQ ID NO: 37), or a combination thereof, and wherein the Ca²⁺-sensing GvpC protein comprises one or more truncation(s), insertion(s), and mutation(s) as compared to the parental GvpC protein from which it is derived.

8. The nucleic acid composition of claim 1, wherein the Ca²⁺-sensing GvpC protein comprises an amino acid sequence that is at least 80%, 85%, 90%, 95%, 98%, 99%, or 100% identical to any one of SEQ ID NOS: 38 and 43-45, or a portion thereof.

9. The nucleic acid composition of claim 6, wherein the interaction domain comprises an amino acid sequence that is at least 80%, 85%, 90%, 95%, 98%, 99%, or 100% identical to any one of SEQ ID NOs: 46-51, or a portion thereof.

10. The nucleic acid composition of claim 3, wherein the Ca²⁺-binding domain comprises an amino acid sequence that is at least 80%, 85%, 90%, 95%, 98%, 99%, or 100% identical to any one of SEQ ID NOs: 52-58, or a portion thereof.

11. The nucleic acid composition of claim 3, wherein the Ca²⁺-binding domain comprises calmodulin (CaM) or a derivative thereof.

12. The nucleic acid composition of claim 6, wherein the interaction domain comprises or is derived from CaMKI.

13. The nucleic acid composition of claim 4, wherein the first allosteric conformational change causes an at least about 1.1-fold reduction in the mechanical stiffness of Ca²⁺-sensing GV.

14. The nucleic acid composition of claim 3, wherein a Ca²⁺-sensing GV in a GV soft state is capable of exhibiting an about 5 dB to about 50 dB enhancement in nonlinear ultrasound contrast as compared to a Ca²⁺-sensing GV in a GV stiff state.

15. The nucleic acid composition of claim 3, wherein a Ca²⁺-sensing GV in a GV soft state is capable of exhibiting an at least about 1.1-fold increase in contrast to noise ratio (CNR) as compared to a Ca²⁺-sensing GV in a GV stiff state.

16. The nucleic acid composition of claim 1, wherein one or more GV polynucleotides comprises:
two or more GVS genes derived from different prokaryotic species;
GVA genes and/or GVS genes from Bacillus Megaterium, Anabaena flos-aquae, Serratia sp., Burkholderia thailandensis, B. megaterium, Frankia sp, Haloferax mediaterranei, Halobacterium sp, Microchaete diplosiphon, Nostoc sp, Halorubrum vacuolatum, Microcystis aeruginosa, Methanosarcina barkeri, Streptomyces coelicolor, and/or Psychromonas ingrahamii;
gvpB, gvpN gvpF, gvpG, gvpL gvpS, gvpK, gvpJ, and/or gvpU from B. megaterium;
gvpA, gvpN, gvpJ, gvpK, gvpF, gvpG, gvpV, and/or gvpW from Anabaena flos-aquae;
gvpR, gvpN, gvpF, gvpG, gvpL, gvpS, gvpK, gvpJ, gvpT and/or gvpU from B. megaterium and gvpA from Anabaena flos-aquae;
gvpA from Anabaena flos-aquae, and gvpN, gvpF, gvpG, gvpL, gvpS, gvpK, gvpJ, and/or gvpU from B. megaterium; and/or
gvpA and/or gvpN from Anabaena flos-aquae, and gvpF, gvpG, gvpL, gvpS, gvpK, gvpJ, and/or gvpU from B. megaterium.

17. The nucleic acid composition of claim 1, wherein the nucleic acid composition is, comprises, or further comprises, one or more vectors,
wherein at least one of the one or more vectors is a viral vector, a plasmid, a transposable element, a naked DNA vector, a lipid nanoparticle (LNP), or any combination thereof,
wherein the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof, and
wherein the transposable element is piggybac transposon or sleeping beauty transposon.

18. An extracellular signal-sensing cell, comprising:
a sensing receptor capable of binding an extracellular signal, wherein sensor receptor signaling triggered by said binding is capable of modulating intracellular Ca²⁺ levels; and
the Ca²⁺-sensing gas vesicles (GVs) encoded by the nucleic acid composition of claim 1.

19. A method of monitoring extracellular signal dynamics, comprising:
administering to a subject an effective amount of the extracellular signal-sensing cells of claim 18; and
applying ultrasound (US) to a target site of the subject, thereby monitoring extracellular signal dynamics.

20. A method of imaging intracellular Ca²⁺ dynamics, comprising:
introducing the nucleic acid composition of claim 1 into the target cells; and
applying ultrasound (US) to said target cells, thereby imaging intracellular Ca²⁺ dynamics.


SEQUENCE LISTING (58 sequences)

SEQ ID NO: 1 - protein, 7 aa, synthetic construct
GSGSGSG

SEQ ID NO: 2 - protein, 5 aa, synthetic construct
GGGGS

SEQ ID NO: 3 - protein, 5 aa, synthetic construct
  REGION 1..5: note = synthetic polypeptide
GSGSG

SEQ ID NO: 4 - protein, 4 aa, synthetic construct
  REGION 1..4: note = synthetic polypeptide
GGGG

SEQ ID NO: 5-7 - skipped (3 entries)

SEQ ID NO: 8 - protein, 4 aa, synthetic construct
  REGION 1..4: note = synthetic polypeptide
GSGS

SEQ ID NO: 9 - protein, 4 aa, synthetic construct
  REGION 1..4: note = synthetic polypeptide
GGGS

SEQ ID NO: 10-11 - skipped (2 entries)

SEQ ID NO: 12 - protein, 6 aa, synthetic construct
  REGION 1..6: note = synthetic polypeptide
GGSGGS

SEQ ID NO: 13 - skipped

SEQ ID NO: 14 - protein, 6 aa, synthetic construct
  REGION 1..6: note = synthetic polypeptide
GGGGGG

SEQ ID NO: 15 - protein, 9 aa, synthetic construct
  REGION 1..9: note = synthetic polypeptide
GGGGGGGGG

SEQ ID NO: 16 - protein, 12 aa, synthetic construct
  REGION 1..12: note = synthetic polypeptide
GGGGGGGGGGGG

SEQ ID NO: 17 - protein, 15 aa, synthetic construct
  REGION 1..15: note = synthetic polypeptide
GGGGGGGGGGGGGGG

SEQ ID NO: 18 - skipped

SEQ ID NO: 19 - protein, 6 aa, synthetic construct
  REGION 1..6: note = synthetic polypeptide
GGSGGS

SEQ ID NO: 20 - protein, 9 aa, synthetic construct
  REGION 1..9: note = synthetic polypeptide
GGSGGSGGS

SEQ ID NO: 21 - protein, 12 aa, synthetic construct
  REGION 1..12: note = synthetic polypeptide
GGSGGSGGSGGS

SEQ ID NO: 22 - protein, 15 aa, synthetic construct
  REGION 1..15: note = synthetic polypeptide
GGSGGSGGSGGSGGS

SEQ ID NO: 23 - skipped

SEQ ID NO: 24 - protein, 6 aa, synthetic construct
  REGION 1..6: note = synthetic polypeptide
GSGGSG

SEQ ID NO: 25 - protein, 9 aa, synthetic construct
  REGION 1..9: note = synthetic polypeptide
GSGGSGGSG

SEQ ID NO: 26 - protein, 12 aa, synthetic construct
  REGION 1..12: note = synthetic polypeptide
GSGGSGGSGGSG

SEQ ID NO: 27 - protein, 15 aa, synthetic construct
  REGION 1..15: note = synthetic polypeptide
GSGGSGGSGGSGGSG

SEQ ID NO: 28 - protein, 5 aa, synthetic construct
  REGION 1..5: note = The entire sequence of amino acids 1-5 can be repeated one or more times
GGGGS

SEQ ID NO: 29 - protein, 10 aa, synthetic construct
  REGION 1..10: note = synthetic polypeptide
GGGGSGGGGS

SEQ ID NO: 30 - protein, 15 aa, synthetic construct
  REGION 1..15: note = synthetic polypeptide
GGGGSGGGGSGGGGS

SEQ ID NO: 31 - protein, 20 aa, synthetic construct
  REGION 1..20: note = synthetic polypeptide
GGGGSGGGGSGGGGSGGGGS

SEQ ID NO: 32 - protein, 25 aa, synthetic construct
  REGION 1..25: note = synthetic polypeptide
GGGGSGGGGSGGGGSGGGGSGGGGS

SEQ ID NO: 33 - protein, 193 aa, Anabaena flos-aquae
MISLMAKIRQEHQSIAEKVAELSLETREFLSVTTAKRQEQAEKQAQELQAFYKDLQETSQQFLSETAQARIAQAEKQAQELLAFHKELQETSQQFLSATAQARIAQAEKQAQELLAFYQEVRETSQQFLSATAQARIAQAEKQAQELLAFHKELQETSQQFLSATADARTAQAKEQKESLLKFRQDLFVSIFG

SEQ ID NO: 34 - protein, 382 aa, Halobacterium salinarum
MSVTDKRDEMSTARDKFAESQQEFESYADEFAADITAKQDDVSDLVDAITDFQAEMTNTTDAFHTYGDEFAAEVDHLRADIDAQRDVIREMQDAFEAYADIFATDIADKQDIGNLLAAIEALRTEMNSTHGAFEAYADDFAADVAALRDISDLVAAIDDFQEEFIAVQDAFDNYAGDFDAEIDQLHAAIADQHDSFDATADAFAEYRDEFYRIEVEALLEAINDFQQDIGDFRAEFETTEDAFVAFARDFYGHEITAEEGAAEAEAEPVEADADVEAEAEVSPDEAGGESAGTEEEETEPAEVETAAPEVEGSPADTADEAEDTEAEEETEEEAPEDMVQCRVCGEYYQAITEPHLQTHDMTIQEYRDEYGEDVPLRPDDKT

SEQ ID NO: 35 - protein, 381 aa, Halobacterium mediterranei
MSVKDKREKMTATREEFAEVQQAFAAYADEFAADVDDKRDVSELVDGIDTLRTEMNSTNDAFRAYSEEFAADVEHFHTSVADRRDAFDAYADIFATDVAEMQDVSDLLAAIDDLRAEMDETHEAFDAYADAFVTDVATLRDVSDLLTAISELQSEFVSVQGEFNGYASEFGADIDQFHAVVAEKRDGHKDVADAFLQYREEFHGVEVQSLLDNIAAFQREMGDYRKAFETTEEAFASFARDFYGQGAAPMATPLNNAAETAVTGTETEVDIPPIEDSVEPDGEDEDSKADDVEAEAEVETVEMEFGAEMDTEADEDVQSESVREDDQFLDDETPEDMVQCLVCGEYYQAITEPHLQTHDMTIKKYREEYGEDVPLRPDDKA

SEQ ID NO: 36 - protein, 162 aa, Microchaete diplosiphon
MTPLMIRIRQEHRGIAEEVTQLFKDTQEFLSVTTAQRQAQAKEQAENLHQFHKDLEKDTEEFLTDTAKERMAKAKQQAEDLFQFHKEMAENTQEFLSETAKERMAQAQEQARQLREFHQNLEQTTNEFLADTAKERMAQAQEQKQQLHQFRQDLFASIFGTF

SEQ ID NO: 37 - protein, 129 aa, Nostoc sp.
MTALMVRIRQEHRSIAEEVTQLFRETHEFLSATTAHRQEQAKQQAQQLHQFHQNLEQTTHEFLTETTTQRVAQAEAQANFLHKFHQNLEQTTQEFLAETAKNRTEQAKAQSQYLQQFRKDLFASIFGTF

SEQ ID NO: 38 - protein, 323 aa, synthetic construct
MISLMAKIRQEHQSIAEKVAELSLETREFLSVTTAKRQEQAEKQAQELQAFYKDLQETSKSKWKQAFNATAVVRHMRKQELLAFHKELQETSQQFLSATAQARIAQAEKQAQELLAFRQDLFVSIFGGGGGSGGGGSGGGGSGGGGSGGGGSGGGGSGGGGSGGGGSDQLTEEQIAEFKEEFSLFAKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAKSLEHHHHHH

SEQ ID NO: 39 - DNA, 5249 bp, synthetic construct
  source 1..5249: mol_type = other DNA
attgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtgatacgaggccctttcactcattaggcaccccaggctttacactttatagcttccggctcgtataatgtgtggaattgtgagcggataacaatttcacacaggaaacagcatcgtgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtacccttcaccatgatcagcctgatggccaagatccggcaggaacaccagtctatcgccgagaaggtggccgagctgagcctggaaacccgcgagtttctgagcgtgaccaccgccaagagacaggaacaggccgagaagcaggcccaggaactccaggccttctacaaggacctccaggaaaccagcaaatccaagtggaagcaagcgtttaatgccacagcagtcgtgcgacacatgagaaaacaggaactgctggcattccacaaagagcttcaggaaacctctcagcagtttctgtccgccaccgcccaggctaggatcgctcaggctgagaaacaggcccaggaactgctggcctttaggcaggacctgttcgtgtccatcttcggcggtggaggtggtagtggtggaggaggcagtggcggtgggggcagcggtggaggtgggagcgaccaactgactgaagagcagatcgcagaatttaaagaggaattctccctatttgccaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtgataatctagaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatcctgcattaatgaatcggccaacgcgcggggagaggcggtttgcgtattggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgccaggtcttggactggtgagaacggcttgctcggcagcttcgatgtgtgctggagggagaataaaggtctaagatgtgcgatagagggaagtcgcattgaattatgtgctgtgtagggatcgctggtatcaaatatgtgtgcccacccctggcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgccataacttcgtataatgtatgctatacgaagttatggcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgcgtttttcaatatt

SEQ ID NO: 40 - DNA, 5249 bp, synthetic construct
  source 1..5249: mol_type = other DNA
attgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtgatacgaggccctttcactcattaggcaccccaggctttacactttatagcttccggctcgtataatgtgtggaattgtgagcggataacaatttcacacaggaaacagcatcgtgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtacccttcaccatgatcagcctgatggccaagatccggcaggaacaccagtctatcgccgagaaggtggccgagctgagcctggaaacccgcgagtttctgagcgtgaccaccgccaagagacaggaacaggccgagaagcaggcccaggaactccaggccttctacaaggacctccaggaaaccagcaaatccaagtggaagcaagcgtttaatgccacagcagtcgtgcgacacatgagaaaacaggaactgctggcattccacaaagagcttcaggaaacctctcagcagtttctgtccgccaccgcccaggctaggatcgctcaggctgagaaacaggcccaggaactgctggcctttaggcaggacctgttcgtgtccatcttcggcggtggaggtggtagtggtggaggaggcagtggcggtgggggcagcggtggaggtgggagcgaccaactgactgaagagcagatcgcagaatttaaagaggaattctccctatttgccaaggacggggatgggacaataacaaccaaggagctggggacggtgatgcggtctctggggcagaaccccacagaagcagagctgcaggacatgatcaatgaagtagctgccgacggtgacggcacaatcgacttccctgagttcctgacaatgatggcaagaaaaatgaaatacagggacacggaagaagaaattagagaagcgttcggtgtgtttgctaaggatggcaatggctacatcagtgcagcagagcttcgccacgtgatgacaaaccttggagagaagttaacagatgaagaggttgatgaaatgatcagggaagcagccatcgatggggatggtcaggtaaactacgaagagtttgtacaaatgatgacagcgaagtgataatctagaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatcctgcattaatgaatcggccaacgcgcggggagaggcggtttgcgtattggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgccaggtcttggactggtgagaacggcttgctcggcagcttcgatgtgtgctggagggagaataaaggtctaagatgtgcgatagagggaagtcgcattgaattatgtgctgtgtagggatcgctggtatcaaatatgtgtgcccacccctggcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgccataacttcgtataatgtatgctatacgaagttatggcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgcgtttttcaatatt

SEQ ID NO: 41 - DNA, 5548 bp, synthetic construct
  source 1..5548: mol_type = other DNA
tccggctcgtataatgtgtggaattgtgagcggataacaatttcacacaggaaacagcatcgtgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtacccttcaccatggccgtggaaaagaccaacagcagcagctccctggccgaagtgatcgacagaatcctggacaagggcatcgtgatcgacgcctgggtgcgcgtgtccctcgtgggaattgagctgctggccatcgaggcccggatcgtgattgccagcgtggaaacctacctgaagtacgccgaggccgtgggcctgacacagagtgctgctgtgcctgcttgataatctagaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttcgtggctgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagagacaattaacttcgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaagttctcagatcctgcattaatgaatcggccaacgcgcggggagaggcggtttgcgtattggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgccaggtcttggactggtgagaacggcttgctcggcagcttcgatgtgtgctggagggagaataaaggtctaagatgtgcgatagagggaagtcgcattgaattatgtgctgtgtagggatcgctggtatcaaatatgtgtgcccacccctggcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgccataacttcgtataatgtatgctatacgaagttatggcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgcgtttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtgatacgaggccctttcactcattaggcaccccaggctttacactttatagct

SEQ ID NO: 42 - DNA, 10484 bp, synthetic construct
  source 1..10484: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttattctttcctgcgttatcccctgattctgtggataaccgtattaccgcctttgagtgagctgataccgctcgccgcagccgaacgaccgagcgcagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgcgagggtctccgccaccatggtgaccaccaccaaagtgaaccacaagcgggccgtgctgagactgaggcctggccagtttgtcgtgacccccgccattgagcgggtggccattagagccctgagatacctgaagtccggcttccccgtgcacctgagaggacctgccggaaccggcaagaccacactggccatgcacctggccaactgcctggacagacccgtgatgctgctgttcggcgacgaccagttcaagagcagcgacctgatcggcagcgagagcggctacacccacaagaaggtgctggacaactacatccacagcgtcgtgaagctggaagatgagttcaagcagaactgggtggacagcagactgaccctggcctgccgggaaggcttcaccctggtgtacgacgagttcaaccggtccagacccgaagtgaacaacgtgctgctgagcgccctggaagagaagatcctgagcctgccccccagcagcaaccagccagagtacctgagcgtgaacccccagttcagagtgatcttcaccagcaaccccgaggaatatgccggcgtgcacagcacccaggacgccctgatggaccggctcgtgaccatctccatgcccgagcccgatgagatcacccagaccgagatcctgatccagaaaacaaacatcgaccgcgagagcgccaacttcatcgtgcggctcgtgaagtccttcagactggccacaggcgccgagaaaaccagcggcctgagaagctgcctgatgatcgccaaagtgtgcgccgacaacaacatccccgtgaccaccgagagcctggacttccccgatatcgccatcgacatcctgttcaaccgcagccacctgagcatgagcgagtccaccaacatctttctggaactgctggataagttcagcgccgaggaactggaaatcctgaacaacagagtgaccggcgacaacgacttcctgatcgacaacagccagttcgtgtcccagcagctggccggacagcccaacggagcgccaggttccggggctactaacttcagcctccttaaacaggctggcgatgtggaagaaaatcccggaccagtgctgcccaccagaccccagaccaacagcagccggaccatcaacaccagcacccagggcagcaccctggccgacatcctggaaagagtgctggacaagggcatcgtgatcgccggggacatcagcatctctatcgccagcaccgagctggtgcacatccggatcagactgctgatctccagcgtggacaaggccaaagagatgggcatcaactggtgggagagcgacccctacctgagcaccaaggcccagagactggtggaagagaaccagcagctccagcacagactggaaagcctggaagccaagctgaacagcctgaccagcagcagcgtgaaagaggaaatccccctggccgccgacgtgaaggacgacctgtatcagaccagcgccaagatccccagccccgtggatacccctatcgaggtgctggacttccaggcccagtctagcggcggcacacccccttacgtgaacacctctatggaaatactggattttcaggctcagacctccgccgagagcagcagccctgtgggctctaccgtggaaatactggacttccaggcacagacaagcgaggaatccagctcccccgtggtgtccacagtggaaatactggatttccaggcccagacttctgaagagtccagctctccagtgggaagcactgtggaaatactggactttcaggcccagaccagtgaagagatccccagctctgtggaccccgccatcgatgtgggtgccccgggatctggcgcaacaaattttagtctcctcaagcaggcaggagatgtcgaggaaaaccctggacccgtggtgtgcacccctgccgagaacttcaacaacagcctgacaatcgccagcaagcccaagaacgaggccggactggctcctctgctgctgaccgtgctggaactcgtgcggcagctgatggaagcccaagtgatccggcggatggaagaggacctgctgagcgagcccgacctggaaagagccgccgatagcctccagaagctggaagaacagatcctgcacctgtgcgagatgttcgaggtggaccccgccgacctgaacatcaacctgggcgagatcggcaccctgctgcctagcagcggcagctactatcctggccagccaagcagcagaccctctgtgctggaactgctggaccggctgctgaacaccggcatcgtggtggacggcgagattgacctgggaatcgcccagatcgacctgatccacgccaagctgagactggtgctgacctccaagcccatcggggcacctggctcaggagcgaccaacttctcattgctcaaacaagccggagatgttgaggagaatccaggccctgtgagcatccccctgtacctgtacggcatcttccccaacaccatccccgagacactggaactggaaggcctggacaagcagcccgtgcacagccaggtggtggacgagttctgcttcctgtacagcgaggcccggcaggaaaagtacctggccagcagacggaacctgctgacccacgagaaggtgctggaacagaccatgcacgccggcttcagagtgctgctgcccctgagatttggcctggtcgtgaaggactgggagacaatcatgagccagctgatcaacccccacaaggaccagctgaaccagctgttccagaagctggccggcaagcgggaagtgtccatcaagattttctgggacgccaaggccgagcttcagaccatgatggaaagccaccaggacctgaagcagcagcgggacaacatggaaggcaagaaactgagcatggaagaagtgatccagatcgggcagctgatcgagatcaacctgctggcccggaagcaggccgtgatcgaggtgttcagccaggaactgaaccccttcgcccaggaaatcgtggtgtccgaccccatgaccgaggaaatgatctacaacgccgccttcctgatcccctgggagagcgagagcgagttcagcgagcgggtggaagtgatcgaccagaagttcggcgaccggctgcggatcagatacaacaacttcaccgccccctacacctttgcccagctggactctggcgctcccgggtcaggtgccactaatttttcattgttgaagcaagctggggatgttgaagagaacccagggcctgtgctgaccaaactcctgctgctgcccatcatgggacccctgaatggcgtcgtgtggatcgccgagcagatccaggaacggaccaacaccgagttcgacgcccaggaaaacctgcacaaacagctgctgagcctccagctgagcttcgacatcggcgagatcggagaggaagagttcgagattcaggaagaggaaatcctgctgaagatccaggccctggaagaagaggcccggctggaactggaagccgagcaggaagaagccagactggaactggaagctgaacaggaagatttcgagtacccaccacagttcaccgccgaagtgaacaaggatcagcatctggtgctgctgcccggtgccccgggatctggcgctactaacttcagcctccttaaacaggctggcgatgtggaagaaaatcccggaccagtggaactggaaaacctgtacacctacgcctttctggaaatccccagcagccccctgatcctgcctcagggcgctgctaatcaggtggtgctgatcaacggcaccgagctggccgccatcgtggaacctggcatcttcctggaatccttccagaacaacgacgagaagatcatccagatggccctgagccacgacagagtgatctgcgagctgttccagcagatcaccgtgctgcccctgagattcggcacctacttcaccagcaccaacaacctgctgaaccacctgaagtcccacgagaaagagtaccagaacaagctggaaaagatcaacgggaagaacgagttcaccctgaagctgatcccccggatgatcgaggaaatcgtgccctctgagggcggaggcaaggactacttcctggccaagaagcagcgctaccagaatcagaacaacttctctatcgcccaggccgccgagaagcagaacctgatcgacctgatcaccaaagtgaaccagctgcccgtggtggtgcaggaacaggaagaacagatccaaatctacctgctggtgtcctgccaggataagaccctgctgctggaacagttcctgacctggcagaaagcctgccccaagtgggatctgctgctgggcgactgcctgcccccctaccactttatcggagcgccaggttccggggcaacaaattttagtctcctcaagcaggcaggagatgtcgaggaaaaccctggacccgtgatcaagaacatccaggtgttctttatgaaaaccatcagcaaccggtccatcagccgggccaagatcagcaccatgcccagacctaagagcgacgccagcagccagctggacctgtacaagatggtcaccgagaagcagagaatccagcgggatatgtacagcatcaaagaacggatgggcctgctccagcagcggctggacatcctgaaccagcagatcgaggccaccgagaaaaccatccacaagctgcggcagccccacagcaacaccgcccagaatatcgtgcggagcaacatcttcgtggaaagcaacaactaccagaccttcgaggtggaatacggggcacctggctcaggagccactaatttttcattgttgaagcaagctggggatgttgaagagaacccagggcctgtggtgtccaagggcgaggaactgttcaccggcgtggtgcccatcctggtggaactggatggcgacgtgaacggccacaagttcagcgtgtccggcgagggcgaaggcgacgccacatacggaaagctgaccctgaagttcatctgcaccaccggcaagctgcccgtgccttggcctaccctcgtgaccacactgacctacggcgtgcagtgcttcgccagataccccgaccacatgaagcagcacgatttcttcaagagcgccatgcccgagggctacgtgcaggaacggaccatcttcttcaaggacgacggcaactacaagacaagagccgaagtgaagttcgagggcgacaccctcgtgaaccggatcgagctgaagggcatcgacttcaaagaggatggcaacatcctgggccacaagctggagtacaactacaacagccacaaggtgtacatcaccgccgacaagcagaaaaacggcatcaaagtgaacttcaagacccggcacaacatcgaggacggcagcgtgcagctggccgaccactaccagcagaacacccccatcggagatggccccgtgctgctgcccgacaaccactacctgagcacacaaagcgccctgagcaaggaccccaacgagaagcgggaccacatggtgctgctggaatttgtgaccgccgctggcatcaccctgggcatggacgagctgtacaagtgactcgagtctagagggccccgtggctgtaatctagaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttcgtggctgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagagacaattaacttcgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaagttctcagatcctgcattaatgaatcggccaacgcgcggggagaggcggtttgcgtattggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgccaggtcttggactggtgagaacggcttgctcggcagcttcgatgtgtgctggagggagaataaaggtctaagatgtgcgatagagggaagtcgcattgaattatgtgctgtgtagggatcgctggtatcaaatatgtgtgcccacccctggcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgccataacttcgtataatgtatgctatacgaagttatggcatgaccaaaatcccttaacgtgagttttcgttccactgagcgtcagaccccgtagaaaagatcaaaggatcttc

SEQ ID NO: 43 - protein, 303 aa, synthetic construct
MISLMAKIRQEHQSIAEKVAELSLETREFLSVTTAKRQEQAEKQAQELQAFYKDLQETSRWKKNFIAVSAANRFKKRKQELLAFHKELQETSQQFLSATAQARIAQAEKQAQELLAFRQDLFVSIFGGGGGSGGGGSGGGGSGGGGSDQLTEEQIAEFKEEFSLFDKDGDGTITTKELGTVMRSLGQNPTEAELQDMINEVDADGDGTIDFPEFLTMMARKMKYRDTEEEIREAFGVFAKDGNGYISAAELRHVMTNLGEKLTDEEVDEMIREADIDGDGQVNYEEFVQMMTAKSLEHHHHHH

SEQ ID NO: 44 - protein, 303 aa, synthetic construct
MISLMAKIRQEHQSIAEKVAELSLETREFLSVTTAKRQEQAEKQAQELQAFYKDLQETSKSKWKQAFNATAVVRHMRKQELLAFHKELQETSQQFLSATAQARIAQAEKQAQELLAFRQDLFVSIFGGGGGSGGGGSGGGGSGGGGSDQLTEEQIAEFKEEFSLFAKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAKSLEHHHHHH

SEQ ID NO: 45 - protein, 324 aa, synthetic construct
MISLMAKIRQEHQSIAEKVAELSLETREFLSVTTAKRQEQAEKQAQELQAFYKDLQETSRWKKAFIAVSAANRFKKRKQELLAFHKELQETSQQFLSATAQARIAQAEKQAQELLAFRQDLFVSIFGGGGGSGGGGSGGGGSGGGGSGGGGSGGGGSGGGGSGGGGSDQLTEEQIAEFKEEFSLFAKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAKTSGSHHHHHH

SEQ ID NO: 46 - protein, 19 aa, synthetic construct
RRKLKAAVKAVVASSRLGS

SEQ ID NO: 47 - protein, 26 aa, synthetic construct
KRRWKKNFIAVSAANRFKKISSSGAL

SEQ ID NO: 48 - protein, 370 aa, synthetic construct
MLGAVEGPRWKQAEDIRDIYDFRDVLGTGAFSEVILAEDKRTQKLVAIKCIAKEALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGTSQEGQGQTASHGELLTPVAGGPAAGCCCRDCCVEPGTELSPTLPHQL

SEQ ID NO: 49 - protein, 19 aa, synthetic construct
RWKKNFIAVSAANRFKKRK

SEQ ID NO: 50 - protein, 19 aa, synthetic construct
KSKWKQAFNATAVVRHMRK

SEQ ID NO: 51 - protein, 19 aa, synthetic construct
RWKKAFIAVSAANRFKKRK

SEQ ID NO: 52 - protein, 147 aa, synthetic construct
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 53 - protein, 147 aa, synthetic construct
DQLTEEQIAEFKEAFSLFDKDGDGTITTKELGTVMRSLGQNPTEAELQDMINEVDADGDGTIDFPEFLTMMARKMKYTDSEEEIREAFGVFDKDGNGYISAAELRHVMTNLGEKLTDEEVDEMIREADIDGDGQVNYEEFVQMMTAK

SEQ ID NO: 54 - protein, 147 aa, synthetic construct
DQLTEEQIAEFKEEFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 55 - protein, 147 aa, synthetic construct
DQLTEEQIAEFKEEFSLFAKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 56 - protein, 147 aa, synthetic construct
DQLTEEQIAEFKEEFSLFDKDGDGTITTKELGTVMRSLGQNPTEAELQDMINEVAADGDGTIDFPEFLTMMARKMKYRDTEEEIREAFGVFDKDGNGYISAAELRHVMTNLGEKLTDEEVDEMIREADIDGDGQVNYEEFVQMMTAK

SEQ ID NO: 57 - protein, 147 aa, synthetic construct
DQLTEEQIAEFKEEFSLFDKDGDGTITTKELGTVMRSLGQNPTEAELQDMINEVDADGDGTIDFPEFLTMMARKMKYRDTEEEIREAFGVFAKDGNGYISAAELRHVMTNLGEKLTDEEVDEMIREADIDGDGQVNYEEFVQMMTAK

SEQ ID NO: 58 - protein, 147 aa, synthetic construct
DQLTEEQIAEFKEEFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREAAIDGDGQVNYEEFVQMMTAK